(19) United States
(12) Patent Application Publication
(10) Pub. No.: US 2026/0305959 A1
(43) Pub. Date: Oct. 8, 2026

(54) PERFORMANCE OF AI ALGORITHM FOR DETECTION OF BLOOD PRESSURE

(71) Applicant: Aktiia SA, Neuchâtel (CH)

(72) Inventors: Josep Sola, Cormondrèche (CH); Sibylle Fallet, Corcelles-Près-Concise (CH); Andreu Arderiu, Lausanne (CH); Bastien Orset, Avenue Ignace-Padereweski 26c (CH); Serj Haddad, Route D'arnier 24 (CH); Theodore Gkamaletsos, Chemin De Longeraie 4 (CH); Olivier Grossenbacher, Neuchâtel (CH); Sasan Yazdani, Vufflens-La-Ville (CH); Neil Puri, 2505 Caminito Muirfield, CA (US); Mattia Bertschi, Epalinges (CH)

(21) Appl. No.: 19/229,560

(22) Filed: Jun. 5, 2025

Related U.S. Application Data
(63) Continuation of application No. 19/172,404, filed on Apr. 7, 2025.

(51) Int. Cl. A61B 5/021; A61B 5/00; A61B 5/026

(52) U.S. Cl. CPC A61B 5/021; A61B 5/0261; A61B 5/6802; A61B 5/7221; A61B 5/742

(57) ABSTRACT

A system for determining a blood pressure value of a user includes a processor programmed to perform operations that include receiving a signal from a sensor, the sensor adapted to non-invasively receive the signal from the user, validating that at least one of the sensor or the received signal satisfies one or more predetermined criteria, delivering the signal as an input to a model, the model configured to analyze the signal and, without any further calibration, returns the blood pressure value as an output, and delivering the output blood pressure value to the user.

### CROSS-REFERENCE TO RELATED APPLICATIONS

This present application claims priority to and is a continuation of U.S. application Ser. No. 19/172,404, entitled “Performance of Artificial Intelligence (AI) Algorithm for Detection of Blood Pressure,” and filed on Apr. 7, 2025, which is herein incorporated by reference in its entirety.

### TECHNICAL FIELD

This specification relates to systems and methods for measuring blood pressure and, in particular, to the determination of blood pressure values using non-invasive sensors.

### BACKGROUND

Blood pressure is typically measured using a device called a sphygmomanometer. Blood pressure measurements are recorded in millimeters of mercury (mmHg) and consists of two values: the systolic pressure and the diastolic pressure. Systolic pressure measures the force in the arteries when the heart beats, while diastolic pressure measures it when the heart is at rest. In most cases, an inflatable cuff is placed around the upper arm, inflated to restrict blood flow, and a stethoscope is used to listen as air is released to determine the systolic and diastolic readings. Due to the need for such specialized equipment, most individuals have their blood pressure measured on a relatively infrequent basis (e.g., when visiting a doctor).

Cuff-free blood pressure measurements can be performed using sensors and algorithms to provide non-invasive readings without using an inflatable cuff. Techniques like photoplethysmography (PPG) and pulse transit time (PTT) may be used. PPG involves the use of optical sensors that track blood volume changes by analyzing how light reflects through the skin, correlating it with heart rate and blood flow. Likewise, PTT measures the time it takes a pulse wave to travel between two body points, such as from the heart to the wrist, and uses this data with heart rate and arterial stiffness to estimate blood pressure. While cuff-free systems may be convenient, their precision often depends on calibration measurements (e.g., captured using an inflatable cuff system). Due to such calibration requirements, it can be difficult for individuals to obtain, and maintain, accurate blood pressure measurements from cuff-free systems.

### SUMMARY

At least one aspect of the present disclosure is directed to a system for determining a blood pressure value of a user. The system includes a processor programmed to perform operations that include receiving a signal from a sensor, the sensor adapted to non-invasively receive the signal from the user, validating that at least one of the sensor or the received signal satisfies one or more predetermined criteria, delivering the signal as an input to a model, the model configured to analyze the signal and, without any further calibration, returns the blood pressure value as an output, and delivering the output blood pressure value to the user.

In some embodiments, the sensor includes a video acquisition system. In some embodiments, the video acquisition system is embedded within a smartphone, a tablet, or any handheld electronic device. In some embodiments, the sensor includes an optical sensor. In some embodiments, the optical sensor is housed in a wearable device, a smartwatch, a wristband, or a finger-worn ring.

In some embodiments, the signal includes a pulsatility signal from the arteries of the user. In some embodiments, the signal includes a pulsatility signal received during at least the duration of one heart cycle of the user. In some embodiments, the operations further comprise calculating a signal quality criterion.

In some embodiments, the system is regulated as a medical device according to an applicable regulatory regime. In some embodiments, the system is regulated as software as a medical device (SaMD) or as medical device software (MDSW). In some embodiments, the predetermined criteria comprises performance requirements mandated by an applicable regulatory regime. In some embodiments, the predetermined criteria is different based the regulatory regime appliable to the user. In some embodiments, identification of the regulatory regime applicable to the user is based on a selection performed by the user in a user interface of the system. In some embodiments, identification of the regulatory regime applicable to the user is automatically performed via localization mechanisms of the system.

In some embodiments, validating the sensor includes verifying that the sensor is in a list of compatible sensors for the system in a regulatory territory applicable for the user. In some embodiments, validating the sensor comprises verifying that the sensor generates signals that comply with one or more predetermined criteria. In some embodiments, validating the received signal includes evaluating signal integrity based on one or more of signal-to-noise ratio, waveform stability, artifact detection, or physiological plausibility. In some embodiments, validating the received signal includes evaluating contextual information including at least one of user posture, motion state, sleep condition, or physical activity, obtained from one or more supplementary sensors or user-provided inputs. In some embodiments, validating the received signal includes confirming compliance of the received signal with regulatory standards or guidelines applicable to the territory in which the blood pressure value is delivered. In some embodiments, the processor is programmed to perform operation that include delivering at least one personal characteristic of the user as an input to the model, wherein the model is configured to analyze the signal and the at least one personal characteristic and, without any further calibration, return the blood pressure value based on the signal and the at least one personal characteristic.

In some embodiments, the model is located on a same device as the sensor. In some embodiments, the model is located remote from the sensor. In some embodiments, the processor is located remote from the sensor. In some embodiments, delivering the output blood pressure value comprises delivering the blood pressure value in a user-accessible format. In some embodiments, delivering the output blood pressure value comprises delivering the blood pressure to a device on which the sensor is embedded.

Another aspect of the present disclosure is directed to a system for determining a blood pressure value of a user. The system includes a processor in communication with a calculation model, wherein the calculation model is configured to determine the blood pressure value of the user based on signals collected from the user. The processor is programmed to perform operations that include identifying whether a measurement device is a verified device, verified devices being defined as measurement devices that have completed a qualification process. The qualification process includes analyzing a shape of a signal received from the measurement device, determining configuration settings for the measurement device such that the shape of the signal will have predetermined characteristics, storing the configuration settings, and qualifying the measurement device as a verified device. If the measurement device is a verified device, the processor is configured to perform operations that include configuring the measurement device according to configuration settings determined during the qualification process, receiving a signal from the measurement device, and delivering the signal to the calculation model to determine the blood pressure value of the user.

In some embodiments, the operations performed by the processor are at least one of: (i) performed on a server remote from the measurement device, (ii) performed on the measurement device, or (iii) partially performed on the server remote from the measurement device and partially performed on the measurement device. In some embodiments, the calculation model is hosted on a server remote from the measurement device.

In some embodiments, the signals include optical signals. In some embodiments, the signals include PPG signals. In some embodiments, the measurement device comprises a smartphone. In some embodiments, the measurement device includes a wearable device.

In some embodiments, the qualification process is performed outside of the processor. In some embodiments, the qualification process if performed outside of the system. In some embodiments, the qualification process includes benchtop testing using at least one of simulated data and real subject data.

In some embodiments, analyzing the shape of the signal includes evaluating one or more morphological, statistical, physiological, or model-derived characteristics of the signal, wherein the characteristics include at least one of amplitude metrics, temporal features, waveform slopes, baseline stability, distortion or noise metrics, physiological landmark presence, inter-feature relationships, quality indicators generated by the calculation model, or correlation measures with reference signals. In some embodiments, the configuration settings include at least one of optical signal acquisition parameters, analog front-end processing parameters, digital signal processing parameters, data encoding parameters, or environmental adaptation parameters, including but not limited to light intensity, light exposure time, wavelength selection, illumination mode, emitter-detector spatial configuration, sampling rate, gain control, filtering characteristics, resolution, dynamic range, signal-to-noise ratio optimization, compression settings, frame rate, white balance, autofocus mode, exposure compensation, stabilization settings, ambient light correction, temperature compensation, or data transmission format, wherein such configuration settings are set to optimize signal quality, maintain measurement consistency, or ensure regulatory compliance across different device types and operating conditions.

In some embodiments, configuring the measurement device according to the configuration settings includes at least one of: (i) modifying settings of the measurement device or (ii) determining that the settings of the measurement device already align with the configuration settings. In some embodiments, the operations further include validating a quality of the signal received from the measurement device before delivering the signal to the calculation model. In some embodiments, validating the quality of the signal includes evaluating at least one of signal integrity characteristics, physiological plausibility criteria, contextual conditions of the user, environmental conditions during measurement, or compliance with predetermined regulatory criteria.

In some embodiments, the operations include identifying personal characteristics of the user and delivering the personal characteristics for use by the calculation model along with the signal. In some embodiments, the operations include delivering the blood pressure value to the user. In some embodiments, the delivered blood pressure value is configured to be displayed to the user on a display. In some embodiments, the display is part of the measurement device. In some embodiments, if the measurement device is not included in the list of verified devices, the processor performs operations that include causing the measurement device to display a message indicating that the measurement device is not supported for interaction with the calculation model.

Another aspect of the present disclosure is directed to a system for determining a blood pressure value of a user. The system includes a processor programmed to perform operations that include receiving a signal from a sensor adapted to receive the signal from the user, delivering the signal as an input to a model that returns the blood pressure value as an output. The model is developed according to a process that includes training the model using a first data set of signals, refining the trained model using a second data set of signals wherein the signals of the second data set are annotated with information correlated with blood pressure values collected using a reference methodology on a first cohort of subjects, and validating the refined model to ensure conformance with a validation criteria by comparing blood pressure values returned by the refined model against blood pressure values collected on a second cohort of subjects using the reference methodology.

In some embodiments, the sensor is embedded within a smartphone. In some embodiments, the sensor is embedded within a wearable device. In some embodiments, the signal includes a pulsatility signal from the arteries of the user.

In some embodiments, the processor is programmed to perform operations that include validating a quality of the signal received from the sensor. In some embodiments, validating the quality of the signal includes confirming that the signal meets a predetermined quality threshold under varying real-world conditions. In some embodiments, validating the quality of the signal includes evaluating at least two of signal integrity, physiological conditions and external factors.

In some embodiments, the validating the quality of the signal is performed within the model. In some embodiments, validating the quality of the signal is performed outside the model.

In some embodiments, the first data set includes pulsatility signals collected by first data set sensors, wherein the first data set sensors are at least one of (i) of the same type as the sensor, (ii) of a different type as the sensor, or (iii) comprises of multiple sensor types. In some embodiments, the first data set includes pulsatility signals collected by first data set sensors, wherein the first data set sensors are at least one of: (i) located at the same body location as the sensor, (ii) located at a different body location as the sensor, or (iii) located at multiple body locations. In some embodiments, the first data set includes at least one of: (i) synthetic data generated by a digital or an analog simulator or (ii) data generated by randomly augmenting the first data set. In some embodiments, at least some of the signals in the first data set are annotated with information correlated with blood pressure values collected using at least one of (i) the reference methodology, (ii) a methodology different than the reference methodology, or (iii) multiple methodologies.

In some embodiments, the first data set is collected using scalable data collection methodologies that require at most a minimal level of clinical supervision. In some embodiments, the first cohort of subjects have characteristics similar to those of the second cohort of subjects. In some embodiments, the characteristics include statistical distributions of at least one of systolic blood pressure, diastolic blood pressure, age, biological sex, or personal characteristics. In some embodiments, the personal characteristics are selected from the group consisting of height, weight, body mass index, and skin tone.

In some embodiments, the signals of the second data set are annotated with information correlated with blood pressure values collected using multiple reference methodologies. In some embodiments, the reference methodology includes collecting measurements using at least one of an auscultatory blood pressure monitor, an invasive blood pressure monitor, a blood pressure monitor required for regulatory compliance, or a blood pressure monitor recognized by a regulatory body.

In some embodiments, the validation criteria includes a bias of less than 5 mmHg and a precision within one of 8 mmHg or 10 mmHg. In some embodiments, the model returns the blood pressure value as the output without any further calibration. In some embodiments, the model returns the blood pressure value as the output following normalization using personal characteristic data from the user. In some embodiments, the model returns the blood pressure value as the output following calibration using physiological data from the user. In some embodiments, the model returns the blood pressure value as the output following calibration using blood pressure data from the user. In some embodiments, the operations include presenting the blood pressure value to the user. In some embodiments, the operations include presenting a trend of blood pressure values to the user.

### BRIEF DESCRIPTION OF THE DRAWINGS

The accompanying figures, which are included as part of the present specification, illustrate the presently preferred embodiments and together with the general description given above and the detailed description of the preferred embodiments given below serve to explain and teach the principles described herein.

FIG. 1 is a block diagram of a blood pressure measurement system in accordance with aspects described herein;

FIG. 2A is a front view of a user device in accordance with aspects described herein;

FIG. 2B is a rear view of a user device in accordance with aspects described herein;

FIG. 3A is a front view of a measurement configuration in accordance with aspects described herein;

FIG. 3B is a rear view of a measurement configuration in accordance with aspects described herein;

FIG. 4 is a view of a measurement configuration in accordance with aspects described herein;

FIG. 5 is a view of a measurement configuration in accordance with aspects described herein;

FIG. 6A is a top view of a user device in accordance with aspects described herein;

FIG. 6B is a side view of a user device in accordance with aspects described herein;

FIG. 7A is a top view of a user device in accordance with aspects described herein;

FIG. 7B is a side view of a user device in accordance with aspects described herein;

FIG. 8A is a top view of a user device in accordance with aspects described herein;

FIG. 8B is a side view of a user device in accordance with aspects described herein;

FIG. 9 is a flowchart of a method of operation for a calibration-free BP determination system in accordance with aspects described herein;

FIG. 10 is a flowchart of a method for qualifying a user device for BP determination in accordance with aspects described herein;

FIG. 11 is a flowchart of a method for performing a BP determination using a verified measurement device in accordance with aspects described herein;

FIG. 12 is a flowchart of a method for training a BP estimation model in accordance with aspects described herein; and

FIG. 13 illustrates an example computing device.

While the present disclosure is subject to various modifications and alternative forms, specific embodiments thereof have been shown by way of example in the drawings and will herein be described in detail. The present disclosure should not be understood to be limited to the particular forms disclosed, but on the contrary, the intention is to cover all modifications, equivalents, and alternatives falling within the spirit and scope of the present disclosure.

### DETAILED DESCRIPTION

Traditionally, blood pressure (BP) measurement has been dominated by cuff-based methodologies, notably the auscultatory and oscillometric methods. The auscultatory method involves wrapping an inflatable cuff (e.g., of a sphygmomanometer) around the upper arm of the subject, inflating it to occlude arterial blood flow, and gradually deflating the cuff while using a stethoscope to detect Korotkoff sounds. The initial sound signifies systolic BP, while the disappearance of sound indicates diastolic BP. Although clinically accurate and considered the gold standard, this method requires extensive training and can introduce observer-related errors. The oscillometric method, widely used in automatic cuff-based BP monitoring devices, measures oscillations in cuff pressure caused by arterial pulsations during cuff deflation. These oscillations are algorithmically interpreted to determine systolic and diastolic BP values. Oscillometric devices offer greater ease of use and are suitable for both clinical and home settings, yet their accuracy may be compromised by factors such as arterial stiffness, arrhythmias, and incorrect cuff positioning.

Due to the need for specialized equipment, most individuals have their BP measured on a relatively infrequent basis (e.g., when visiting a doctor). However, cuff-free BP measurements may be performed using sensors and algorithms to provide non-invasive readings without using an inflatable cuff. For example, PPG-based techniques involve the use of optical sensors that track blood volume changes by analyzing how light reflects through the skin, correlating it with heart rate and blood flow. Likewise, PTT-based techniques measure the time it takes a pulse wave to travel between two body points, such as from the heart to the wrist, and uses this data with heart rate and arterial stiffness to estimate BP.

While these cuff-free systems may be convenient, their precision often depends on calibration or initialization measurements (e.g., captured using an inflatable cuff system). Often times, the accuracy of such systems critically depends on individual-specific calibration, requiring frequent recalibration due to physiological variability. In some cases, repeated, periodic calibration (e.g., daily, weekly, monthly, etc.) is needed to maintain measurement reliability. Due to such calibration requirements, it can be difficult for individuals to obtain, and maintain, accurate blood pressure measurements from cuff-free systems. For example, some wearable devices leverage optical sensors to detect arterial pulsatility signals for cuffless BP monitoring. However, such devices still require user-driven calibrations, often repeated monthly using traditional cuff measurements, to maintain clinical reliability. Consequently, such devices, though widely adopted for personal health tracking, cannot achieve true standalone accuracy without periodic calibration. Likewise, Continuous Noninvasive Arterial Pressure (CNAP) monitoring merges aspects of invasive arterial monitoring and noninvasive cuff techniques. CNAP uses finger cuffs applying PPG coupled with vascular unloading to continuously measure BP. Despite continuous measurement capability, CNAP devices require periodic calibration against traditional cuff-based measurements to maintain accuracy, particularly when influenced by changes in peripheral circulation and vasomotor tone. As such, it is desired to achieve calibration-free cuffless BP monitoring, aspiring to provide accurate, non-invasive, and maintenance-free solutions.

Accordingly, improved systems and methods for non-invasive BP estimation are provided herein. In some examples, these systems and methods provide clinically-accurate BP estimation without the need for calibration (or initialization) using an inflatable cuff device. As such, these systems and methods provide non-invasive BP estimation techniques having improved accuracy, usability, scalability, and/or commercial viability.

In some embodiments, a system for determining a BP value of a user includes a processor programmed to receive a signal from a sensor. In some examples, the sensor is adapted to non-invasively receive the signal from the user. The processor is further programmed to validate that at least one of the sensor or the received signal complies with one or more predetermined criteria and deliver the signal as an input to a model. In some examples, the model is configured to analyze the signal and, without any further calibration, return the BP value as an output. The processor may be programmed to deliver the output BP value to the user.

In some embodiments, a system for determining a BP value of a user includes a processor in communication with a calculation model. In some examples, the calculation model is configured to determine the BP value of the user based on signals collected from the user. The processor is programmed to identify whether a measurement device is a verified device. In some examples, a verified device is defined as a measurement device that has completed a qualification process. The qualification process may include analyzing a shape of a signal received from the measurement device, determining configuration settings for the measurement device such that the shape of the signal will have predetermined characteristics, storing the configuration settings, and qualifying the measurement device as a verified device. If the measurement device is a verified device, the processor is programmed to configure the measurement device according to the configuration settings determined during the qualification process, receive a signal from the measurement device, and delivering the signal to the calculation model to determine the BP value of the user.

In some embodiments, a system for determining a BP value of a user includes a processor programmed to receive a signal from a sensor adapted to receive the signal from the user. The processor is further programmed to deliver the signal as an input to a model that returns the BP value as an output. In some examples, the model is developed according to a process that includes training the model using a first data set of signals and refining the trained model using a second data set of signals. In some examples, the signals of the second data set are annotated with information correlated with BP values collected using a reference methodology on a first cohort of subjects. The process for developing the model includes validating the refined model to ensure conformance with a validation criteria by comparing BP values returned by the refined model against BP values collected on a second cohort of subjects using the reference methodology.

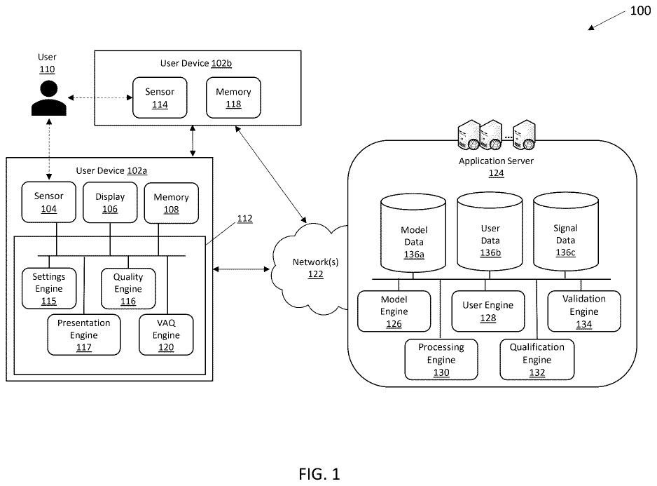

FIG. 1 is a block diagram of a BP measurement system 100 in accordance with aspects described herein. The system 100 includes a user device 102a and an application server 124. In some examples, the user device 102a is a smartphone, a computer, a tablet, a smartwatch, a wearable smart device, a non-wearable smart device, a smart device embedded within another item or device, or a dedicated medical device. Other types of user devices are possible. In some examples, user device 102a includes at least one sensor 104, at least one display 106, and at least one memory element 108. The user device 102a may have a housing that includes one or more sensors. The sensor 104 is configured to receive (or collect) a signal from a user 110. In some examples, the signal is a pulsatility signal. The pulsatility signal may be a PPG signal from a photoplethysmographic sensor, or any other pulsatility sensor or array of sensors, such as, but not limited to, a bioimpedance sensor, an ultra-sound sensor, a magnetic sensor, a radar sensor, a sensor based on radio frequency, a mechanical sensor, a volume sensor, a non-invasive sensor, an invasive sensor, an intra-arterial sensor, a minimal invasive sensor, a subcutaneous sensor, a tonometer sensor, a strain sensor, a plethysmographic sensor, a microphone, a capacitive sensor, an electromagnetic sensor, a Raman sensor, or any sensor capable of measuring a pulsatility signal either from a capillary bed of the skin or from any other section of the arterial tree. In some examples, the pulsatility signal is a unidimensional pulsatility signal. In some examples, the signal is derived from an image, a series of images, or a video captured by the sensor 104. The sensor 104 may alternatively be referred to as the camera 104.

In some examples, the system 100 includes a second user device 102b. The second user device 120b may differ from the first user device 102a in both type and functionality. For example, the second user device 102b may be a wearable bracelet, a band, a ring, a smartwatch, glasses, a bed sensor, or an integrated steering wheel sensor. Other types of user devices are possible. In some examples, the second user device 102b includes at least one second sensor 114 and at least one memory element 118. While not shown, the second user device 102b may include a display. It should be appreciated that the second user device 102b (or the second sensor 114) may be, or integrated within, any device, item, or object that is in continuous or close contact with the user's body. Examples of such include clothing, sensors embedded in clothing, sensors embedded on the user's house, mirrors, shoes, hats, glasses, smartphones, smartphone accessories, personal computers, webcams, cameras, computer accessories, tablets, cases, sockets, socks, hearing aids, jewelry, headphones, earbuds, gaming headsets, virtual reality headsets, smart glasses, bracelets, or any other suitable device, item, or object.

In some examples, the second sensor 114 is a different type of sensor than the first sensor 104; however, in other examples, the first and second sensors 104, 114 may be the same type of sensor. In some examples, the second sensor 114 is configured to receive (or collect) a pulsatility signal from the user 110. The pulsatility signal may be a PPG signal from a photoplethysmographic sensor, or any other pulsatility sensor or array of sensors, such as, but not limited to, a bioimpedance sensor, an ultra-sound sensor, a magnetic sensor, a radar sensor, a sensor based on radio frequency, a mechanical sensor, a volume sensor, a non-invasive sensor, an invasive sensor, an intra-arterial sensor, a minimal invasive sensor, a subcutaneous sensor, a tonometer sensor, a strain sensor, a plethysmographic sensor, a microphone, a capacitive sensor, an electromagnetic sensor, a Raman sensor, or any sensor capable of measuring a pulsatility signal either from a capillary bed of the skin or from any other section of the arterial tree. In some examples, the pulsatility signal is a unidimensional pulsatility signal. In some examples, the signal is derived from an image, a series of images, or a video captured by the second sensor 114.

In some examples, a client application 112 is configured to run (or operate) on the user device 102a. The client application 112 may be configured to run in a web browser or a special-purpose software application executing on the user device 102a (e.g., a smartphone application). In some examples, the client application 112 includes a settings engine 115, a quality engine 116, a presentation engine 117, and a video acquisition (VAQ) engine 120. The client application 112 is configured to process, at least in part, the signal received by the sensor 104 to determine BP values of the user 110. The user device 102a (or the client application 112) is configured to communicate with the application server 124 through one or more data communication networks 122 such as the Internet, for example. The client application 112 may communicate with the application server 124 over the network 122 using Hypertext Transfer Protocol (HTTP), another standard protocol, or a proprietary protocol, for example. The application server 124 provides functionality for processing data (e.g., pulsatility signals) and storing data (e.g., user data, signal data, etc.).

The application server 124 comprises software components and databases that can be deployed at one or more data centers (not shown) in one or more geographic locations, for example. The application server 124 software components include a model engine 126, a user engine 128, a processing engine 130, a qualification engine 132, and a validation engine 134. The software components can comprise subcomponents that can execute on the same or on a different individual data processing apparatus. The application server 124 databases include a model data database 136a, a user data database 136b, and a signal data database 136c. The databases can reside in one or more physical storage systems. Example features of the software components and data processing apparatus will be further described below. It should be appreciated that the application server 124 may include or otherwise support additional engines and tools. For example, the application server 124 may support third-party tools that provide additional functions to the engines 126-134 and/or the databases 136a-c. In some examples, the application server 124 is configured to utilize one or more Application Programming Interfaces (APIs) to communicate with third-party tools and engines.

In some examples, the second sensor 114 (or the second user device 102b) is configured to communicate with the client application 112 of the first user device 102a (e.g., over a wireless connection, Bluetooth, etc.). In such examples, the client application 112 may process, at least in part, signals received by the second sensor 114 to determine BP values of the user 110. The first user device 102a (or the client application 112) may then direct data, signals, and/or BP values associated with the second sensor 114 (or the second user device 102b) to the application server 124 via the network 122. In some examples, the second sensor 114 (or the second user device 102b) is configured to communicate directly with the application server 124 (e.g., via the network 122).

It should be appreciated that the client application 112 may be configured to run (or operate) on the second user device 102b. In such cases, the client application 112 may run on both the first and second user devices 102a, 102b or just the second user device 102b. Likewise, while not shown, the client application 112 may be configured to run (or operate) on other devices (e.g., a personal computer, tablet, etc.). In some examples, the client application 112 is independent of any specific device and is accessed via a web browser. For example, if the first user device 102a is a public device (or a third party device) and the second user device 102b is a wearable device without a screen, the user 110 may access the client application 112 via a web browser on their personal computer or another device (e.g., smartphone, tablet, etc.).

Although this application will describe many functions as being performed by client application 112, in various implementations, some or all functions performed by the client application 112 may be performed remotely by the application server 124. Likewise, in various implementations, some or all functions performed by the application server 124 may be performed locally by the client application 112.

While the system 100 of FIG. 1 includes the first user device 102a and the second user device 102b, it should be appreciated that each device 102a, 102b may operate as a standalone BP monitoring device that interfaces with the application server 124. In other words, different variations of the system 100 may include only one of the first user device 102a or the second user device 102b.

In some examples, the system 100 is designed to function as a certified and regulated medical device, software as a medical device (SaMD), or medical device software (MDSW), depending on the applicable regulatory framework in at least one jurisdiction. Given the critical role of accurate BP estimation in clinical decision-making, the system 100 may adhere to the specific medical device classification requirements, safety standards, and performance criteria set forth by regulatory authorities. Compliance with such regulations ensures that the system 100 meets predefined safety, effectiveness, and reliability thresholds, reducing risks associated with misdiagnosis or improper clinical use. By structuring the system's validation, risk assessment, and quality control measures to align with these global regulatory frameworks, the system 100 ensures broad international acceptance, allowing its deployment across multiple healthcare markets while maintaining stringent safety and efficacy requirements in accordance with the laws governing medical devices in different jurisdictions.

In some examples, the client application 112 functions as the primary display. The client application 112 may be validated and certified through rigorous regulatory approval processes, ensuring it adheres to mandatory standards of clinical accuracy, data integrity, real-time responsiveness, and usability required for direct clinical or therapeutic decision-making. When BP data calculated by the BP estimation model is delivered to users through the client application 112 as the primary display, the interface ensures immediate visibility and clinical interpretability of each measurement, thereby guaranteeing the accuracy, safety, and clinical relevance of the provided BP values. In such examples, client application 112 is directly accountable for patient safety and clinical reliability, offering users a regulated and standardized environment for cardiovascular monitoring.

In some examples, third-party applications may operate as optional displays (e.g., on the user devices 102a, 102b). The information delivered through optional displays may include historical summaries, trends, wellness insights, or aggregated cardiovascular data, rather than real-time, clinically actionable measurements intended for direct clinical use. This hierarchical delineation ensures compliance with regulatory frameworks by allowing third-party apps, operating as optional displays, to avoid triggering intensive regulatory scrutiny or stringent requirements, facilitating smoother integration into broader consumer wellness or lifestyle platforms.

This distinction between primary and optional display responsibilities confers multiple commercial, clinical, and user experience advantages. By clearly defining the client application 112 as the primary clinical display, strict regulatory control and compliance over critical cardiovascular measurements and clinical claims can be maintained. Simultaneously, third-party manufacturers can readily incorporate accurate, medical-grade BP data within their own platforms as optional displays without enduring extensive regulatory burdens or delays. The BP estimation model (e.g., of the model engine 126) further enhances this advantage by efficiently routing validated BP data streams to different user-facing interfaces-primary and optional-based on regulatory classification and clinical purpose. This flexibility enables medical-grade cardiovascular services to be extended across numerous third-party digital ecosystems while consistently ensuring the highest standards of clinical reliability, accuracy, and safety.

In some examples, the cloud-based BP estimation model's capability to selectively expose user-specific BP data to primary and optional displays also enhances user engagement and experience. For example, users may receive clinically validated, real-time measurements directly through the client application 112, ensuring immediate, trustworthy feedback necessary for medical decision-making. Simultaneously, the complementary, flexible access provided by optional displays within third-party wellness apps encourages ongoing user interaction, long-term health tracking, and personalized wellness guidance without compromising regulatory compliance. Users benefit from a unified cardiovascular monitoring experience that seamlessly integrates rigorous medical accuracy provided through the primary interface with comprehensive wellness-oriented features offered by partner platforms acting as optional displays.

### Measurement Process

A video acquisition system of the user device 102a is configured to capture pulsatility signals from the user 110. In some examples, the pulsatility signal is an arterial pulsatility. An arterial pulsatility signal refers to a physiological waveform captured by one or multiple sensors that reflects the rhythmic expansion and contraction of arteries or capillary beds due to the cardiac cycle. The signal contains rich information about the dynamic changes in blood volume or pressure within the vascular system, typically synchronized with each heartbeat. Depending on the sensor type and placement (e.g., PPG, pressure transducers, or ultrasound), these signals can originate from central or peripheral sites and may represent pulsatility in large arteries, smaller arterioles, or even capillary beds.

In some examples, the video acquisition system 100 includes the sensor 104 of the user device 102 and the VAQ engine 120 of the client application 112. The VAQ engine 120 is configured to control and operate the sensor 104 to capture the signals from the user 110. The VAQ engine 120 may use native software functions of an operating system of the device 102a to control and operate the sensor 104. In some examples, the video acquisition system includes the display 106 of the user device 102a. The display 106 may be used to view (or preview) the images or video being recorded to capture the signals. In some examples, a preview is generated by transforming the raw video into synthetic video that better represents to the user 110 the pulsatility signal, or assists the user 110 in better using the system 100 to improve the quality of the pulsatility signal. The video acquisition system is used to capture signals from a particular region of the user's body (e.g., a fingertip). In some examples, the sensor 104 is held in proximity to the region or pressed against the region to capture signals from the user 110. In some examples, the sensor 104 is configured to capture signals from the user 110 without contacting the user's body (e.g., by capturing an image/video of the user's face, body, etc.).

The video acquisition system of the user device 102a is configured to function effectively with or without artificial lighting capabilities. In environments where ambient lighting is sufficient, the sensor 104 can capture high-quality pulsatile signals from the user 110 (e.g., via the user's fingertip) without additional illumination. This adaptability ensures that users can obtain accurate blood pressure measurements in a variety of settings, whether indoors under artificial light or outdoors in natural daylight, enhancing the convenience and flexibility of the system 100. In scenarios where ambient lighting is inadequate, the system 100 may leverage a built-in flash light of the user device 102a as an artificial lighting source (e.g., the camera flash source). By illuminating the user's fingertip during the recording process, the flash light enhances the discernibility of blood flow characteristics captured by the sensor 104. In some examples, the client application 112 is configured to control the flash intensity, focal point of the flash, and timing to optimize signal quality while ensuring user comfort and safety, thereby enabling precise blood pressure measurements even in low-light conditions. It should be appreciated that the flash light of the user device 102a may be used during any scenario to enhance measurement collection.

In some examples, the sensor 104 includes a front-facing camera (e.g., camera 104a). When utilizing front camera modules for signal acquisition, the client application 112 may employ the display 106 of the user device 102a as an artificial lighting source by adjusting its brightness, and focal point-either partially or completely. By increasing screen luminance, the user's fingertip may be uniformly illuminated when placed over the front camera modules, improving the detection of pulsatile signals. This method eliminates the need for external light sources and allows for a user-friendly experience, as the screen's brightness can be modulated to balance signal quality with power consumption and user comfort.

The video acquisition system may be compatible with other artificial lighting sources beyond the native capabilities of the user device 102a. Such sources may include external devices, such as clip-on LED lights, ring lights, or specialized cases with integrated illumination designed to enhance sensor performance. By supporting a variety of lighting accessories, the system 100 ensures that users can achieve optimal signal quality under diverse conditions and with different device models, thereby expanding the usability and accessibility of the blood pressure measurement feature.

Various user devices, such as smartphones, may exhibit diverse configurations of back camera and flash distributions, which the system 100 accommodates to optimize performance.

For example, certain smartphone models feature a single, dual, or triple-camera setup with the flash positioned between or adjacent to the lenses. Likewise, some smartphone models include multiple flash modules. The VAQ engine 120 provides tailored guidance for each device configuration. For example, the VAQ engine 120 may provide visual content and instructions (e.g., via the display 106) to ensure that users correctly position their fingertip over the camera and flash components. In some examples, the VAQ engine 120 provides visual feedback to the user 110 to correct the positioning of their fingertip with respect to the camera(s) 104. In some examples, the VAQ engine 120 is configured to provide audio content to the user 110 such that the user 110 can receive instructions and feedback without needing to view the display 106. By accounting for these variations, the VAQ engine 120 maintains consistent signal quality across different devices.

Front camera configurations also vary among smartphone models, influencing how screen illumination is utilized. For example, certain smartphone models integrate the front camera alongside sensors within the display (or screen) area, allowing for targeted brightness adjustments in that region. Other devices feature punch-hole or under-display cameras, requiring different screen illumination strategies. The VAQ engine 120 is configured to adapt to these variations by modulating screen brightness appropriately, ensuring effective fingertip illumination and high-quality signal capture regardless of the front camera arrangement.

Some smartphones have a camera module that protrudes from the device's back surface, creating a physical protuberance. This design element can affect how the user's fingertip interfaces with the camera and flash during measurement. The VAQ engine 120 addresses this by providing specific instructions and visual aids to help users properly position their fingertip over protruding camera modules. For example, the VAQ engine 120 may recommend adjusting the finger angle or applying gentle pressure to ensure full contact, thus mitigating potential challenges posed by the device's physical characteristics.

Camera lens configurations, such as lens diameter and the presence of multiple lenses, can influence the area of contact and the quality of the captured signals. Larger lenses may allow for greater contact area and improved signal acquisition, while smaller lenses require more precise fingertip placement. The VAQ engine 120 incorporates adaptive algorithms that adjust to the lens specifications of each smartphone model. It provides customized user guidance to accommodate single, dual, or multi-lens setups, ensuring that the pulsatile signals are consistently and accurately captured across different camera configurations.

By integrating examples of various user device designs and their respective features, the VAQ engine 120 provides a high level of compatibility and robustness. Whether dealing with an devices equipped with a triple-lens rear camera and a true-tone flash, or a device featuring a high-resolution front camera with edge-to-edge display illumination, the VAQ engine 120 can adapt its operation accordingly. As such, the VAQ engine 120 (and the system 100) can provide precise blood pressure measurements across a wide spectrum of devices, accommodating differences in hardware layouts, sensor technologies, and user interaction modalities.

While some of the above examples describe the user device 102a as a smartphone, it should be appreciated that the user device 102a may be a different type of device. For example, the user device 102a may be any device equipped with a digital camera and sufficient processing capabilities, such as tablets, smartwatches, laptops, or dedicated medical devices. This versatility allows for a wide range of applications, extending the benefits of non-invasive blood pressure monitoring to various platforms.

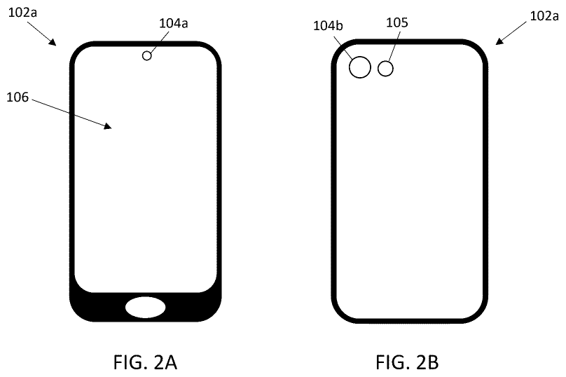

FIGS. 2A, 2B illustrate an example user device 102a that is used to capture measurements in accordance with aspects described herein. FIG. 2A illustrates a front view of the user device 102a showing the display 106 and a front camera 104a (i.e., a front-facing camera). FIG. 2B illustrates a back view of the user device 106 showing a rear camera 104a (i.e., a rear-facing camera) and a flash module 105. In some examples, the measurement process is performed by having a part of the user's body cover the lens of the camera 104a or 104b (e.g., by instance putting the fingertip on top of the camera). As will be described below, the measurement process may be performed in both display-side up and display-side down configurations.

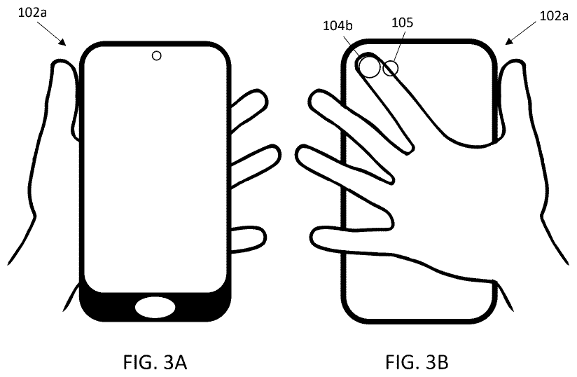

FIGS. 3A, 3B illustrate a first measurement configuration in accordance with aspects herein. As shown, the user device 102a is held in the hand of the user 110 display-side up. The user's finger (e.g., index finger) is positioned on top of the rear camera 104b. In some examples, the user's finger is positioned such that it does not cover the flash module 105. In some examples, the user's finger may partially cover or fully cover the flash module 105. In some examples, the flash module 105 is turned on during the measurement process (i.e., while the camera 104b is collecting images/video). In some examples, the flash module 105 is turned off during the measurement process. In some examples, the flash module 105 is turned on and off during the measurement process.

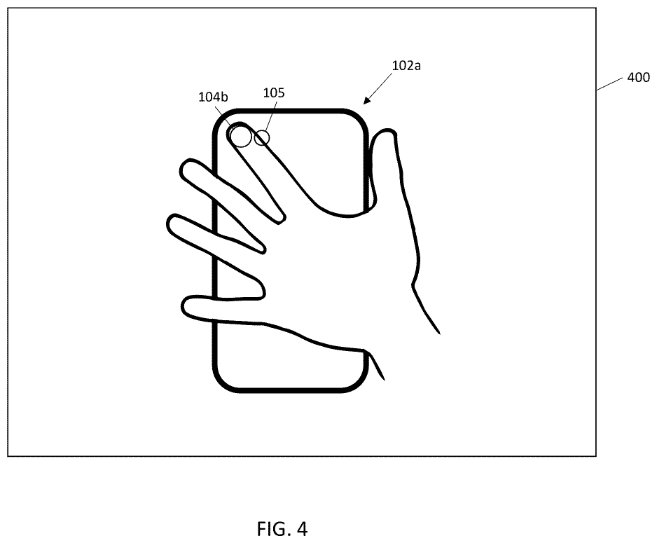

FIG. 4 illustrates a second measurement configuration in accordance with aspects herein. As shown, the user device 102a is placed display-side down on a flat surface 400. The surface 400 may be a table, countertop, or any other flat surface. The user's finger is positioned on top of the rear camera 104b. In some examples, the user's finger is positioned such that it does not cover the flash module 105. In some examples, the user's finger may partially cover or fully cover the flash module 105. In some examples, the flash module 105 is turned on during the measurement process (i.e., while the camera 104b is collecting images/video). In some examples, the flash module 105 is turned off during the measurement process. In some examples, the flash module 105 is turned on and off during the measurement process. This orientation provides a stable and secure setup for the measurement process, minimizing the potential for device movement that could interfere with signal acquisition. By having the display face down, the user 110 can comfortably position their fingertip over the lens of the rear camera 104b and the flash module 105 without needing to hold the device 102a, thereby reducing hand tremors or unintentional movements. This hands-free approach enhances the consistency and reliability of the measurements by ensuring that the device 102a remains stationary throughout the procedure.

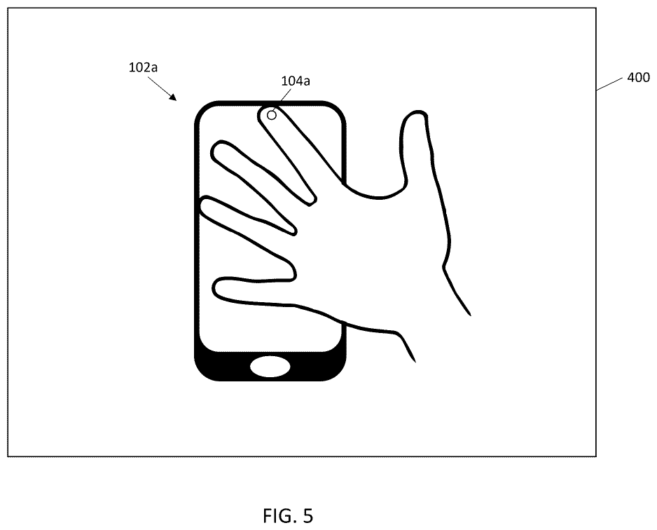

FIG. 5 illustrates a third measurement configuration in accordance with aspects herein. As shown, the user device 102a is placed display-side up on the flat surface 400. The user's finger is positioned on top of the front camera 104a. In some examples, the user's finger (or hand) is positioned such that it does not cover the display 106. In some examples, the user's finger (or hand) may partially cover or fully cover the display 106. In some examples, the display 106 is illuminated (or brightened) during the measurement process (i.e., while the camera 104a is collecting images/video). In some examples, the display 106 is turned off (or darkened) during the measurement process. In some examples, parameters (or content) of the display 106 are varied during the measurement process. For example, the brightness of the display 106 or the colors displayed may be varied during the measurement process. In some examples, specific regions of the display 106 are varied (e.g., in brightness or color). In some examples, the parameters, characteristics, and/or content of the display 106 are varied such that the user's finger is sufficiently illuminated in order to achieve a desired quality of the signals received from the user 110. In some examples, the quality engine 116 is configured to adapt the parameters, characteristics, and/or content of the display 106 in a real-time feedback loop. In some examples, the display 106 is used to present visual cues (e.g., text pop-ups, arrows and indicators) to guide the user 100 in positioning their finger in the right location. In some examples, the display 106 is used to present game-like content to obtain optimum pressure, color changing feedback (e.g., for pressure), and/or real-time camera feedback such that the user 100 can see which camera to put their finger on, if it's well placed, etc.

In some examples, measurement process incudes using the camera 104 to acquire a series of the images (or video) without contacting the user's body. For example, the camera 104 may be used to film a video of the user's entire face, a part of the face, the entire body, and/or part of the body. In such examples, the camera 104 records a series of images of the user 110 (e.g., the entire face, part of the face, the entire body, or a part of the body) from which the signal is extracted. In some examples, the measurement process leverages remote PPG (rPPG), where a smartphone's front camera captures subtle color variations in facial skin to extract pulsatility signals without direct contact. Expanding further, laptop or desktop webcams can perform facial rPPG, continuously tracking color fluctuations in the skin to estimate blood pressure passively during computer use.

Video-based BP monitoring can also be integrated into a smart mirror, where an embedded camera tracks facial blood flow changes, allowing the user 110 to measure their BP without direct interaction. In some examples, rPPG capabilities can be integrated into smart TVs, home assistants, or entertainment systems. In wearable applications, a smart headset, augmented reality (AR) headset, or smart glasses may feature a small camera that tracks subtle blood flow changes in the forehead or temple, offering continuous, non-invasive BP monitoring. Another application involves in-vehicle BP monitoring, where a dashboard camera tracks facial blood flow patterns using rPPG to provide real-time blood pressure measurements for drivers. This ensures safety by detecting potential hypertension-related risks during long trips. Additionally, security camera systems with AI-based health monitoring may be deployed in public spaces, passively tracking facial blood flow variations to detect hypertension in individuals entering hospitals, workplaces, or airports.

In some examples, AI-enhanced contact PPG acquisition in smart devices can improve accuracy through noise reduction, motion compensation, and artifact detection. Multi-camera systems can further enhance BP estimation by capturing synchronized PPG data from different parts of the face or body, combining multiple inputs for improved measurement reliability. IoT-connected video acquisition systems integrated into smart home environments can continuously record facial PPG data and transmit BP measurements to cloud-based health management platforms for remote monitoring. Professional-grade medical imaging systems in clinical settings could also employ video-based rPPG technology to monitor blood pressure in patients undergoing surgery or intensive care, eliminating the need for continuous cuff inflation. Similarly, public health kiosks equipped with video acquisition systems can enable walk-in BP monitoring by capturing facial rPPG signals, providing a seamless, contactless alternative to traditional cuff-based measurements.

These various techniques demonstrate the broad applicability of video acquisition systems for BP monitoring, extending beyond conventional wearables to include smartphones, tablets, computers, smart home devices, vehicle dashboards, security cameras, and clinical imaging systems. By integrating rPPG and contact-based PPG methods, such techniques make BP monitoring more accessible, seamless, and adaptable for both consumer and medical applications.

PPG technology can be implemented in different configurations depending on the sensor placement and intended use. The reflective PPG configuration is commonly found in smartwatches and wrist-worn devices, where the optical sensor is positioned against the skin and reflects light off the blood vessels beneath the surface. The transmissive PPG configuration, used in pulse oximeters and clip-on sensors for the fingertip or earlobe, transmits light through the tissue, measuring the amount of absorption by blood. In both cases, multi-wavelength PPG, utilizing green, red, and infrared light, can improve signal robustness and extract additional physiological parameters.

PPG sensors used for physiological signal monitoring come in various designs, each tailored for specific applications. PPG sensors can be categorized based on their wavelength, geometry, and chipset technology. The most commonly used wavelengths in PPG sensors are green (e.g., 500-570 nm), red (e.g., 600-700 nm), and infrared (e.g., 800-950 nm). In some examples, green light is preferred in wrist-worn devices due to its superior signal penetration in superficial arteries and lower sensitivity to motion artifacts. Red and infrared PPG sensors are frequently used in fingertip, ear, and chest-mounted applications due to their deeper tissue penetration, making them effective for tracking signals in environments with varying skin tones and thicknesses. PPG sensors can adopt various optical configurations, including transmissive, reflective, and sideways illumination. In transmissive PPG, light passes through the tissue (e.g., fingertip or earlobe), and a detector on the opposite side captures variations in transmitted light. This method generally provides high-quality signals but requires specific sensor placements.

Reflective PPG, more commonly used in wearables, relies on light being emitted onto the skin and reflected back to a detector positioned adjacent to the emitter. Sideways illumination PPG can be found in specific designs where the sensor and emitter are placed at an angle to optimize penetration depth and reduce external interference.

In some examples, PPG sensors are provided in integrated chip packages that combine optical sensing with analog front-end (AFE) circuits for signal conditioning and processing. Such chipsets may incorporate photodiodes, LED drivers, signal amplification, and filtering, allowing for high-fidelity signal acquisition. Some PPG chipsets also feature ML-enhanced signal processing, multi-wavelength capability, and adaptive power control to optimize battery efficiency and signal accuracy. In some examples, the diversity in PPG sensor designs, wavelengths, and configurations enables system 100 to be configured across a wide range of devices, ensuring adaptability for different user conditions and hardware capabilities. This flexibility further strengthens the robustness of the calibration-free BP estimation methodology, as it allows for the integration of the system into various commercial and medical-grade devices.

In some examples, the measurement devices include different types of sensors in addition to, or instead of, PPG sensors.

Electrocardiogramsors measure the electrical activity of the heart by detecting the bioelectric signals generated during cardiac cycles. In wearable devices, ECG sensors may be integrated into chest straps, patches, or smartwatches with conductive electrodes that contact the skin. ECG sensors provide detailed information about heart rhythm and are essential for detecting arrhythmias and other cardiac conditions.

Peripheral Arterial Tone (PAT) technology measures pulsatile volume changes in peripheral arterial beds to assess autonomic nervous system function and cardiovascular health. Devices utilizing PAT sensors, such as specialized finger probes, apply a uniform pressure field and use specific wavelengths of light to detect arterial pulsations. This technology is employed in diagnosing conditions like sleep apnea and endothelial dysfunction.

Some wearable devices may incorporate flexible pressure sensors made from materials like synthetic graphene and silver composites. These sensors detect mechanical deformations caused by arterial pulsations when worn on the skin, such as at the wrist. They offer high sensitivity and flexibility, making them suitable for continuous pulse monitoring in daily activities.

Ballistocardiography (BCG) sensors detect the mechanical recoil of the body resulting from the ejection of blood with each heartbeat. BCG sensors may be integrated into mattresses or smart clothing to monitor cardiac activity without direct skin contact. They provide insights into cardiac output and arterial stiffness.

Seismocardiography (SCG) sensors measure the vibrations of the chest wall induced by cardiac activity. Wearable devices equipped with accelerometers or gyroscopes may capture these subtle movements, enabling the assessment of heart contractility and timing intervals. SCG is useful for monitoring heart health in ambulatory settings.

Near-Infrared Spectroscopy (NIRS) sensors utilize near-infrared light to measure tissue oxygenation and hemodynamics. When integrated into wearables like headbands or armbands, NIRS sensors can assess cerebral or muscular blood flow, providing information on vascular health and oxygen delivery.

Radar technology, for example millimeter-wave (mmWave) and Doppler radar sensors, can be used in wearable devices to detect arterial pulsatility without direct skin contact. These sensors emit electromagnetic waves that reflect off the skin and underlying tissues, capturing minute movements caused by blood flow. Radar-based sensors may be advantageous in scenarios where contact-based measurements may be impractical, such as for elderly patients or individuals with sensitive skin.

Ultrasound-based sensors employ high-frequency sound waves to measure blood flow velocity and arterial pulsatility. These sensors use Doppler ultrasound principles to detect variations in blood flow speed within superficial arteries. Wearable ultrasound sensors can be integrated into patches or wristbands to provide high-accuracy hemodynamic assessments. Ultrasound can penetrate deeper tissues, making it particularly useful for tracking changes in arterial compliance and vascular resistance.

Impedance Plethysmography (IPG) sensors measure changes in electrical impedance caused by blood volume fluctuations within arteries. In wearable devices, IPG electrodes are placed on the skin to apply a small alternating current and detect variations in tissue conductivity due to pulsatile blood flow. This method enables non-invasive monitoring of cardiovascular parameters and has been explored for estimating BP trends over time. IPG sensors may be integrated into wrist-worn wearables, chest patches, and armbands, providing an alternative to optical-based blood flow monitoring.

Electrical Impedance Tomography (EIT) sensors extend the principles of impedance plethysmography by reconstructing tomographic images of tissue conductivity changes. Wearable EIT systems use multiple electrodes positioned around a body segment, such as the arm or chest, to create a real-time electrical map of pulsatile blood flow. This method allows for deeper insights into vascular function and provides a more spatially resolved measurement of hemodynamic changes. EIT sensors are being developed for integration into medical wearables, offering an innovative approach to continuous BP monitoring without requiring direct optical or mechanical contact.

Laser-based implementations of optical sensors can enhance PPG signal quality by using laser Doppler flowmetry (LDF) to measure microvascular blood flow with high sensitivity. LDF sensors can be integrated into wearable patches or wristbands, offering superior motion artifact resistance compared to traditional LED-based PPG sensors. Another optical technique, laser speckle contrast imaging (LSCI), uses coherent laser light to track dynamic blood flow changes at the microvascular level, making it an advanced option for measuring arterial pulsatility in high-fidelity applications.

Raman spectroscopy involves the use of a laser-based system to detect molecular vibrations caused by the interaction of light with blood components. Raman spectroscopy can be integrated into smart rings or wristbands to measure blood flow characteristics at a deeper tissue level, offering a high-resolution alternative to conventional PPG. Similarly, near-infrared spectroscopy (NIRS), commonly used in brain oxygenation monitoring, can be adapted to measure hemoglobin concentration changes in peripheral arteries, providing an alternative means of estimating pulsatility signals.

Optical coherence tomography (OCT) can be employed in specialized skin-adherent sensors or wrist-mounted medical devices to obtain detailed cross-sectional imaging of arterial walls, allowing the detection of pulsatility changes in microvascular structures.

Photoacoustic imaging, combines pulsed laser light with ultrasonic detection to create a highly detailed profile of blood vessel dynamics, enabling wearable systems to capture arterial pulsatility with exceptional depth resolution.

Optical sensors can also be enhanced using micro-LED arrays embedded into flexible, skin-worn patches, capable of real-time, continuous monitoring of blood flow patterns. These patches can adhere to the wrist, forearm, neck, or chest, providing versatile measurement locations optimized for different arterial beds. Some configurations integrate optical waveguides into textiles, allowing smart clothing to incorporate optical pulsatility sensors for non-invasive cardiovascular monitoring. Optical sensors can be combined with AI-enhanced signal processing to filter out artifacts from motion or environmental light variations, improving the accuracy of arterial pulsatility detection in real-world conditions.

The user device 102b is configured to capture pulsatility signals from the user 110. In some examples, the user device 102b is a wearable device equipped with one or more optical sensors (e.g., sensor 114) for measuring arterial pulsatility signals. Such wearable devices vary in form factor, body position, and functionality, offering multiple ways to integrate non-invasive BP monitoring into everyday life.

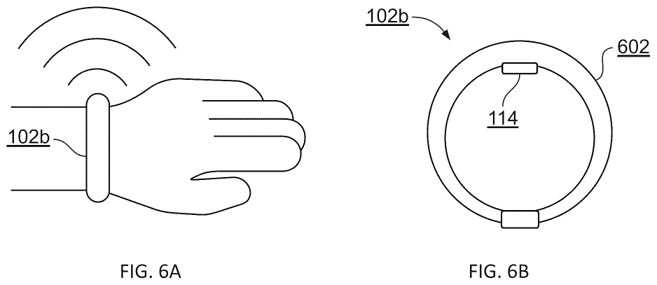

FIG. 6A illustrates a top view of an example user device 102b in accordance with aspects described herein. In some examples, the user device 102b is a wearable device (e.g., fitness tracker, medical device, etc.) that is worn on the wrist of the user 110. In some examples, the sensor 114 is a PPG sensor. The signals collected by the sensor 114 may be transmitted to the user device 102a and/or the application server 124. As shown in FIG. 6B, the sensor 114 of the user device 102b may be positioned on the inside of a strap 602 such that the sensor 114 contacts the user's skin while the device 102b is being worn. In some examples, sensor 114 is positioned such that it contacts the top portion of the user's wrist. In some examples, the sensor 114 is positioned such that it contacts a bottom (or back) portion of the user's wrist. Other sensor positions may be contemplated. It should be appreciated that the user device 102b may include two or more sensors (e. g, of the same type or different types).

In some examples, the user device 102b is a smartwatch with a reflective PPG sensor embedded on the underside of the device, continuously monitoring blood flow variations at the wrist. Fitness trackers and wristbands follow a similar approach, integrating multi-wavelength PPG sensors using green, red, and infrared LEDs to improve signal robustness across different skin tones and physiological conditions. Hybrid smartwatches, which blend traditional analog designs with optical health tracking, provide a balance between aesthetics and functionality. Medical-grade smartwatches incorporate FDA-or CE-approved optical sensors, ensuring clinical accuracy for regulated healthcare applications. Some wrist-worn devices also feature temperature-compensated PPG sensors, which adjust LED intensity based on skin temperature variations for improved measurement stability. AI-powered smartwatches and wristbands use adaptive learning algorithms to refine BP estimation over time, compensating for variations in user physiology and environmental conditions. Some multi-sensor hybrid wearables combine optical PPG with ECG, bioimpedance, or ultrasound sensors, creating multi-modal BP monitoring systems that enhance accuracy.

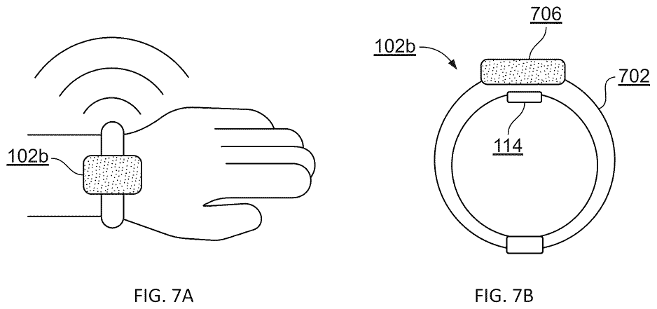

FIG. 7A illustrates a top view of another example user device 102b in accordance with aspects described herein. In some examples, the user device 102b is a wearable smartwatch device having a display 706 that is worn on the wrist of the user 110. In some examples, the sensor 114 is a PPG sensor. The signals collected by the sensor 114 may be transmitted to the user device 102a and/or the application server 124. As shown in FIG. 7B, the sensor 114 of the user device 102b may be positioned on the inside of a strap 702 (e.g., under the display 706) such that the sensor 114 contacts the user's skin while the device 102b is being worn. In some examples, sensor 114 is positioned such that it contacts the top portion of the user's wrist. In some examples, the sensor 114 is positioned such that it contacts a bottom (or back) portion of the user's wrist. Other sensor positions may be contemplated. It should be appreciated that the user device 102b may include two or more sensors (e. g, of the same type or different types).

In some examples, the user device 102b is a smart ring with an embedded infrared PPG sensor offers a discreet and compact way to capture pulsatile blood flow from the finger, where vascular structures are well-defined for accurate measurements. Some multi-sensor smart rings incorporate additional LEDs or micro-laser Doppler technology to improve signal depth and minimize motion artifacts. Fingertip PPG sensors, which function similarly to pulse oximeters but in a wearable format, can be used in specialized rings or clip-on devices for continuous tracking.

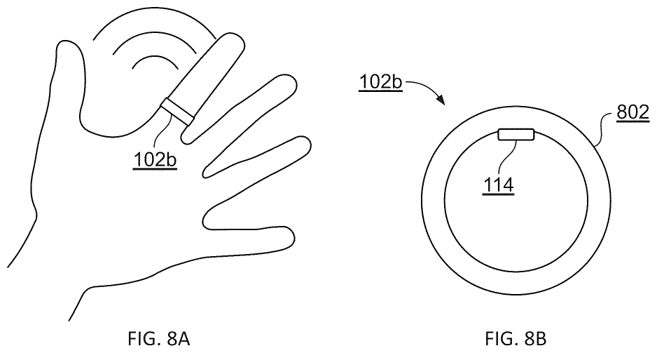

FIG. 8A illustrates a top view of another example user device 102b in accordance with aspects described herein. In some examples, the user device 102b is a wearable ring device (e.g., a smart ring) that is worn on a finger of the user 110. In some examples, the sensor 114 is a PPG sensor. The signals collected by the sensor 114 may be transmitted to the user device 102a and/or the application server 124. As shown in FIG. 8B, the sensor 114 of the user device 102b may be positioned on the inside of a band 802 such that the sensor 114 contacts the user's skin while the device 102b is being worn. In some examples, sensor 114 is positioned such that it contacts the top portion of the user's finger. In some examples, the sensor 114 is positioned such that it contacts a bottom (or back) portion of the user's finger. Other sensor positions may be contemplated. It should be appreciated that the user device 102b may include two or more sensors (e. g, of the same type or different types).

In some examples, the user device 102b is a pair of smart earbuds with optical PPG sensors. Earbuds provide an alternative to wrist-based monitoring by capturing arterial pulsations in the ear canal, where blood flow is less affected by movement. Smart hearing aids with integrated PPG sensors can serve dual purposes, catering to both cardiovascular health and auditory assistance. Smart glasses with built-in optical sensors in the temple region or earpieces can continuously track blood pressure through vascular structures in the forehead or sides of the head. These head-mounted systems are ideal for hands-free and passive BP monitoring, especially for professionals, athletes, or elderly users who require continuous tracking.

In some examples, the user device 102b is a chest-mounted wearable patch with flexible PPG sensors. Such patches can be designed for high-fidelity cardiovascular monitoring, particularly for medical and fitness applications. Chest-based monitoring may allow for recording, in addition to the pulsatility signal, the electrical activity of the heart (e.g., ECG). Such electrical activity may be used as input of the BP estimation module to enhance the accuracy of the BP reading. Smart clothing, such as biometric compression shirts, can integrate optical waveguides to deliver light-based pulsatility measurements across the chest or upper arm. Optical waveguide sensors integrated into textiles, such as compression sleeves, wristbands, or athletic wear, enable continuous blood pressure tracking without requiring direct contact with electronic hardware. These smart garments allow for passive, unobtrusive health monitoring over extended periods. Armband-style wearables, positioned on the upper arm like a blood pressure cuff but using optical sensors instead, provide an alternative location for measuring deep tissue blood flow, improving accuracy by minimizing motion-related artifacts. Wearable forehead patches or smart headbands with integrated multi-wavelength optical sensors enable high-sensitivity BP tracking from superficial temporal arteries. Smart sleep headbands designed for nighttime blood pressure monitoring use infrared-based PPG to collect pulsatility signals without disturbing the user's rest. Scalp-based optical sensors, potentially integrated into headwear like hats or helmets, can passively monitor BP while the user is engaged in daily activities. Flexible, tattoo-style optical sensors adhere to the skin and provide continuous real-time BP monitoring, particularly for medical-grade long-term tracking.

In some examples, the user device 102b is an ankle-worn PPG monitor. Such monitors may be designed for individuals with conditions such as peripheral artery disease (PAD), providing vascular health insights beyond conventional wrist-based wearables. These devices capture arterial waveforms from the dorsalis pedis or posterior tibial artery, areas typically assessed in clinical vascular examinations. Smart socks or foot-worn biometric patches could also integrate infrared PPG sensors to monitor vascular responses in the lower extremities, offering a novel application in cardiovascular research.

It should be appreciated that signals collected, processed, and analyzed by the system 100 can include non-pulsatile signals from the arteries of the user 110. Examples of non-pulsatile signals that may be captured from an artery of the user 110 include, but are not limited to, various imaging, mechanical, and optical modalities that do not rely on pulsatile blood flow. B-mode ultrasound imaging can provide static or time-averaged measurements of arterial diameter, wall thickness, and plaque morphology. Ultrasound-based elastography, including static or quasi-static techniques, enables the estimation of arterial stiffness or elasticity by analyzing tissue displacement or strain in response to external mechanical compression. Magnetic resonance imaging (MRI), including MR elastography, can yield high-resolution structural information such as wall thickness, lumen area, and local stiffness, independent of pulsatile dynamics. Optical coherence tomography (OCT) offers micron-scale imaging of arterial wall layers and structural composition, while photoacoustic imaging combines optical and acoustic contrast to assess wall structure, tissue composition, and stiffness. Electrical impedance tomography (EIT) allows estimation of arterial wall geometry and composition through conductivity mapping. Near-infrared spectroscopy (NIRS) can detect tissue composition and lipid content in arterial walls. Mechanical palpation or tonometry can be used to estimate arterial stiffness through controlled compression, and laser-based techniques such as laser Doppler vibrometry or laser speckle imaging can detect micromotions or surface vibrations of the vessel wall that are not associated with the cardiac pulse. In some examples, wearable sensors/devices are employed to capture non-pulsatile signals continuously and noninvasively. These may include strain or deformation sensors embedded in flexible substrates to detect changes in arterial geometry, wearable ultrasound patches capable of monitoring vessel wall motion or deformation without relying on pulsatile flow, optical sensors utilizing diffuse reflectance or NIRS for structural or compositional analysis, and piezoelectric or capacitive mechanical sensors that detect subtle arterial wall vibrations or stiffness during movement or external perturbation. Such techniques provide diverse non-pulsatile signal sources to assess arterial morphology, composition, and mechanical properties in both clinical and wearable monitoring contexts.

### Monitoring Process

To determine the user's BP, the user devices 102a, 102b transmit at least a portion of the data collected by the sensors 104, 114 to the application server 124 for processing. In some examples, the user device 102a is configured to transmit a single signal extracted from the frames captured by the sensor 104. For example, the client application 112 may extract a signal representing the entire frame to be transmitted to the application server 124. In some examples, the user device 102a transmits two or more signals extracted from the frames captured by the sensor 104. For example, the client application 112 may extract signals representing different regions of interest (ROIs) of each frame to be transmitted to the application server 124. In some examples, the user device 102a transmits a compressed version of video captured by the sensor 104 to the application server 124. In some examples, the user device 102a transmits raw video captured by the senor 104 to the application server 124.

The application server 124 is configured to manage user accounts securely and efficiently, forming the backbone of the system's cloud-based services. In some examples, the application server 124 employs a multi-tiered infrastructure that includes application-specific servers, database-specific servers, and authentication services. In some examples, load balancers distribute incoming traffic evenly across the servers to ensure high availability and scalability. The server architecture leverages secure APIs for communication between the client application 112 and the application server 124, utilizing encryption protocols such as TLS to protect data in transit. Firewalls and intrusion detection systems safeguard against unauthorized access, while user data is stored in encrypted databases (e.g., user data database 136b) with secure access controls. This robust architecture ensures seamless user account creation, authentication, and management, providing users with a reliable and secure experience.

Upon receiving the compressed pulsatility signals from the user devices 102a and 102b, the processing engine 130 performs advanced signal processing to prepare the data for blood pressure estimation via the model engine 126. In some examples, this involves aggregating multiple signals if available and conducting pre-processing steps such as de-noising, baseline correction, and normalization (e.g., via the processing engine 130). Techniques such as digital filtering may be applied to remove artifacts and enhance signal quality. The signals are then segmented into appropriate time windows, and feature extraction methods are used to identify key physiological markers relevant to blood pressure, such as pulse transit time, heart rate variability, and waveform morphology. This meticulous pre-processing ensures that the input to the predictive models is of high fidelity, enabling accurate and consistent blood pressure calculations by the model engine 126.

In some examples, each pulsatility signal captures at least one full heart cycle to ensure accurate and physiologically meaningful BP estimation. A heart cycle, which spans from one R-wave peak to the next in an electrocardiogram (ECG), represents one complete sequence of systole (i.e., heart contraction) and diastole (i.e., heart relaxation). During this cycle, arterial BP fluctuates in a characteristic waveform, and capturing a full cycle ensures that the system 100 acquires all relevant data points needed to extract clinically valid hemodynamic features.

One reason for processing a full heart cycle is that it allows the system 100 (or BP estimation model) to capture a complete transition between systolic and diastolic pressure. BP estimation depends on analyzing features such as peak systolic pressure, diastolic pressure, and pulse pressure amplitude. If the captured signal does not include an entire cycle, the model risks missing essential waveform components, leading to inaccurate estimations. Additionally, many BP estimation methods rely on techniques such as pulse transit time (PTT), pulse wave velocity (PWV), and pulse wave analysis (PWA), all of which rely on a complete waveform to generate a meaningful computation. While any of these techniques can be applied to pulsatility signals captured by the sensors/devices described herein, it may be desired to apply PWA techniques to PPG signals. PWA involves analyzing the waveform of a pulsatility signal to extract physiological information (e.g., BP). Without a full cycle, critical measurements such as peak-to-peak time differences, dicrotic notches, and waveform slopes cannot be reliably extracted.

Another consideration is heart rate variability. Since heart rate can fluctuate from resting levels of approximately 60 beats per minute to over 150 beats per minute during intense activity, capturing at least one heart cycle ensures that the system 100 adapts to different cardiovascular states. If less than a full cycle is captured, high heart rates may distort waveform interpretation, leading to inconsistent blood pressure readings. In addition, capturing an entire cycle allows for noise reduction and motion artifact correction. Physiological noise, including sensor displacement, movement artifacts, and respiration-induced variability, can distort the integrity of the waveform. A full cycle provides the necessary data points for signal processing techniques to detect and correct these distortions, ensuring accurate and reliable BP estimation.

If more than one heart cycle is available, additional computational strategies can be employed to enhance measurement accuracy. In some examples, a multiple cycle average is performed, where BP values are computed for each individual cycle and then averaged to reduce random noise and eliminate outliers. This may be useful in cases where motion artifacts or transient physiological changes affect individual waveform segments. In some examples, a trend detection and stability analysis is performed, where monitoring waveform consistency across multiple cycles helps identify anomalies such as arrhythmias or irregular pulse activity. This enables the system 100 to reject erratic readings and ensure that BP estimations are derived from stable and physiologically representative signals.

In some examples, when multiple heart cycles are captured, the system 100 can implement adaptive selection, where algorithms choose the most stable and noise-free cycles for computation. This may be beneficial in wearable devices (e.g., user device 102b) where users move frequently, introducing transient distortions in the recorded signal. The system 100 can also dynamically adjust the number of cycles analyzed based on heart rate. At lower heart rates, multiple cycles may be used to compensate for slow pulsatility changes, whereas at higher heart rates, a single or two consecutive cycles may be sufficient to ensure a rapid and accurate estimation.

In some examples, longitudinal analysis across multiple cycles enables real-time monitoring of BP trends, particularly in ambulatory settings where BP fluctuates due to physical activity, stress, or circadian rhythms. Capturing multiple cycles allows the system 100 to integrate heart rate variability metrics, which are known to be correlated with BP fluctuations, providing additional physiological context to the readings. In devices where an ECG signal is available, synchronizing PPG-based BP measurements with ECG-detected R-wave peaks further improves accuracy and robustness.

In some examples, machine learning (ML) models can be used to leverage multiple heart cycles to improve pattern recognition and refine blood pressure predictions. Likewise, BP estimation model can train on multi-cycle data to compensate for transient physiological variations, stress-induced BP fluctuations, or hydration effects. By continuously learning from multiple readings, these models improve the accuracy of long-term blood pressure monitoring. Additionally, capturing multiple heart cycles can aid in detecting arrhythmias, skipped beats, or irregular pulse activity, offering additional clinical insights beyond simple blood pressure estimation.

Regulatory considerations also favor the inclusion of multiple heart cycles in BP estimation. Medical device validation protocols, such as ISO 81060-2, IEEE 1708, and AAMI/ESH standards, require validation across multiple measurements to ensure accuracy and reproducibility. In some examples, capturing more than one cycle allows the system 100 to meet regulatory requirements and increases confidence in the clinical reliability of the device. Ensuring that at least one full heart cycle is captured provides a foundation for reliable BP estimation, while incorporating multiple cycles, when available, enhances accuracy, noise resilience, and long-term trend analysis. These techniques are particularly valuable for wearable devices, consumer health applications, and medical-grade monitoring solutions, ensuring that BP estimation remains robust, physiologically meaningful, and clinically valid across diverse user conditions.

### BP Estimation Model

The blood pressure estimation by the model engine 126 relies on a sophisticated model architecture developed using ML techniques tailored for physiological signal analysis. In some examples, the model architecture consists of deep neural networks with layers optimized for temporal and spatial pattern recognition, such as convolutional and recurrent neural networks. The models are trained on large datasets to capture complex relationships between the pulsatility signals and corresponding blood pressure values. In some examples, the training datasets are stored in the model data database 136a. Regularization methods and cross-validation are employed to prevent overfitting and to enhance generalizability. The architecture is designed to be modular, allowing for updates and improvements as new data becomes available, and to accommodate different input types and configurations.

In some examples, the model engine 126 maintains a comprehensive database that stores annotated signals (e.g., the signal data database 136c), where each pulsatility signal is linked with corresponding blood pressure measurements and metadata such as time stamps, user demographics, and environmental conditions. These annotations enable supervised learning, allowing the predictive models to learn accurate mappings between input signals and blood pressure value outputs. The annotated dataset serves as a valuable resource for refining the predictive algorithms, identifying patterns, and improving model robustness. It supports ongoing research and development efforts aimed at enhancing the system's capability to deliver precise and personalized blood pressure estimates.

In addition to annotated data, the signal data database 136c may store non-annotated signals generated during routine use of the system 100. These signals, while not immediately linked to known blood pressure values, provide a rich source of information for unsupervised learning and exploratory data analysis. They help in identifying new patterns, understanding user behavior, and detecting trends that may not be apparent from annotated data alone. Incorporating non-annotated signals allows the system 100 to adapt to a wider range of physiological variations and environmental factors, ultimately contributing to the continuous improvement of the predictive models. The accumulation of both annotated and non-annotated signals enables the models to evolve and adapt, enhancing their accuracy and reliability over time. This iterative improvement process ensures that users receive increasingly precise blood pressure measurements, justifying any initial inconveniences and reinforcing the system's value proposition.

As described above, the BP estimation model may be located remotely from the sensor, and the sensor data is transmitted from the sensor/device to an external system-such as a smartphone (e.g., user device 102b), a cloud-based AI engine (e.g., model engine 126), or a dedicated server (e.g., the application server 124)—for BP estimation. This setup is ideal for computationally intensive models, where advanced deep learning architectures require high-performance cloud servers to process vast amounts of data. In a wellness-focused configuration, a smart ring or smartwatch with a PPG sensor (e.g., user device 102b) may transmit raw or pre-processed optical signals via a wireless connection to a companion app on a smartphone (e.g., user device 102a), where a cloud-connected BP estimation model performs inference and returns results to the user in seconds. This configuration allows the BP estimation model to continuously improve, as updates can be deployed remotely without requiring firmware changes in the wearable itself. In a medical-focused configuration, secure data transmission from a wearable sensor (e.g., user device 102b) to a hospital cloud infrastructure (e.g., application server 124) may be preferred. In one example configuration, a PPG-enabled wristband continuously sends signals to a central AI-based health monitoring platform, where readings are aggregated and analyzed in real-time for early hypertension detection. This configuration may be ideal for telemedicine platforms, where patients can wear a lightweight sensor, and doctors receive real-time BP assessments without requiring the patient to manually log or interpret the results.

While the BP estimation model has been described above as being part of the model engine 126 of the application server 124, it should be appreciated that the BP estimation model may be located on the user device (e.g., 102a or 102b). When the BP estimation model is located on the same device as the sensor, the system 100 may operate in a fully self-contained manner, leveraging edge computing to provide real-time BP estimation without requiring external connectivity (e.g., to the application server 124). This approach ensures low latency, enhanced user privacy, and continuous operation even in the absence of an internet connection. In such examples, the model may be integrated into a the user device's local processing unit. In some examples, an AI-powered on-device inference model is leveraged to process PPG signals locally, providing users with instantaneous BP readings displayed on the device's screen. For example, a smartwatch with an embedded neural processing unit (NPU) may efficiently process PPG data using a pre-trained model, ensuring that the user 110 can continuously monitor BP trends throughout the day without cloud connectivity. Likewise, in medical applications, on-device modeling ensures regulatory compliance with data privacy laws such as GDPR and HIPAA by keeping sensitive biometric data on the user's device. As an example, an FDA-cleared smartwatch integrates dedicated medical-grade algorithms into its firmware, with rigorous on-device quality validation ensuring that every BP reading meets clinical accuracy standards before being displayed or stored. This approach benefits real-time continuous monitoring, where user devices collect data and instantly process it, making it ideal for users requiring immediate feedback without reliance on external systems.

In some examples, the processor is remotely located from the sensor, allowing distributed computing architectures to enhance flexibility in commercial implementations. In such examples, the sensor captures raw optical data, which is then transmitted to an external processor—such as a dedicated gateway device, a smartphone, or a cloud server—where computation is performed. This configuration may be useful in multi-device ecosystems, where the sensor is small and lightweight, optimized for continuous wearability, while the processing unit resides elsewhere. For example, a commercially viable wearable patch with a minimalistic form factor, designed for 24/7 blood pressure monitoring, may use Bluetooth Low Energy (BLE) transmission to send raw PPG data to a smartphone app (e.g., client application 112) that serves as the primary processing unit. In some examples, this setup extends battery life and allows for cross-device interoperability, where the same sensor can be paired with different computing platforms depending on user preferences or regulatory environments. Similarly, in a medical setting, a smart textile or medical-grade armband may send unprocessed biometric signals via Wi-Fi to a hospital server (e.g., application server 124), where advanced models perform real-time BP estimation. Such an implementation allows hospitals and research institutions to maintain full control over processing algorithms, updating them based on the latest medical insights without requiring hardware modifications. Remote processing also supports federated learning models, where anonymized data from multiple users can contribute to ongoing algorithm improvements while maintaining privacy protections.

### Calibration-Free BP Determination

As described above, many existing BP monitoring devices require initial and/or periodic calibrations with cumbersome reference devices, such as oscillometric cuffs. Such calibration requirements impose practical limitations, user inconvenience, and potential inaccuracies due to improper calibration. In some embodiments, a calibration-free BP determination system (e.g., system 100 of FIG. 1) is configured to use physiological signals that are non-invasively captured from the user 110 by sensors, such as one or more PPG sensors integrated within a camera (e.g., sensor 104 of user device 102a) or a wearable device (e.g., sensor 114 of user device 102b) without relying on initial and/or periodic calibrations (e.g., from a reference device). In some examples, the system 100 validates whether the sensors 104, 114 or the received signals meet predetermined quality or contextual criteria before allowing the signals to be processed by the BP estimation model (e.g., of the model engine 126 of the application server 124). This validation process ensures that only high-quality signals (e.g., those meeting rigorous standards of physiological plausibility, integrity, and regulatory compliance) are utilized, thus guaranteeing clinical accuracy and reliability.

By integrating the validation process, the system 100 can ensure the clinical accuracy and reliability of BP values independent of user-driven calibrations. In some examples, the validation process systematically evaluates whether the sensor 104 or 114, or the captured physiological signals, comply with predetermined criteria, including signal integrity, physiological plausibility, and regulatory requirements. Such rigorous criteria ensures that the data fed into the BP model of model engine 126 is consistently reliable and physiologically relevant. Consequently, the system 100 can confidently produce medically accurate and consistent BP readings under diverse real-world conditions, unlike existing BP monitoring devices that rely solely on basic artifact rejection or occasional user calibrations. Integration of the validation process enables the system 100 to minimize inaccuracies and inconsistent performance due to uncontrolled, noisy, or physiologically invalid signals. Likewise, by systematically filtering substandard data, the system 100 delivers consistently accurate BP values to the user 110, enhancing clinical utility and user confidence.

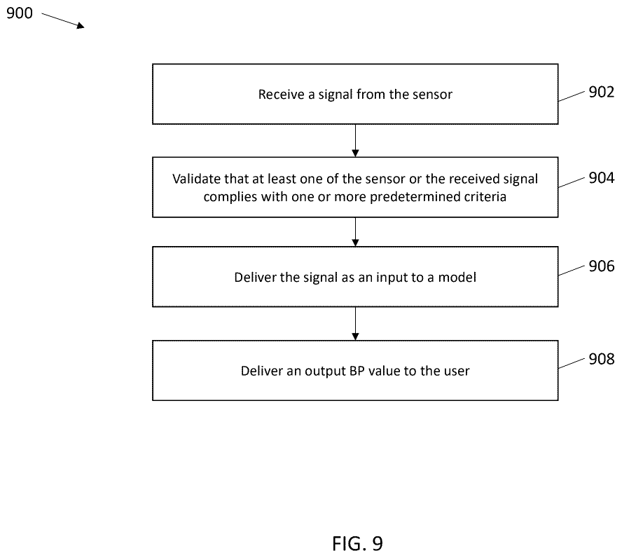

FIG. 9 illustrates a method 900 of operation for a calibration-free (or calibrationless) BP determination system in accordance with aspects described herein. In some examples, the system 100 of FIG. 1 is configured to perform the method 900. It should be appreciated that the method 900 may be performed using the first user device 102a and/or the second user device 102b.

At step 902, a signal is received by the validation engine 134 of the application server 124. In some examples, the signal is collected from the user 110 by sensor 104 or 114 and transmitted from user device 102a or 102b to the application server 124 (e.g., via the network 122). The signal may be a pulsatility signal, a physiological signal, or any other type of signal that is representative of data collected by the sensors 104, 114. In some examples, the signal is collected by a PPG sensor embedded in a wearable device such as a smartwatch, fitness band, ring, earbud, etc. In some examples, the signal is collected by an optical sensor in a smartphone, tablet, etc. For example, the signal may be collected using video-based physiological signal acquisition. In some examples, the user 110 places a fingertip over the rear camera of a smartphone (e.g., sensor 104 of user device 102a) while the built-in flash provides illumination. The camera records variations in light absorption due to blood volume changes, extracting PPG signals. Alternatively, the smartphone's front-facing camera may be used for contact-based PPG acquisition when the user 110 places their fingertip over the lens. In some examples, the signal is pre-processed by the processing engine 130 of the application server 124 before being received by the validation engine 134.

At step 904, the validation engine 134 validates that at least one of the sensor or the received signal complies with one or more predetermined criteria. In some examples, the validation engine 134 determines that the signal is valid if (i) the sensor 104 or 114 complies with predetermined sensor criteria or (ii) the signal complies with predetermined signal criteria. In some examples, the validation engine 134 determines that the signal is valid only if (i) the sensor 104 or 114 complies with predetermined sensor criteria and (ii) the signal complies with predetermined signal criteria. In some examples, the predetermined sensor criteria is applied to information related to the sensor 104 or 114. Such information may be transmitted with the signal to the application server 124 (e.g., as metadata). In some examples, the validation engine 134 is configured to query the user device 102a or 102b for sensor information in response to receiving the signal. In some examples, the validation engine 134 is configured to retrieve the sensor information from a database (e.g., the user database 136b). In such examples, the validation engine 134 may retrieve sensor information for a user device that is known to be associated with the user 110 (e.g., linked to a user account).

In some examples, the predetermined validation criteria enables the system to filter out erroneous signals, minimize noise artifacts, and ensure that only physiologically meaningful data contributes to BP prediction. Examples of validation criteria include metrics relating to signal integrity, such as, for example, signal-to-noise (SNR) ratio, waveform stability, and physiological plausibility. Signal integrity checks ensure that the raw signal maintains consistency and stability before it is processed. In some examples, the validation engine 134 performs an SNR assessment that evaluate the strength of the pulsatile component of the signal relative to background noise, ensuring the sensor effectively captures arterial pulsations. In some examples, baseline drift detection is applied to identify slow fluctuations in the signal baseline, which may indicate improper sensor placement, ambient light leakage, or temperature-induced sensor drift. In some examples, sudden signal amplitude spikes caused by motion artifacts or temporary loss of skin contact are monitored and filtered out to prevent waveform distortion.

In some examples, the validation engine 134 uses artifact detection techniques to further enhance the system's ability to recognize and discard distorted signals. In some examples, motion artifact detection utilizes accelerometers or gyroscopes in wearable devices (e.g., user device 102b) to detect movement that could introduce noise. When significant motion is detected, the system 100 may flag the corresponding signal as unreliable. Ambient light interference detection ensures that PPG sensors operating in uncontrolled lighting conditions, such as smartphone video-based acquisition, do not introduce erroneous signals. In some examples, skin contact consistency checks are used to validate that the optical sensor maintains continuous, stable contact with the skin, as disruptions can cause inconsistent signal readings.

In some examples, the validation engine 134 uses a physiological plausibility measurement to confirm whether the recorded signal falls within expected human physiological ranges. In some examples, a heart rate plausibility check is used to compare detected pulse rates against normative values, discarding signals that indicate implausible heart rates. In some examples, a pulse waveform morphology assessment is used to analyze whether the detected pulse wave follows a physiologically valid pattern, rejecting signals with excessive flattening or irregular peaks. In some examples, a dicrotic notch detection validation is used to ensure that the secondary inflection point, associated with aortic valve closure, is present in the expected position within the signal waveform. If absent or inconsistent, the signal may be flagged as unreliable.

In some examples, the validation criteria includes contextual information, such as, for example, verifying if the signal was collected under conditions suitable for accurate BP measurement (e.g., user posture, relaxation state, or a movement status collected through additional sensors like accelerometers or gyroscopes). The validation criteria may include confirming device and/or sensor compatibility and performance. Such criteria may indicate whether the device or sensor is capable of adhering to specific regulatory standards of territories where the BP values will be utilized. In some examples, the validation engine 134 employs both intrinsic signal validation, which analyzes the signal waveform characteristics, and contextual validation, which adapts signal assessment based on external conditions such as body position, time of day, physical activity, and regulatory compliance in the user's territory.

In some examples, the validation engine 134 is configured to perform a body position-based signal validation that ensures that the sensor captures an arterial pulsatility signal under physiologically appropriate conditions. For example, wearable devices (e.g., user device 102b) equipped with accelerometers and gyroscopes may detect whether the user is sitting, lying down, standing, or moving, and communicate this information with the validation engine 134. If the system 100 detects that the user 110 is standing or walking, it may flag the signal as unreliable or prompt the user 110 to stabilize before measurement. Likewise, wrist-worn devices may check whether the arm is relaxed at heart level, ensuring optimal arterial alignment for PPG-based blood pressure estimation. For sensors placed on the ear, chest, ankle, or finger, validation thresholds may be adjusted based on the expected perfusion characteristics of those body sites. An ankle-worn device, for example, must account for hydrostatic pressure variations, ensuring that posture-related vascular shifts do not skew BP estimations.

Given that time-of-day variations can influence cardiovascular physiology, the validation engine 134 may incorporate a circadian rhythm-based validation that improves measurement reliability. BP follows a diurnal cycle, typically peaking in the morning and decreasing at night. In some examples, the validation engine 134 integrates timestamp metadata and correlates detected BP values with expected time-based trends. If a nighttime measurement exhibits an unexpected spike, the validation engine 134 may prompt a revalidation step or suggest a follow-up reading. The validation engine 134 may also cross-reference ambient light sensor data or sleep tracking information from an external wearable to determine whether the user 110 is in a resting or active phase. A morning hypertension surge, commonly observed upon waking, may trigger adjusted validation parameters, ensuring that transient cardiovascular effects do not falsely inflate readings. Similarly, BP dips during an afternoon energy slump may be flagged for revalidation if they fall below expected physiological norms.

In some examples, the validation engine 134 used regulatory compliance-based signal validation to ensure that measurements conform to the standards of the user's geographic location. A wearable device (e.g., user device 102b) with GPS capabilities or smartphone-based location services (e.g., of user device 102a) can determine the user's country and adjust its validation parameters accordingly. Different regions impose distinct accuracy and reliability requirements for medical devices. In Europe (i.e., CE-marked devices), the system 100 may enforce stricter validation protocols based on ISO 81060-2, ensuring compliance with clinical validation standards. In the United States (i.e., FDA-regulated devices), additional verification steps may be used to align with AAMI SP10 standards or any relevant standards/guidance recognized by the FDA at the time of the measurement. If the user 110 travels between regulatory territories, the system 100 may dynamically adjust its signal validation mode or request manual confirmation of the preferred framework. Additionally, integration with electronic health records (EHR) or third-party health platforms allows the system 100 to ensure compliance before transmitting data for clinical use.

In some examples, physical activity-aware validation methods are used to prevent misleading BP estimates caused by transient cardiovascular fluctuations. A wearable device (e.g., user device 102b) equipped with motion sensors, gyroscopes, or heart rate monitors can determine whether the user 110 is at rest, walking, or exercising. If a high-intensity activity is detected, the validation engine 134 may reject the signal or prompt the user 110 to wait before reattempting measurement. If light physical activity is detected, the system 100 may apply motion-compensated filtering algorithms to stabilize the waveform. Additionally, external data sources such as smartphone step counters or fitness tracking apps can confirm whether the BP measurement occurred during or after exertion. If the validation engine 134 detects that a reading was taken immediately after exercise, it may delay validation until vascular tone stabilizes, preventing erroneously high estimates caused by transient exercise-induced hypertension.

In some examples, the validation engine 134 is configured to consider user-provided inputs or real-time contextual data. For example, a smartwatch linked to a smartphone may access weather data to determine whether external temperature conditions could affect vascular tone. If the system 100 detects cold exposure, which is known to cause peripheral vasoconstriction, it may raise the threshold for acceptable signal amplitude variations, ensuring that reduced perfusion does not lead to misclassification of the signal as low quality. Conversely, in high-temperature environments where vasodilation may occur, the system 100 may implement adaptive gain control to prevent overestimation of pulsatility signals. Integration with smart home systems or sleep trackers allows refinement of BP validation criteria based on circadian physiology, dynamically adjusting quality thresholds depending on whether the user 110 is waking up, preparing to sleep, or experiencing disrupted sleep cycles.

In some examples, the predetermined validation criteria corresponds to different medical device frameworks, such as, for example, the FDA (510k and PMA processes in the U.S.), EU MDR (CE Marking in Europe), NMPA (China), TGA (Australia), and PMDA (Japan). Such frameworks may impose specific accuracy and performance validation criteria for BP measurement devices. These frameworks may mandate that medical devices provide evidence of measurement consistency, robustness across diverse patient populations, and adherence to strict performance limits. For example, the ISO 81060-2 standard for BP monitors requires validation against reference auscultatory measurements, ensuring that the device maintains a bias of less than 5 mmHg and precision within 8 or 10 mmHg under controlled conditions. Similarly, the IEEE 1708 standard for wearable blood pressure devices defines acceptable measurement drift limits, ensuring long-term stability in real-world conditions. By implementing a sensor or signal validation step directly linked to regulatory compliance, the system 100 introduces a layer of control that prevents data corruption, measurement instability, and performance degradation over time. This validation mechanism may include real-time signal assessment, sensor qualification before integration, post-market quality control measures, or adaptive recalibration based on regulatory validation reports. If a device does not meet the predefined regulatory compliance threshold, the system 100 may reject the signal, prompt revalidation under controlled conditions, or adjust internal processing models to compensate for measurement inconsistencies.

It should be appreciated that the regulatory implications of the validation step extend beyond accuracy control and into risk management, a core requirement for medical device approval. Many regulatory agencies impose strict post-market surveillance (PMS) requirements, where manufacturers must demonstrate that device performance remains consistent after deployment. In some examples, the validation of sensors and signals ensures that calibration-free systems do not degrade over time, thereby satisfying ISO 14971 (risk management for medical devices) requirements. The validation step also facilitates regulatory submissions, as manufacturers can present real-world validation data that supports the device's ability to maintain clinical accuracy across different sensor types, user behaviors, and external conditions.

In some examples, the validation criteria is dynamically adjusted depending on local regulatory requirements, ensuring compliance in multiple jurisdictions without requiring extensive re-engineering of the system 100. For instance, if the system 100 detects that it is operating in the United States, it may enforce compliance with AAMI SP10 standards, while in Europe, it may prioritize ISO 81060-2 validation requirements. This territorial adaptability ensures that the system 100 remains a globally deployable, compliant medical device. In some examples, the adaptive compliance mechanism leverages extensive prior validation work, including clinical studies, bench tests, and rigorous regulatory evaluations, to establish clear relationships between predetermined sensor and signal quality criteria and the accuracy, reliability, and safety benchmarks set forth by local medical device regulations. Furthermore, this validation step enhances clinical acceptance and regulatory confidence by ensuring that calibration-free BP measurements are not only accurate in controlled environments, but also reliable in real-world applications. Unlike systems that rely on retrospective algorithm validation during regulatory approval, the system 100 continuously enforces validation during operation, effectively bridging the gap between regulatory requirements and real-world performance. This ensures that medical professionals, regulatory bodies, and end-users can trust the system's output, enabling wider adoption and seamless integration into digital health ecosystems.

Consequently, having established these rigorous sensor and signal quality requirements in pre-market validation studies and regulatory submissions, the system 100 may operationalize these findings in a real-world deployment scenario. By validating in real-time—at every measurement—that either the sensor itself or the signals it produces conform to these predetermined criteria, the system 100 can ensure continuous and automatic compliance with local regulatory standards. Thus, compliance moves from being a static, pre-market approval requirement into an active, real-time enforcement mechanism. For instance, if the device operates in a European jurisdiction subject to EU MDR, the validation step continuously enforces compliance with criteria aligned to ISO 81060-2, thereby guaranteeing the accuracy and reliability of every delivered BP reading within that jurisdiction. Conversely, when deployed in a U.S. regulatory context, the validation step dynamically adjusts to enforce adherence to criteria aligned with AAMI SP10 or any relevant standards/guidance recognized by the FDA at the time of the measurement, thereby ensuring regulatory compliance with FDA expectations. In addition, this adaptive mechanism facilitates efficient international scalability, enabling a single device (e.g., user device 102a, 102b) to dynamically adjust its regulatory compliance validation according to the user's geographic territory. This flexibility eliminates the need for developing separate device variants for different regulatory jurisdictions and significantly reduces the regulatory burden associated with entering new markets. Ultimately, the combination of rigorous pre-validation studies and real-time adaptive validation empowers the system 100 to provide sustained clinical accuracy, continuous regulatory compliance, and robust operational reliability across diverse global regulatory environments.

In some examples, the system 100 allows the user 110 to manually select the applicable regulatory framework via the client application 112 during initial device setup or in a settings menu. This may be particularly useful for users who frequently travel between regulatory territories or professionals operating in multiple healthcare environments where different compliance requirements apply. The client application 112 may present a list of regulatory frameworks, such as “FDA (United States),” “EU MDR (European Union),” “TGA (Australia),” “NMPA (China),” and “PMDA (Japan),” enabling the user 110 to select the most appropriate setting. In some examples, the system 100 may prompt the user 110 to confirm the applicable regulatory territory whenever they initiate a BP measurement for the first time in a new location. This mechanism ensures that the validation of the sensor or signal aligns with the requirements of the selected jurisdiction before generating a BP reading. In some examples, the system 100 provides detailed regulatory compliance information within the UI of the client application 112, explaining the differences between each framework and its implications for accuracy thresholds, validation methods, and potential clinical use.

In some examples, the system 100 adapts its regulatory framework based on a user profile of the user 110. For example, a general consumer may select a wellness mode that adheres to minimal regulatory constraints, whereas a clinician may select a medical-grade compliance mode that enforces stricter signal validation standards (i.e., a stricter validation criteria). This implementation ensures flexibility while maintaining regulatory integrity across different user types and use cases. In some examples, multi-user settings allow an organization or institution, such as a hospital or telemedicine provider, to configure validation criteria settings centrally and apply them across multiple connected devices. For example, an administrator may select the appropriate regulatory framework within the system's settings, ensuring uniform compliance for all users within a specific healthcare network. In some examples, the user 110 to selects regulatory preferences manually and the system 100 provides smart recommendations based on geolocation data or past selections. For example, if the user 110 previously selected “FDA compliance” while in the United States but has since traveled to Europe, the system 100 may suggest switching to “EU MDR compliance” while still allowing manual confirmation from the user 110.

In some examples, the system 100 automatically determines the applicable regulatory territory using GPS-based geolocation or IP address-based location detection from a connected smartphone or networked device (e.g., user device 102a). Upon detecting a new geographic region, the system 100 may seamlessly adjust its regulatory settings to comply with local medical device laws without requiring user intervention. In some examples, the system 100 leverages mobile network information to determine the user's country based on SIM card registration data or carrier metadata. In some examples, the user device 102a detects the applicable regulatory environment by retrieving the language and regional settings of the operating system or health platform it is integrated with, such as Apple HealthKit, Google Fit, or a hospital's electronic health record (EHR) system. If the user 110 is registered with a national healthcare provider, the system 100 may use this data to infer the applicable regulatory territory and apply the appropriate compliance settings. In some examples, the system 100 utilizes real-time network verification with the device manufacturer's cloud servers. Upon connecting to the internet, the system 100 cross-references its current geolocation with an updated regulatory compliance database hosted by the manufacturer. This ensures that even if regulatory requirements change, the system 100 dynamically updates itself to maintain full compliance. In some examples, the system 100 allows dual or multi-region compliance for border regions or healthcare professionals operating across different regulatory zones. For example, if a clinician works in both Canada (Health Canada compliance) and the United States (FDA compliance), the system 100 may automatically adjust regulatory settings based on WiFi network recognition (e.g., detecting whether the device is connected to a hospital's network in either country).

In some examples, the system 100 continuously monitors regulatory database updates to adapt compliance settings, even in the absence of location changes. For example, if a new local regulatory framework is introduced, or if an existing one undergoes revisions (such as updated accuracy validation criteria under ISO 81060-2), the system 100 may dynamically integrate these changes to maintain continuous regulatory alignment. In some examples, the system initially auto-selects the appropriate regulatory framework based on location, but the user 110 retains the option to override this selection if needed. In some examples, if the device is deployed in a multinational clinical study, the system 100 can switch regulatory frameworks automatically based on the study's location, ensuring that all data collected adheres to the specific regulatory requirements imposed by trial oversight bodies in each country. By integrating automatic regulatory selection with real-time validation of the sensor and signal against pre-approved (or predetermined) criteria, the system 100 not only complies with medical device regulations dynamically, but also maintains the highest standards of clinical accuracy and legal compliance across global territories.

In some examples, the validation engine 134 applies the predetermined validation criteria to ensure that only pre-approved sensors are used for non-invasive blood pressure measurement. In some examples, the system 100 actively enforces sensor validation by cross-referencing the device or sensor in use (e.g., user device 102a, 102b or sensor 104, 114) with a pre-established list of qualified sensors (or devices). In some examples, the qualified sensors (or devices) have undergone clinical or bench-testing validation in the applicable regulatory territory. This approach ensures that each sensor integrated with the system 100 meets predefined accuracy and reliability standards, thereby reducing measurement errors, ensuring regulatory adherence, and maintaining device certification over time.

In some examples, the system 100 maintains a secure, dynamic database of qualified sensors (e.g., in the signal data database 136c) listing those that have been validated (e.g., through clinical studies or rigorous bench-testing under the applicable medical device regulations in different territories). In some examples, the sensor qualification process follows structured validation methodologies such as ISO 81060-2, IEEE 1708, or AAMI SP10, which define the accuracy and performance thresholds that non-invasive BP measurement devices must meet. Before allowing any sensor or device to be used for BP estimation, the validation engine 134 may check its hardware model, manufacturer ID, firmware version, and certification status against the database to confirm whether it has been explicitly approved for use in the given regulatory region.

In some examples, to qualify a sensor or device for inclusion in the list of qualified (or compatible) sensors/devices, clinical validation studies may be conducted to demonstrate that the sensor or device meets the accuracy and performance requirements required for medical certification. Such studies may include a multi-cohort clinical trial where the sensor's PPG signal quality, BP estimation accuracy, and reliability across diverse patient populations are assessed against a reference standard, such as double-auscultatory BP measurement by trained clinicians. The results of such studies may be used to define performance thresholds that must be met for a sensor/device to be deemed suitable for real-world deployment. Similarly, bench-testing methodologies may be used to evaluate sensor signal stability, optical properties, and environmental robustness, ensuring that light intensity, SNR, and motion artifact resistance fall within the predefined acceptable ranges. In some examples, the system 100 supports ongoing qualification updates, allowing manufacturers to submit new sensors/devices for certification and inclusion in the database. This process may involve an automated verification framework, where new sensors/devices undergo real-time compliance testing against predefined signal quality and performance benchmarks before being added to the list of validated sensors/devices. If a newly detected sensor/device is not in the approved list, the system 100 may either reject it outright or require manual verification by an administrator or regulatory body before authorizing its use.

In some examples, for further security and post-market compliance monitoring, the system 100 may periodically revalidate sensors/devices already on the compatibility list by tracking firmware updates, manufacturer modifications, or new regulatory changes that could impact sensor/device performance. If a previously validated sensor/device undergoes a hardware revision or software update, the system 100 may temporarily disable its use until requalification testing confirms that the modifications have not adversely affected measurement integrity. This approach mitigates risks associated with device drift, manufacturing inconsistencies, or unapproved alterations that could compromise regulatory compliance.

In some examples, territory-specific sensor/device validation ensures that only sensors/devices certified under the applicable local regulatory framework are used within a given region. For example, a sensor/device model approved under FDA 510(k) clearance in the United States may not automatically qualify for use under EU MDR requirements, which demand independent clinical validation under ISO 81060-2. The system 100 may dynamically enforce this restriction by checking the regulatory approval status of the sensor/device before permitting use. If the user 110 travels between regions where different regulatory requirements apply, the system 100 may (i) automatically adjust the regulatory framework and apply region-specific validation rules or (ii) prompt the user to confirm compliance status before continuing measurements. In a multi-sensor ecosystem where users can connect different third-party sensors/devices to the system 100, the qualification step may ensure interoperability without compromising regulatory compliance. If a user attempts to pair an unverified sensor/device, the system 100 may suggest an alternative validated sensor/device from the compatibility list, or recommend recalibration procedures if partial validation is available. This may be useful in environments where multiple sensor modalities (e.g., wrist-worn wearables, smartphones, or medical-grade pulse oximeters) are available for PPG-based blood pressure estimation. For device manufacturers and healthcare institutions, the validation framework ensures that approved sensors/devices consistently meet clinical accuracy requirements, preventing substandard or uncertified sensors/devices from being used in medical or telehealth applications. This feature is particularly useful for remote patient monitoring (RPM) systems, where regulatory compliance must be strictly enforced to ensure that clinicians receive high-fidelity BP data for medical decision-making.

The validation step ensures only qualified sensors/devices are authorized for use in non-invasive BP estimation. By dynamically cross-referencing real-time sensor data with an approved database, the system 100 proactively enforces regulatory compliance, guarantees the accuracy and reliability of every BP measurement, and mitigates risks associated with sensor degradation, unverified third-party integrations, or region-specific regulatory constraints.

In some examples, the validation engine 134 is configured to assess the signals produced by the sensors, ensuring continuous alignment with strict, regulatory-approved benchmarks. This validation process ensures that each individual BP measurement not only originates from a qualified sensor/device, but that the signals generated in real-world conditions comply with precise and stringent criteria, previously approved by regulatory authorities. To achieve this objective, the system 100 maintains a comprehensive library of validated sensor characteristics and corresponding signal quality benchmarks (e.g., in the signal data database 136c). In some examples, such signal data is initially derived through extensive clinical trials, bench-testing studies, and rigorous regulatory approval processes. For example, clinical studies conducted according to ISO 81060-2, IEEE 1708, or AAMI SP10 standards may have demonstrated that specific sensor attributes, such as optical wavelength (ranging typically from 530 nm green wavelengths up to 950 nm near-infrared), ensure reliable and accurate blood pressure estimates. Bench-testing further validates sensor hardware parameters, including source-detector separation distances, LED emission consistency, light intensity, pulse duration, modulation frequencies, analog filtering parameters, and sampling frequencies, confirming that each of these attributes significantly impacts the signal's accuracy and clinical utility. Sensors previously validated are thus documented with their respective operational characteristics, performance benchmarks, and regulatory-approved configurations in a centralized and secure reference database.

When introducing a new or previously unqualified sensor/device to the system 100, an automated signal validation process may compare the sensor-generated signals against the established benchmarks stored in the database. In some examples, The signal validation process comprehensively examines numerous sensor characteristics critical for achieving regulatory compliance and clinical accuracy. These parameters may include optical wavelength (typically within the visible, near-infrared, or infrared spectrum), source-detector separation distance, sampling frequency and analog-to-digital conversion rate, amplifier gain settings, analog filter specifications, pulse duration, and emitted optical power. Furthermore, the system 100 may evaluate specific temporal and spectral characteristics of the signals, including waveform morphology, inter-cycle consistency, SNR ratios, pulse amplitude variations, baseline drift levels, artifact occurrence rates, harmonic distortion metrics, and overall signal stability. IN some examples, the signal validation process incorporates environmental and contextual parameters known to influence sensor accuracy. Such parameters may encompass temperature compensation methods embedded within the sensor hardware, sensor response stability across diverse ambient temperature ranges, and sensitivity adjustments accounting for changes in user body temperature. By comparing real-time measured parameters against previously documented, clinically-validated sensor benchmarks, the system 100 can accurately predict whether the new sensor's outputs will reliably meet established regulatory requirements.

In some examples, the system 100 can evaluate the compatibility of the sensor's signal quality parameters based on the user's current physiological state (e.g., sitting, standing, lying down, or exercising), the environmental conditions (e.g., ambient temperature, humidity), and even regulatory-specific constraints dictated by the geographic territory where the sensor/device is utilized. In some examples, a holistic evaluation ensures that the sensor does not merely match previously validated hardware characteristics, but consistently produces signals meeting regulatory accuracy standards under diverse operational conditions. Additionally, this comprehensive validation mechanism aligns strategically with regulatory compliance processes. Regulatory bodies, including the FDA, European Union's notified bodies under EU MDR, Australia's Therapeutic Goods Administration (TGA), and Japan's PMDA, mandate rigorous clinical accuracy requirements for non-invasive BP devices. By demonstrating upfront that specific sensor parameters reliably produce regulatory-compliant outcomes, the system 100 can leverage regulatory approval for entire classes of sensors sharing those validated characteristics. Subsequently, the real-time validation step ensures ongoing regulatory compliance, automatically preventing the usage of sensors that drift out of approved operational tolerances or fail to match the established sensor benchmarks. In scenarios where sensors undergo manufacturing revisions, software updates, or configuration changes, the system's dynamic validation step continuously monitors and detects deviations, prompting revalidation or certification updates if necessary. In some examples, the predictive validation strategy dramatically streamlines the introduction of new sensor technologies into regulatory territories. Regulatory authorities may grant incremental approvals for sensor configurations closely matching previously validated benchmarks, substantially reducing both time-to-market and associated regulatory burdens. By ensuring that each individual BP measurement is continuously validated against established criteria, the system 100 not only mitigates regulatory risks but also guarantees sustained accuracy and reliability, promoting enhanced clinical confidence among healthcare providers, patients, and regulatory stakeholders.

The validation step (or process) ensures that the BP estimation model of the model engine 126 operates only on qualified inputs, thereby improving accuracy and robustness of the system 100. In some examples, the validation process assess the sensor 104 or 114 itself (e.g., its specifications, operational status, or compliance with predefined performance standards) and/or the received signal (e.g., verifying its integrity, stability, or physiological plausibility). As such, the validation step prevents unreliable or low-quality data-such as signals corrupted by motion artifacts, sensor misplacement, environmental interference, or physiological inconsistencies-from being processed by the BP estimation model, thus increasing the reliability of the BP estimation. By incorporating intrinsic signal validation with adaptive contextual validation, the system 100 ensures that BP measurements remain clinically relevant, physiologically appropriate, and regulatory-compliant across diverse conditions. These adaptive signal validation techniques enhance the reliability of non-invasive BP estimation, making it robust for both consumer health applications and medical-grade monitoring in real-world environments.

In some examples, the validation engine 134 is configured to perform a comprehensive multi-dimensional validation process encompassing signal integrity assessments, contextual information analysis, and regulatory conformity checks. Each component contributes distinctively, collectively enabling superior reliability, accuracy, and clinical trustworthiness of BP outputs. Signal integrity evaluation may include sophisticated signal processing techniques that rigorously assess fundamental physiological signal characteristics. For instance, the SNR can be evaluated through algorithms designed to separate the pulsatile signal component from background noise, ensuring that signals with insufficient SNR-potentially compromised by inadequate sensor contact or poor optical illumination-are flagged and excluded from clinical use. Furthermore, waveform stability assessments can employ methods such as inter-cycle variability analysis, where consecutive pulse waveforms are compared for consistency in amplitude, duration, and morphology, thereby identifying and discarding erratic or physiologically implausible waveforms. Artifact detection techniques further refine signal integrity validation by incorporating algorithms capable of recognizing motion-induced distortions, skin-contact inconsistencies, baseline drift, ambient light interference, or sudden signal spikes, each of which can degrade accuracy if not identified and excluded. Additionally, the validation engine 134 can verify physiological plausibility by ensuring that derived physiological parameters such as heart rate, pulse interval, and waveform morphology remain within expected human physiological ranges, thereby reinforcing the trustworthiness and clinical validity of each measurement.

A contextual analysis of the user's physiological state may include integrating information from supplementary embedded sensors such as accelerometers, gyroscopes, and heart rate monitors within wearable devices, as well as user inputs or external data sources. For example, user posture detection through embedded inertial sensors can identify whether the user 110 is standing, sitting, lying down, or transitioning between these states. Measurements performed while the user 110 is standing or moving can trigger additional validation criteria or be excluded to prevent posture-induced inaccuracies. Likewise, data regarding motion states, obtained from embedded accelerometers, can allow the system 100 to automatically detect periods of physical activity, differentiating between resting and active states. Signals captured during significant physical activity might be automatically marked for exclusion, or alternatively, subjected to advanced motion-compensated algorithms capable of preserving accuracy even under dynamic conditions. Similarly, sleep condition data obtained from integrated sleep tracking sensors, such as body temperature sensors, ambient light sensors, or external sleep trackers, can help refine measurements conducted during nighttime rest periods. Physiological signals recorded during sleep conditions, when cardiovascular states differ significantly from wakeful periods, can be validated against sleep-specific regulatory or physiological thresholds to ensure clinical relevance. Physical activity level inputs, such as those from external fitness tracking applications or user-reported exercise data, further allow the system 100 to dynamically adjust validation criteria, ensuring that transient, exercise-induced physiological changes do not skew clinical accuracy.

In some examples, the validation step links signal quality validation with regulatory compliance checks tailored to the specific medical device standards required in the geographic territory of use. Embodiments may utilize automatic geolocation or user-selected regulatory frameworks, thereby dynamically enforcing local regulatory compliance criteria at every measurement. For instance, signals recorded by sensors/devices used within European territories could undergo automatic real-time validation against accuracy criteria defined by ISO 81060-2, ensuring each measurement fulfills strict European CE marking criteria for BP monitoring devices. In contrast, devices utilized within the United States would automatically validate signal conformity against FDA-cleared requirements, referencing AAMI SP10 or IEEE 1708 guidelines. Thus, the system 100 may dynamically incorporate territory-specific signal quality standards, adjusting signal validation thresholds to meet mandated local accuracy and reliability requirements. If a signal fails to meet the applicable territory's regulatory criteria, the system can automatically prevent its clinical use, prompting remeasurement or notifying users that regulatory-approved accuracy was not achieved.

In one example configuration, a wrist-worn wearable employs an integrated accelerometer to detect user posture in real-time, dynamically adjusting the required SNR and waveform stability thresholds for signals obtained in sitting versus standing positions. Signals captured during movement may undergo motion-artifact compensation techniques or be flagged if they exceed allowable artifact thresholds, ensuring that physical activity does not introduce inaccuracies into clinical measurements. In a second example configuration, a smartphone-based sensor application may incorporate user-provided context information, allowing individuals to indicate whether they are relaxed or physically active prior to measurement, thus adapting validation criteria accordingly. The same system might utilize real-time geolocation data, automatically enforcing FDA-mandated accuracy thresholds within the U.S. and ISO 81060-2 criteria within Europe, ensuring seamless international compliance. In a third example configuration, an ear-worn device is capable of detecting sleep or waking states using integrated sleep-tracking sensors or external sleep monitoring data, dynamically adjusting validation criteria based on circadian physiology. In a fourth example configuration, a chest-worn patch is equipped with multiple sensor modalities (e.g., ECG, accelerometer, and optical sensors) and may simultaneously evaluate physiological plausibility and contextual factors, ensuring that BP estimates remain accurate across varied daily activities.

Regulatory-compliant algorithms, embedded directly within the wearable device or a connected smartphone application (e.g., client application 112), may continuously cross-reference acquired signals with pre-established territory-specific validation libraries, ensuring immediate compliance with medical regulations wherever the device is utilized. This integrated validation approach ensures that every non-invasive BP reading delivered by the system 100 meets stringent accuracy, reliability, and regulatory requirements under diverse real-world scenarios, greatly enhancing clinical confidence, regulatory compliance, user trust, and overall commercial value of the system 100.

As described above, the system 100 may employ adaptive mechanisms to dynamically adjust validation criteria according to varying measurement conditions, recognizing that distinct clinical or consumer use scenarios inherently demand different levels of performance precision from the BP estimation system. For instance, in clinical or diagnostic settings-such as hospital-based patient monitoring or hypertension diagnosis-medical regulations typically require stringent performance benchmarks, such as lower measurement biases and higher precision thresholds (e.g., within 5 mmHg bias and 8 mmHg precision). Under such conditions, the predetermined validation criteria for SNR, waveform stability, physiological plausibility, and contextual data, such as user posture and motion state, are adjusted to achieve maximum measurement fidelity. Conversely, in consumer-grade settings or scenarios involving routine health monitoring, slightly relaxed precision standards may be permissible (e.g., within 10 mmHg precision), enabling broader usability across varied daily activities and physiological states. In some examples, validation thresholds are adapted dynamically, allowing measurements in motion or under mild physical exertion conditions, provided the signals remain physiologically plausible and contextually consistent with a higher tolerance for variability.

Further, the system 100 may automatically modify validation criteria based on explicitly defined measurement contexts. For example, a wearable device may apply stringent criteria for signals collected during user-indicated “resting states” to ensure clinical-grade accuracy, whereas signals gathered during periods of physical activity may be validated using relaxed criteria, accommodating transient physiological variability without compromising usability. Similarly, measurements taken during sleep conditions-where the user's cardiovascular state exhibits distinct patterns, such as lower heart rates or decreased peripheral perfusion-can employ specialized criteria adapted specifically for nocturnal monitoring, ensuring both accuracy and clinical relevance.

At step 906, in response to validating at least one of the sensor or the received signal, the validation engine 134 delivers the signal to the BP estimation model of the model engine 126. As described above, the BP estimation model is configured to provide one or more BP values based on the validated signal. In some examples, the BP estimation model is a machine-learning model trained on extensive datasets encompassing various users, conditions, and sensor types. The BP estimation model may leverage deep learning algorithms, such as convolutional neural networks or recurrent neural networks, to accurately predict BP from the validated signal.

At step 908, the output BP value (or values) is delivered to the user 110. This delivery may be implemented via graphical displays on wearable devices, smartphones, tablets, or dedicated health monitoring dashboards. The delivered information may comprise instantaneous BP measurements or historical trends, enabling users and healthcare professionals to track BP changes over time, adjust interventions, or guide clinical decisions. In some examples, the BP value is transmitted back to the user device 102a or 102b. For example, the application server 124 may transmit the BP value to the first user device 102a, and the presentation engine 117 may configure the BP value for presentation to the user 110 via the display 106. Likewise, the application server 124 may transmit the BP value to the second user device 102b, and the second user device 102a may transmit the BP value to the first user device 102a for presentation to the user 110. In some examples, the model engine 126 delivers the output BP value to a database (e.g., user data database 136b) for storage and subsequent retrieval. Given that the final BP value is delivered to the user 110, the method 900 may be suitable for consumer health applications, telemedicine, and medical-grade remote monitoring.

In a consumer-focused wellness configuration, BP readings may be displayed directly on the wearable's screen, stored in a companion mobile app, or transmitted to voice assistant platforms for spoken feedback. For example, a smartwatch may provide graphical trends showing BP variations throughout the day, accompanied by personalized insights on lifestyle factors influencing fluctuations. In some examples, AI-powered health assistants may be used where the system 100 integrates with smart speaker or chatbot interfaces that deliver contextualized BP feedback and recommendations. In some examples, such BP feedback or recommendations are presented in the form of AI-generated images or videos. In medical applications, the system 100 may deliver BP results in structured formats suitable for electronic health record (EHR) integration, ensuring that clinicians receive standardized outputs compatible with HL7 FHIR (Fast Healthcare Interoperability Resources) protocols. For hospital-grade wearables, BP readings may be transmitted in DICOM-compliant reports, allowing direct incorporation into hospital information systems (HIS) for automated patient monitoring. In some examples, patients may to receive automated alerts via SMS, email, or notifications, ensuring that critical BP deviations trigger real-time intervention by healthcare providers.

In some examples, BP results are delivered directly to the device housing the sensor. In such examples, the system 100 enables self-contained usability, ideal for minimalist product designs or offline applications. In some examples, a smartwatch with a built-in PPG sensor displays BP readings directly on its OLED screen, allowing the user 110 to review historical data, observe daily trends, and set personalized BP thresholds. In some examples, a smart ring integrates a haptic feedback system, where vibrations signal elevated BP levels, allowing users to react promptly without needing to check a screen. In medical applications, portable BP monitoring patches with embedded e-ink displays may provide clinically accurate, on-device readings, ideal for elderly users or remote patient monitoring programs where simplicity and low maintenance are paramount. Additionally, wellness-focused smartphone-based implementations-where users measure BP by placing their finger over the camera lens-could ensure that BP values are displayed immediately within the client application 112, reinforcing real-time feedback without requiring cloud access. To ensure regulatory compliance, medical versions of these systems may implement on-device data encryption and audit trail logging, preventing unauthorized modifications to BP data while ensuring that readings remain accessible to both users and healthcare professionals.

In some examples, voice-based BP feedback is provided. BP values may be read aloud using a voice assistant for accessibility. For example, users can ask, “What's my latest blood pressure reading?” and receive a spoken response. In some examples, notification-based BP alerts and warnings are issued (e.g., via the client application 112). For example, the system 100 may notify the user 110 when their BP reaches a critical threshold. In one example, a smartwatch pushes a notification if BP is abnormally high, prompting the user 110 to rest or take medication. In another example, a smartphone app sends an emergency alert if BP values suggest hypertensive crisis, with a prompt to call a doctor. In some examples, the system 100 provides integration with Electronic Health Records (EHRs) and medical systems. The system 100 may send BP values to an EHR for clinician review.

In some examples, the system 100 presents a trend of BP values to the user 110. Instead of only showing individual BP readings, the system 100 may provide trend analysis, allowing users to see how their BP evolves over time. This helps identify patterns, track treatment effectiveness, and detect hypertension risks. In some examples, a graphical trend representation is presented (e.g., using line graphs, bar charts, or scatter plots). A 7-day BP trend graph in an app may allow users to compare their daily averages. Likewise, monthly and yearly BP trends allow users can see long-term changes and treatment effects. In some examples, an AI-powered BP trend analysis is presented. In such examples, the system 100 analyzes patterns and provides automated insights about BP fluctuations (e.g., “Your BP is trending higher in the evenings-consider reducing salt intake at dinner”). Such insights may be presented as AI-generated text, voice, audio, video, infographics, or as any other desired format. In some examples, a BP trend comparison with lifestyle and activity data is provided. For example, BP trends may be correlated with sleep, physical activity, diet, and medication adherence. In another example, BP trends may be shown alongside step count and exercise intensity, allowing users to see how physical activity impacts BP. Similarly, a dashboard may link BP trends to medication intake, reminding users to stay on track with their prescriptions. In some examples, a predictive BP trend analysis for Hypertension risk detection is provided. The system 100 may predicts future BP trends based on past data and user behavior. For example, a predictive BP model my warn users if they are at risk of developing chronic hypertension. In one example, a wearable device suggests adjustments in lifestyle before BP spikes occur. In some examples, BP trends are shared with healthcare providers for remote monitoring. BP trends may be automatically transmitted to doctors or caregivers for telemedicine applications. One such example is a home-care BP monitoring program, where elderly patients' BP trends are remotely tracked by clinicians. Another example is a virtual hypertension management program, where doctors adjust medications or user's change their daily habits based on weekly BP trends. In some examples, trend-based BP alerts and personalized recommendations are issued by the system 100. For example, the client application 112 may notify users if BP is consistently high in the mornings, suggesting lifestyle modifications. In another example, a wearable may alert users to seek medical advice if BP trends show a rapid increase over several days. In some examples, the system 100 provides detailed analytics dashboards for users who want granular control over their BP data. For example, a fitness enthusiast viewing BP fluctuations during different workouts to optimize training intensity, or a medical researcher tracking BP trends in study participants using an interactive web platform. In some examples, the system 100 includes gamification elements to encourage users to monitor BP trends regularly. For example, users may earn points for maintaining healthy BP trends, incentivizing good health behaviors. Similarly, a habit-tracking feature may be provided where users complete daily BP checks and receive progress badges.

It should be appreciated that the method 900 extends to various non-invasive sensors (e.g., sensors 104, 114), ensuring broad applicability across wearable devices, smartphone-integrated systems, and dedicated medical monitoring devices. By performing the method 900, the system 100 functions without a required user calibration, meaning that no external BP reference measurements, periodic recalibrations, or manual inputs of BP values are used to estimate the user's BP value(s). This represents a significant departure from existing cuffless BP estimation techniques, which typically rely on calibration steps to adjust for inter-user variability, or to compensate for signal inconsistencies. By eliminating such reliance on calibration, the method 900 enhances the usability, scalability, and real-world deploy-ability of the system 100.

While the above description of the method 900 includes performing the validation process using the validation engine 134 of the application server, it should be appreciated that the method 900 may be performed via a processor-based architecture that allows for flexibility in implementation. For example, the validation, BP estimation, and output delivery processes may occur on the same device as the sensor (e.g., user device 102a, 102b) or be distributed across remote processing units (e.g., the application server 124). When performed locally on the user devices 102a, 102b, validation and model engines of user devices 102a, 102b (not shown) may be configured to perform the steps described above with respect to the validation engine 134 and the model engine 126.

In some examples, the system 100 validates the quality of the signals within the BP estimation model. In such examples, the BP estimation model itself incorporates embedded mechanisms to quantify signal quality as part of the estimation process. The BP estimation model applies internal validation techniques before using the signal for BP prediction. Since validation is part of the model, there is no need for external pre-processing, reducing system complexity. In some examples, the model can learn from its own decision-making process and refine how it evaluates signal quality based on past performance. In some examples, this configuration provides end-to-end optimization. Quality validation and BP estimation are performed in a single computational pipeline, reducing latency and improving efficiency. In some examples, the model can dynamically weigh lower-quality signals less heavily or discard them altogether without requiring external intervention. In some examples, the model can internally adjust to differences in sensor type, placement, or user conditions, ensuring that only the best-quality data is used for training and inference.

In some examples, the system 100 validates the quality of the signals outside of the BP estimation model (e.g., outside of the model engine 126). In such examples, signal validation is performed by an external system component before the data is passed to the BP estimation model for BP estimation. The external component may be integrated into different parts of the system 100, such as the user device 102a, 102b itself, a mobile application (e.g., client application 112), a cloud-based server (e.g., application server 134), or an offline data processing pipeline. In some examples, given that the signal quality assessment is decoupled from the model, independent improvements can be made to the signal validation and BP prediction. Instead of embedding all validation logic within the model, lower-quality signals can be filtered out beforehand, reducing unnecessary computations. In some examples, signal validation can be performed in different locations within the system architecture, enabling flexibility in real-time and offline applications. Depending on the deployment scenario, signal quality checks may be performed instantaneously (e.g., for immediate feedback) or in batches (e.g., for retrospective analysis). In some examples, external validation can be performed across multiple sensors, ensuring cross-validation between different data streams (e.g., a smartwatch and a smartphone). In some examples, signal validation outside the model allows for independent audits and regulatory traceability, ensuring compliance with medical standards.

Below are multiple examples demonstrating how signal validation can be performed outside of the BP estimation model. It should be appreciated that the following examples are not intended to be limiting and may be used as standalone techniques or techniques that are combined to produce a complete standard.

Example 1: Embedded in the Wearable Device (Real-Time Processing). The measurement device itself (e.g., smartwatch, smart ring) performs signal validation before transmitting the data. This configuration may reduce network bandwidth usage by only sending high-quality data for processing.

Example 2: Integrated in a Mobile Application. A dedicated app running on the user's smartphone (e.g., client application 112) processes the PPG signal before passing it to the cloud-based BP estimation model. In some examples, the app provides real-time user feedback, prompting the user to adjust sensor placement or remain still if quality is low.

Example 3: Pre-Processing on a Cloud-Based Server. The measurement device transmits raw signals to a cloud platform (e.g., application server 134), where advanced ML techniques are applied to validate signal quality before inference. This approach may be suitable for high-complexity validation algorithms that require more computational resources than the measurement device can provide.

Example 4: Validation in a Remote Health Monitoring System. Hospitals, clinics, or research institutions may receive real-time physiological data from measurement devices and validate it before using it for clinical decision-making. This ensures that medical professionals receive only reliable and actionable information.

Example 5: Validation in a Machine Learning Data Pipeline (Offline Processing). Signal validation may performed retrospectively on large datasets collected from multiple users. This approach may be useful for improving model training by ensuring that only high-quality data is used for refining BP estimation algorithms.

Example 6: Integrated in a Telemedicine Platform. A telemedicine provider's backend system may process and validate the signal before presenting it to doctors or patients in a dashboard. This ensures that clinicians only receive meaningful and clinically relevant data.

Example 7: Edge Computing on a Companion Device (e.g., Smart Home Hub, Fitness Tracker Base Station). A home-based hub (e.g., a smart speaker, fitness tracker dock) processes raw PPG signals locally before sending refined data to the cloud. This reduces latency and enhances privacy by keeping sensitive health data localized.

Example 8: Validation in an AI-Assisted Health Assistant. A digital health assistant, such as a virtual coach powered by AI, may process and evaluate the PPG signal before advising the user on lifestyle modifications, medication adherence, or measurement conditions.

Each of these embodiments demonstrates how external signal validation can be implemented in various configurations, ensuring flexibility, adaptability, and clinical robustness in real-world use cases. The ability to choose between internal model validation and external system validation allows for optimal system design depending on performance, regulatory, and infrastructure constraints.

### Device Qualification

A device qualification process is used to validate and qualify the compatibility of user devices and sensors with a BP estimation model (e.g., the model of model engine 126). In some examples, the qualification process is a robust, structured qualification and validation process specifically designed to ensure that measurement devices (e.g., user devices 102a, 102b) used for non-invasive, calibration-free BP estimation comply with stringent regulatory and clinical performance standards. In some examples, the qualification process involves an analysis of the signal shape received from measurement devices, a determination and storage of specific configuration settings to ensure the signal conforms to predefined characteristics, and a subsequent verification and configuration of the measurement device as a certified, verified system. The device qualification process may be performed with respect to third-party devices to ensure their hardware meets predefined signal and performance criteria. In such examples, the device qualification process is a regulatory and technical gatekeeping function that includes signal integrity validation, regulatory compliance checks, and configuration standardization, ensuring that only devices meeting specific performance thresholds can leverage BP estimation models (and associated algorithms). In some examples, the device qualification process is used to determine the compatibility of a device for use with a calibration-free BP estimation model.

In some examples, the application server 124 is configured to implement a qualification framework that includes a Software Development Kit (SDK) and an Application Programming Interface (API) service. The qualification framework may enable seamless integration of the BP estimation model into third-party device ecosystems. By offering a standardized SDK, third-party manufacturers can easily implement validated, high-accuracy BP monitoring without the need to develop their own regulatory-compliant models. In some examples, the API service allows the application server 124 to offer BP monitoring as a plug-and-play feature to third-party devices (and sensors).

As described above, the system 100 is configured to estimate BP values through a structured qualification and verification process, ensuring accurate, consistent, and regulatory-compliant measurements from various devices. Initially, the system 100 communicates with a computational calculation model (e.g., the BP estimation model), responsible for deriving BP values from physiological signals received from measurement devices. In some examples, the qualification engine 132 of the application server 124 is configured to determine if the measurement device providing the signal is a “verified device.” In some examples, verified devices are defined as those which have successfully completed a qualification procedure.

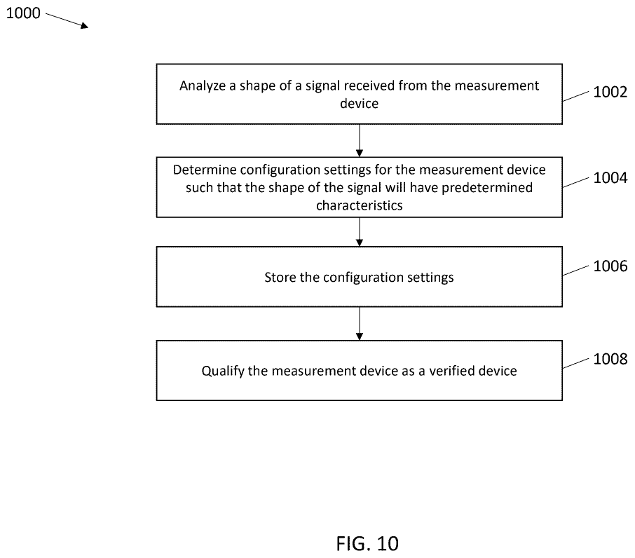

FIG. 10 illustrates a method 1000 for qualifying a user device for BP determination in accordance with aspects described herein. In some examples, the application server 124 of the system 100 is configured to perform the method 1000. In some examples, the method 1000 corresponds to the qualification procedure that is performed, at least in part, by the qualification engine 132 of the application server 124.

At step 1002, the qualification engine 132 analyzes a shape of a signal received from the measurement device. The signal may be a pulsatility signal, a physiological signal, or any other type of signal that is representative of data collected by a measurement device sensor. In some examples, the signal is pre-processed by the processing engine 130 of the application server 124 before being received by the qualification engine 132. In some examples, the measurement device is a third-party device (e.g., a wearable device, smartphone, medical device, etc.) or one of the user devices 102a, 102b.

In some examples, the qualification engine 132 verifies that the physiological signal waveform meets predetermined technical characteristics known to be critical for reliable BP estimation. In some examples, the qualification engine 132 is configured to analyze various morphological and statistical characteristics of the signal to determine whether a measurement device is suitable for delivering data to the calculation model. Such an analysis encompasses several distinct metrics, each providing critical insights into the integrity and physiological validity of the recorded signal. One example metric is amplitude analysis, where the amplitude of recorded signals is assessed to confirm they fall within an acceptable physiological range. This ensures that the measurement device consistently captures adequate pulsatile information, neither too weak-indicating poor optical coupling or insufficient sensitivity-nor excessively strong-implying optical saturation or incorrect analog front-end (AFE) settings. In some examples, the predetermined acceptance criteria specifies minimal and maximal acceptable amplitudes based on benchmarks derived from previous clinical validation trials.

Another example morphological parameter involves peak-to-peak analysis, where the qualification engine 132 evaluates the amplitude differences between consecutive peaks and troughs within the pulsatile waveform. By ensuring that these peak-to-peak variations remain within predetermined physiological boundaries, the qualification engine 132 guarantees that the measurement device accurately detects genuine arterial pulsations, free from artifacts or environmental interferences. Slope analysis further refines this evaluation by quantifying the steepness of waveform transitions, such as the ascending systolic slope or descending diastolic slope, verifying that recorded slopes align with typical physiological ranges. Devices exhibiting unusually shallow or excessively steep slopes may fail qualification criteria due to potential signal distortions or insufficient temporal resolution.

In some examples, an analysis of the DC component—or baseline offset—of the signal is performed to confirm that the optical sensor maintains stable contact with skin and optimal illumination conditions throughout data acquisition. A consistent and stable DC baseline may correspond to a reliable extraction of the pulsatile component, while significant baseline drift or fluctuations could indicate sensor instability, motion artifacts, or external interference.

Acceptance criteria may stipulate allowable DC drift tolerances over defined recording intervals to ensure measurement reliability.

In some examples, the qualification engine 132 evaluates waveform distortion metrics to quantify the degree of deviation from idealized reference waveforms obtained from clinical trials and/or previously approved sensors. Such distortion metrics may include harmonic distortion ratios, waveform asymmetry indices, or deviations from canonical PPG shapes derived from standardized databases. Meeting specific thresholds for these distortion measures ensures that the sensor configuration and electronic front-end accurately reproduce clinically relevant signals, preserving waveform integrity critical for precise blood pressure estimation.

The presence or absence of key physiological features within the recorded waveform constitutes another evaluation parameter considered by the qualification engine 132. The qualification engine 132 may evaluate the signal to confirm that certain physiological landmarks, such as clear systolic peaks, identifiable dicrotic notches, and stable diastolic troughs, consistently appear in the waveform. Predefined acceptance criteria specify the required frequency, visibility, and consistency of these features, with failing devices potentially indicating inadequate sensor placement, insufficient sensitivity, or excessive artifact contamination.

Further, the qualification procedure analyzes the relational metrics between physiological features, such as peak-to-notch intervals, systolic-diastolic amplitude ratios, and inter-beat intervals. These inter-feature relationships provide valuable contextual information confirming that the waveform morphology aligns with known physiological norms. Devices passing these relational metrics demonstrate reliable physiological fidelity essential for clinically valid blood pressure measurements.

In some examples, quality indices generated directly by the BP estimation model itself, such as confidence scores, model-derived signal quality indicators, and/or error metrics, are also integrated into the qualification framework. These indices may be used to assess the usability and clinical relevance of signals by correlating the measured data against trained reference models and signals. Additionally, correlation statistics computed between recorded signals and original reference signals obtained during clinical validations or simulations may serve as comprehensive benchmarks. High correlation coefficients exceeding predetermined thresholds indicate strong similarity and reliability, qualifying the device for integration with the calculation model.

In some examples, the qualification engine 132 is configured to evaluate signals using the BP estimation model of the model engine 126, providing an end-to-end validation solution. In such examples, signals recorded from a candidate measurement device—potentially via a simulator reproducing real subject waveforms from pivotal clinical trials—are processed by the BP estimation model, producing estimated BP values. These estimated BP values are then statistically compared to known reference BP values associated with the underlying reproduced signals. Such reference values may derive from the original pivotal clinical trial dataset or from carefully curated reference measurements captured using validated clinical methods. In some examples, the primary acceptance criteria for qualification in this embodiment includes statistical metrics quantifying differences between estimated and reference BP values. Metrics may include mean absolute difference (MAD), mean squared error (MSE), bias (average difference), standard deviation of differences (precision), correlation coefficients between model-generated and reference BP, or confidence intervals of such metrics. For example, predefined acceptance thresholds may mandate an average bias below ±5 mmHg, precision within ±8 mmHg, and correlation coefficients exceeding 0.85. Devices consistently achieving these predefined criteria across all simulated clinical conditions would be considered qualified for delivering data to the BP estimation model, ensuring clinical accuracy and regulatory compliance. Moreover, the acceptance criteria can be applied at different granularities, such as on a measurement-by-measurement basis or aggregated at the subject level. At the measurement level, the qualification engine 132 computes statistics independently for each simulated measurement scenario, verifying whether individual signal segments reliably yield BP estimates within clinically acceptable boundaries. This validation approach ensures device robustness even under transient physiological conditions or measurement variability. Alternatively, subject-level aggregation provides broader performance evaluation by averaging multiple measurements recorded across various simulated physiological states or time points for each subject. This approach ensures device accuracy over sustained monitoring periods, reflecting real-world usage scenarios. Combining both measurement-level and subject-level statistics provides comprehensive qualification, ensuring candidate devices meet clinical accuracy standards across diverse conditions. This dual approach effectively demonstrates that the qualified device is capable of consistently providing clinically validated BP readings—directly confirming the real-world clinical performance anticipated in regulatory submissions.

In addition to comparing calculated BP values from candidate devices against rigorous clinical-grade reference devices, the qualification process may leverage reference BP values from a variety of alternative, more accessible, yet potentially noisier sources. One example involves using widely available commercial-grade BP monitors, such as automated oscillometric cuffs purchased by end users for home monitoring. While commercial oscillometric devices typically exhibit increased variability compared to clinical-grade manual auscultatory or invasive monitors, their widespread availability makes them particularly attractive as reference devices for large-scale qualification campaigns. In this scenario, the candidate measurement device captures pulsatility signals simultaneously with a commercial BP monitor reading. The BP estimation model may process the captured signals to estimate BP, and the statistical comparison between model-estimated BP and commercial monitor readings is subsequently performed. Although the noisier nature of commercial references necessitates more flexible acceptance criteria-for instance, allowing slightly higher acceptable bias thresholds (e.g., ±8 mmHg) and precision tolerances (e.g., ±10-12 mmHg)—the advantage of significantly enhanced scalability and lower operational costs makes this approach viable for rapid and expansive device qualification.

In some examples, the qualification engine 132 utilizes reference BP values derived from large-scale data collection campaigns, which inherently involve noisier datasets. Such campaigns may include remote or decentralized data acquisitions, crowdsourced via consumer-grade devices, smartphone apps, or wearable platforms deployed among thousands or even millions of users. This approach vastly expands the diversity and representativeness of reference data, capturing extensive real-world variability in user conditions, demographic profiles, and physiological states. In this context, the candidate measurement device's signals are again processed by the BP estimation model, with resultant estimated BP values statistically benchmarked against aggregate data collected during the large-scale campaign. Given the heterogeneity and inherent noise levels, statistical methods used for qualification may shift towards robust statistical frameworks designed to accommodate higher variance, such as median absolute deviation, robust regression, or percentile-based accuracy criteria (e.g., 90% of measurements within ±10-15 mmHg of the reference). Despite the relaxed tolerances required by noisier large-scale datasets, this approach allows continuous qualification of new devices in highly realistic and heterogeneous populations, greatly increasing generalizability and practical validity of the qualification.

In some examples, the qualification engine 132 utilizes previously qualified measurement devices as reference standards for qualifying subsequent new devices, thus enabling a sequential, iterative, and scalable qualification framework. Once an initial device—such as a specific smartphone model or a wearable optical sensor—has been rigorously qualified against clinical-grade references, it can itself serve as a reference standard (i.e., “source-of-truth”) for subsequent qualification processes. This iterative method allows progressive expansion of a device qualification library, significantly accelerating qualification rates. For instance, if one particular smartphone model (Model A) has already been qualified against clinical-grade auscultatory references with stringent statistical acceptance criteria (e.g., ±5 mmHg bias, ±8 mmHg precision, and correlation >0.85), the signals or estimated BP values obtained from Model A may be leveraged as reference data for evaluating new smartphone models (Model B, Model C, etc.). Thus, qualification tests for subsequent smartphone models can involve direct signal-to-signal or estimated BP-to-BP comparisons between the candidate device and the already qualified reference device.

In some examples, the iterative qualification approach may be carried out both in controlled laboratory settings and at larger scales through real-world beta-testing programs or large-scale clinical trial frameworks. In laboratory scenarios, standardized benchtop setups simultaneously expose candidate and reference devices to identical pulsatility signals generated by sophisticated optical signal simulators or pulsatile phantoms. This controlled environment ensures precise and reproducible test conditions, enabling strict signal-domain comparisons based on metrics such as waveform correlation coefficients, normalized cross-correlation indexes, frequency-domain spectral coherence, signal distortion metrics, or shape similarity scores. For BP-domain comparisons, statistical metrics such as mean absolute difference (MAD), bias, precision (standard deviation of differences), Bland-Altman analysis limits of agreement, or correlation coefficients could be systematically calculated. Acceptance criteria in lab conditions may maintain stringent thresholds to ensure the candidate device's performance is practically indistinguishable from the qualified reference device.

In large-scale real-world implementations, qualification can leverage beta-testing cohorts, crowdsourced user participation, or structured clinical trials involving hundreds or thousands of users simultaneously using both candidate and qualified reference devices. Beta-testers equipped with an already qualified device (e.g., such as smartphone Model A) and a candidate device (e.g., Model B) can collect simultaneous or temporally close pulsatility signals or estimated BP values during normal daily activities. Cloud-based infrastructures facilitate real-time or batch-mode statistical analyses, continuously comparing the candidate devices' performance against the previously qualified devices. Due to increased variability inherent in real-world scenarios, statistical acceptance criteria in these settings might relax slightly—for example, a permissible bias of ±8 mmHg, precision within ±10 mmHg, and correlation coefficients of at least 0.80—while still ensuring practical clinical accuracy and compliance with regulatory standards.

Moreover, performing these qualifications within large-scale clinical trial frameworks adds another layer of validation rigor and regulatory credibility. Candidate and reference devices can be deployed in multi-center clinical trials across diverse geographic regions, demographic groups, and physiological profiles. Trial participants simultaneously or sequentially use both candidate and qualified devices under standardized conditions, thereby generating extensive paired datasets. Qualification metrics can include not only previously mentioned statistics but also advanced analytic approaches such as linear mixed-effects models, Deming regression analyses, equivalence testing, and clinically meaningful error classifications (e.g., grades of hypertension misclassification rates). The regulatory approval process for qualification outcomes in clinical trial contexts typically involves direct oversight by regulatory agencies (e.g., FDA, EMA), bolstering the credibility, acceptance, and commercial value of the device qualification process.

By adopting flexible reference sources—including clinical-grade monitors, commercial devices, large-scale data campaigns, and sequential device-to-device benchmarking—the qualification engine 132 can significantly enhance scalability, efficiency, and robustness of new measurement device evaluations. These diverse approaches to generating reference data, together with adaptive statistical metrics and acceptance criteria, ensure that device qualification processes align with practical real-world usage scenarios, regulatory requirements, and clinical needs. Ultimately, such a multifaceted qualification framework facilitates rapid and cost-effective introduction of clinically validated BP measurement technologies into the broader consumer and medical markets.

In some examples, once a new device (e.g., Model B or C) has been successfully qualified against the initially qualified reference device (e.g., Model A), this newly qualified device itself can subsequently serve as a secondary reference device for qualifying yet another candidate device (e.g., Model D). This approach leverages the already established trust in the performance of intermediate qualified devices to expand the qualification library progressively. However, due to the inherent noise and variability introduced by propagating the qualification reference further away from the original clinical-grade baseline, it may become practical to adapt acceptance thresholds slightly. For instance, if Model A initially qualified Model B with tight statistical acceptance thresholds (e.g., bias within ±5 mmHg, precision within ±8 mmHg), the qualification of Model D using Model B as reference might require modestly relaxed thresholds (e.g., bias within ±6-7 mmHg, precision within ±9-10 mmHg), reflecting the incremental uncertainty accumulated through successive comparisons. Despite these adapted thresholds, maintaining a structured protocol of periodic controls—such as limited laboratory testing of Model D with simulators or periodic benchmarking with a reduced cohort of subjects against a clinical-grade reference—helps mitigate risks associated with propagated errors and ensures sustained accuracy and regulatory compliance. This combination of iterative qualification, threshold adaptation, and targeted verification controls represents an innovative, scalable strategy enabling rapid yet robust expansion of qualified devices across the consumer and clinical landscape.

At step 1004, the qualification engine 132 determines configuration settings for the measurement device such that the shape of the signal will have predetermined characteristics. Such configuration settings may be used to optimize the measurement device's operation, ensuring the captured signals consistently meet desired technical specifications. In some examples, the configuration setting (or parameters) include adjustments in sensor illumination intensity, exposure durations, sampling frequency, or sensor-specific analog front-end settings.

In some examples, a plurality of configuration settings may be adjusted in the sensor or device to optimize the quality of the recorded pulsatility signal and ensure compatibility with the BP estimation model. On the optical side, key settings (or parameters) include the light intensity of the LED emitter, which determines how much optical power is transmitted into the skin. Higher intensity can improve penetration depth and enhance signal quality, but excessive brightness may lead to overheating or unnecessary power consumption. Light exposure time, which defines the duration that the LED remains active per sampling cycle, is another setting that influences signal clarity, particularly in low-perfusion conditions where prolonged illumination may improve signal-to-noise ratios. LED wavelength selection is crucial as different wavelengths penetrate the skin to varying depths. Green light (i.e., 530 nm) is commonly used for wrist-based PPG due to its optimal absorption by hemoglobin, while infrared (i.e., 850-950 nm) is often preferred for deeper tissue penetration, such as in ear-based or fingertip PPG systems. In some examples, multi-wavelength configurations may be deployed to extract additional physiological information and enhance accuracy across varying skin tones.

The modulation mode of the LED driving current affects how light is emitted. Continuous wave (CW) illumination maintains a steady-state light output, while pulsed-mode operation alternates between on and off states, reducing power consumption and allowing ambient light subtraction techniques. The pulse width modulation (PWM) frequency governs the rate at which the LED pulses, with higher frequencies minimizing interference from ambient light fluctuations. Inter-sample timing, or the interval between consecutive LED activations, impacts the effective temporal resolution of the PPG signal. A high sampling rate enhances signal fidelity but increases data storage and processing demands. Duty cycle, defined as the ratio of active LED time to the total sampling period, may be optimized to balance power efficiency and signal clarity.

On the analog front-end (AFE) side, photodetector gain control is a key setting that adjusts the amplification of the received optical signal. Adaptive gain control dynamically adjusts sensitivity based on ambient conditions, ensuring stable output across varying illumination levels. Photodetector integration time refers to the duration the sensor accumulates light before conversion to an electrical signal; longer integration improves sensitivity but may introduce motion artifacts. Noise filtering settings, such as low-pass and high-pass filters, help eliminate undesired frequency components, removing baseline drift while preserving relevant pulsatility features. Ambient light rejection mechanisms, including active noise cancellation techniques, compensate for interference from external light sources, preventing contamination of the PPG waveform.

The sampling rate of an analog-to-digital converter (ADC) determines how frequently the signal is digitized. A typical PPG sensor operates between 25-1000 Hz, with higher rates capturing finer details of the waveform while increasing processing demands. Bit depth of ADC conversion, often 12-bit or 16- bit, affects the resolution of the digitized signal. Baseline correction algorithms adjust for slow drift in the signal caused by temperature fluctuations or sensor repositioning. Dynamic range adjustments ensure that the captured signal remains within the operational limits of the sensor, avoiding signal saturation or clipping. Cross-talk compensation techniques prevent interference between multiple LEDs or sensor channels when multi-wavelength PPG is used.

On the data recording side, signal compression settings determine whether raw, minimally processed, or fully preprocessed data is stored. Lossless compression formats retain all information for retrospective analysis, while lossy compression methods reduce file size at the cost of minor information loss. Data format standardization enables compatibility with external processing systems, ensuring interoperability between different devices and platforms. Synchronization protocols are essential when PPG signals are combined with data from other biosensors, such as electrocardiogramaccelerometry, to enable multimodal physiological analysis.

For camera-based video acquisition, a different set of configuration settings govern the optical signal recording process. Camera resolution defines the number of pixels captured per frame, with higher resolutions providing finer detail but increasing storage and processing requirements. Frame rate is relevant in video-based PPG, where higher frame rates (e.g., 60-240 fps) allow for more precise pulsatility detection. Exposure time per frame controls how long the sensor gathers light, affecting brightness and motion blur. Short exposure times minimize motion artifacts, while longer exposures enhance signal sensitivity in low-light conditions. ISO sensitivity settings determine the camera sensor's responsiveness to light, with higher ISO values improving low-light performance but introducing additional noise. White balance adjustments optimize color accuracy, preventing incorrect signal processing due to variations in skin tone and ambient lighting.

Focus mode selection is relevant in video-based PPG acquisition, where fixed-focus settings ensure that the camera remains optimally aligned with the skin, avoiding fluctuations in image sharpness. Compression settings for video encoding impact the fidelity of the extracted signal. Lossless formats, such as RAW or uncompressed YUV, preserve the most detail, while lossy formats, such as H.264 or HEVC, introduce artifacts that may degrade signal quality. Bitrate control adjusts the amount of data allocated per second of video, balancing file size with image integrity. Color depth and chroma subsampling settings impact how finely color and brightness variations are captured, which is relevant when extracting pulsatility signals from skin tone fluctuations.

Video stabilization settings influence motion artifact reduction. Some smartphones implement electronic or optical image stabilization (EIS/OIS), which can be beneficial in reducing involuntary hand movements when the user places a fingertip over the camera. However, excessive stabilization may introduce unintended distortions in the pulsatility signal. HDR (High Dynamic Range) processing enhances contrast but may affect temporal signal consistency. Shutter speed adjustments fine-tune exposure duration and can be synchronized with LED flash activation for enhanced signal capture. Flash intensity control determines how much additional light is emitted to enhance skin illumination, while flash timing synchronization ensures optimal alignment between light pulses and camera capture frames.

On the software side, video preprocessing pipelines apply noise reduction, contrast enhancement, and skin tone correction algorithms before signal extraction. Edge detection and region-of-interest (ROI) tracking isolate the area of the video frame containing the user's fingertip, preventing background noise from contaminating the extracted signal. Multi-frame averaging techniques aggregate data over consecutive frames to improve signal stability, while frame dropout compensation algorithms handle instances where temporary occlusions or fluctuations disrupt signal continuity.

For smartphone-based implementations, real-time video signal processing methods can be optimized for energy efficiency, particularly for continuous monitoring applications. Adaptive processing pipelines dynamically adjust camera settings based on ambient lighting conditions, ensuring consistent signal quality across different usage scenarios. ML-based artifact detection can flag corrupted frames, removing them from analysis to improve accuracy. Cloud-based versus on-device processing strategies balance computational load, allowing resource-intensive signal extraction to be offloaded to cloud servers when necessary.

The combination of these configuration settings (or parameters) allows for robust adaptation of PPG acquisition across different device types and usage conditions. By systematically adjusting these settings, signal quality can be optimized for both commercial wellness applications and regulatory-compliant medical device implementations.

In some examples, the qualification engine 132 determines optimized an adjustment of configuration settings, including but not limited to light intensity, light exposure time, and other parameters relevant to the acquisition of optical signals for BP estimation. In the case where the measurement device includes a camera, the configuration settings that can be modified include a wide range of parameters exposed by the device's native operating system frameworks, such as AVFoundation for iOS and Camera2 for Android. These frameworks provide extensive controls that allow the qualification engine 132 to optimize video-based PPG acquisition by dynamically adjusting camera properties to ensure signal quality and regulatory compliance.

On iOS devices, the AVFoundation framework enables control over the camera's session presets, determining the resolution and quality of the video stream being captured. The frame rate may be adjusted using active video frame duration settings, optimizing the acquisition of pulsatility waveforms at a frequency compatible with physiological signals. Focus mode settings, such as continuous autofocus or locked focus, allows for adjustment to maintain a sharp and stable image of the illuminated skin region, reducing variability caused by small movements. Exposure mode settings can be dynamically managed to compensate for changes in ambient lighting, ensuring that the recorded video maintains the required contrast for accurate pulse waveform extraction. Similarly, white balance adjustments can be applied in real time to prevent color shifts that may affect the detection of pulsatile variations in blood volume. Additional controls such as video stabilization help reduce noise caused by involuntary hand tremors when users manually position their fingertips on the smartphone camera. The torch mode can also be activated to provide a controlled illumination source, improving signal acquisition in low-light conditions. Zoom factor settings allow the system to optimize the recording frame for a specific skin region, ensuring maximum signal clarity.

On Android devices, the Camera2 API provides similar low-level control over video acquisition. The optimal resolution may be selected based on the device's supported stream configurations, ensuring compatibility with the processing model's requirements. The frame rate can be dynamically controlled to align with the desired sampling frequency, reducing aliasing artifacts when extracting pulsatility waveforms. Autofocus modes can be set to continuous tracking, ensuring that the recorded region remains sharply defined, or switched to a fixed mode to minimize unnecessary refocusing. Exposure compensation parameters allow for adaption to varying environmental lighting conditions, preventing overexposure or underexposure that may degrade PPG signal quality. White balance settings can be tuned based on detected ambient light conditions to enhance the accuracy of the recorded waveform. Video stabilization may be enabled to counteract small movements that may introduce motion artifacts into the recorded signal. Flash control can be used to provide additional illumination where necessary, creating a stable and reproducible lighting environment for the measurement. Digital zoom adjustments optimize the captured field of view to focus on the most relevant skin region, minimizing interference from surrounding areas.

Beyond optical signal acquisition, the qualification engine 132 can configure additional parameters related to video encoding and storage. On both iOS and Android platforms, bitrate settings may be modified to optimize the trade-off between file size and signal fidelity. Higher bitrate settings can preserve fine details in the pulsatility waveform, while lower bitrates may introduce compression artifacts that degrade signal quality. Audio settings may also be controlled if necessary, particularly in applications where background noise or secondary acoustic signals might influence waveform interpretation. The qualification engine 132 can select different video encoding formats and compression schemes to ensure compatibility with downstream processing pipelines while maintaining the integrity of the acquired signals.

By dynamically adjusting these configuration settings, the qualification engine 132 ensures that optical signal acquisition remains optimized across a variety of device models, lighting conditions, and user interactions. This flexibility enhances the robustness of BP estimation across a diverse user base, enabling reliable measurements under real-world conditions. Additionally, because these configuration settings can be modified programmatically, the system 100 can adapt to new device capabilities as smartphone camera technology continues to evolve, ensuring long-term scalability and compatibility.

In cases where the measurement device comprises a dedicated PPG sensor rather than a smartphone camera, the qualification engine 132 provides equivalent configuration control over various sensor parameters. The light intensity of the PPG sensor may be dynamically adjusted to optimize penetration depth and SNR, compensating for factors such as skin tone, tissue density, and ambient light interference. Light exposure time can be configured to ensure adequate photon absorption by the photodetector, maximizing the contrast of the pulsatile waveform. The wavelength of emitted light may also be adjustable, allowing the qualification engine 132 to select the optimal spectral range for a given use case-such as green light (i.e., 530 nm) for wrist-worn sensors, red or near-infrared light for fingertip measurements, or multiple wavelengths for multi-channel sensing applications.

In some examples, the qualification engine 132 supports configuration settings related to the analog front end (AFE), which processes raw optical signals before digital conversion. Amplifier gain settings can be modified to adjust the sensitivity of the sensor, preventing signal saturation in high-perfusion conditions or enhancing weak signals in low-perfusion cases. Bandpass filtering can be applied to isolate relevant pulsatility components while suppressing unwanted noise. Sampling rate configurations ensure that the sensor acquires data at an appropriate frequency for accurate pulse wave analysis. Dynamic range settings may be tuned to accommodate different skin types and physiological conditions, reducing the likelihood of clipping or distortion.

Further configuration settings may apply to the data recording format. The signal resolution (i.e., bit depth) can be adjusted to balance data precision against storage requirements. The system 100 may implement adaptive compression techniques to optimize storage efficiency without compromising signal integrity. Data transmission protocols can be configured to ensure secure and efficient communication between the measurement device and the processing unit (e.g., the application server 124), particularly in scenarios where cloud-based analysis is performed.

By offering granular control over these configuration settings, the system 100 enhances the accuracy, reliability, and adaptability of its non-invasive BP estimation capabilities. The ability to adjust optical and electronic parameters ensures that the system 100 can maintain high performance across different device types, operating conditions, and user populations. Furthermore, because these settings can be dynamically modified, the system 100 remains adaptable to emerging sensor technologies, regulatory requirements, and clinical validation protocols, reinforcing its viability as a scalable and commercially deployable medical and wellness application.

At step 1006, the qualification engine 132 stores the configuration settings for the measurement device. In some examples, the configuration settings are stored in the measurement device's memory (e.g., memory 108, 118) or a local configuration library. In some examples, the configuration settings are stored in a database associated with the application server 134 (e.g., the signal data database 136c).

In some examples, once a measurement device type has successfully passed the qualification process, its optimized configuration settings are securely stored in a cloud repository (e.g., a repository of database 136c). Each time a user attempts to use the system 100 with a new device, the qualification engine 132 checks against this repository to retrieve the correct configuration settings and determine whether the device has been previously qualified. This approach allows real-time updates and ensures that all devices accessing the system adhere to the latest validated parameters.

In some examples, the configuration settings are stored locally within the measurement device itself (e.g. memory 108, 118). For example, a smartwatch or smartphone may receive a firmware update containing its specific configuration settings once it has been verified as a qualified device. This ensures that the device operates using pre-approved acquisition parameters without requiring a continuous connection to a cloud service. In some examples, the configuration settings can be embedded in the device's software application layer, such as within the client application 112 that facilitates PPG data acquisition and transmission to the backend BP estimation model. In such examples, the client application 112 itself contains the necessary parameters to ensure compliance with the qualification criteria, providing flexibility for offline operation.

In some examples, the configuration settings can be stored within a regulatory-verified software module (e.g., the model engine 126) or a secure enclave embedded in the measurement device's hardware or software infrastructure. This approach may be beneficial for medical-grade devices that must ensure compliance with specific regulatory frameworks, such as FDA or MDR requirements. By storing the configuration settings in a secured environment, the system 100 can prevent unauthorized modifications that could compromise measurement accuracy or regulatory compliance.

At step 1008, the qualification engine 132 qualifies the measurement device as a verified device.

In some examples, the qualification status can be stored within a regulatory-verified software module (e.g., the model engine 126) or a secure enclave embedded in the measurement device's hardware or software infrastructure. By storing the qualification status in a secured environment, the system 100 can prevent unauthorized modifications that could compromise measurement accuracy or regulatory compliance.

In some examples, a device is flagged as a verified measurement device through a distributed verification mechanism. Once a new measurement device type has been successfully qualified, its identification information (e.g., device model, manufacturer details, firmware version, sensor characteristics, etc.) is added to an authorized device list stored either in the cloud or on regulatory-compliant local storage. Each time a user attempts to connect a new device to the system 100, a verification check ensures that the device is on this authorized list. If the device is not recognized, it may be flagged for further validation or denied access to the blood pressure calculation model.

In some examples, the qualification status can be assigned at the user account level within a cloud-based health platform. For example, if a user attempts to connect a new device, the system 100 can check against their profile to determine whether that device type has been pre-approved or if additional qualification steps are necessary. This enables user-specific device management, allowing flexibility in personalized sensor calibration and regulatory compliance tracking.

In some examples, a third-party verification service, such as a regulatory body or an independent verification entity, maintains a list of qualified devices and associated configuration settings. The system 100 (e.g., the qualification engine 132) may query this external service whenever a new device is connected, ensuring that only authorized and certified measurement devices interact with BP estimation model. This is particularly useful for large-scale commercial deployment, where multiple manufacturers integrate with the system 100 and require standardized qualification processes.

In some examples, the qualification status of a device is revalidated periodically to account for hardware modifications, firmware updates, or changing regulatory standards. If a previously verified device undergoes a significant update, the system 100 may require a requalification process to ensure that it continues to meet the necessary performance standards. This ensures that ongoing system reliability is maintained, even as devices evolve over time.

As described above, the qualification process may be performed by the qualification engine 132. In such examples, the qualification process is executed by one or more processors of the application server 124. In some examples, the qualification process is performed locally on the measurement device (e.g., via client application 112). In such examples, the qualification process may be executed by one or more processors of the measurement device. While an automated, processor-driven qualification process provides seamless integration and scalability, in certain scenarios, it may be beneficial to perform the qualification process externally to ensure rigorous, high-precision validation, regulatory compliance, and adaptation to different technological and clinical constraints. In such cases, the qualification process may occur outside of the measurement device or the application server 124, or even outside of the system 100 entirely.

In some examples, the qualification process is performed externally in a controlled clinical trial environment before the measurement device is authorized to interact with the system 100. In this scenario, third-party device manufacturers or medical device companies may submit their devices for evaluation under controlled, standardized conditions in a clinical research setting. This process involves collecting large-scale, high-quality datasets of signals generated by the candidate device while simultaneously obtaining reference BP values from a reference methodology, such as, for example, a double-auscultatory sphygmomanometry performed by trained clinical staff. Once the candidate device has undergone rigorous testing and its signals have been demonstrated to meet the predetermined qualification criteria (e.g., waveform morphology consistency, SNR thresholds, and physiological plausibility), it can be formally added to the list of pre-qualified devices. This manual qualification process ensures that only thoroughly vetted devices are permitted to transmit signals to the system 100 for BP estimation.

In some examples, the qualification process is conducted in a laboratory environment rather than a live clinical setting. In this case, instead of relying on real-world human subjects to generate PPG signals, the system 100 employs physiological simulators or synthetic signal generators that can reproduce arterial pulsatility waveforms corresponding to different BP values. The candidate device is placed in a controlled optical testing setup, where its ability to capture accurate PPG waveforms is analyzed across multiple simulated conditions, such as, for example, varying skin tones, motion artifacts, ambient light interference, and temperature fluctuations. By using high-precision laboratory instruments, researchers can objectively determine whether the device's recorded signals adhere to the system's predetermined qualification criteria. If the device passes this benchtop qualification, it can be integrated into the system 100 as a verified device without requiring additional clinical trials.

In some examples, the qualification process is conducted using simulated data, real subject data, and/or a combination thereof, as opposed to conducting full clinical trials for every new device integration. This approach leverages predefined acceptance criteria established by the entity responsible for the calculation model, ensuring regulatory compliance while dramatically improving the efficiency, speed, and cost-effectiveness of the device qualification process.

In some examples, the qualification process involves testing candidate devices in a laboratory setting using simulated optical signals. In such examples, dedicated optical simulators may be used to generate synthetic pulsatile waveforms replicating realistic physiological signals across a wide range of conditions, encompassing diverse cardiovascular profiles such as normotensive, hypertensive, hypotensive, arrhythmic, and varying arterial stiffness. The simulators may employ programmable optical modulation techniques, accurately emulating the pulsatile intensity variations observed in real PPG waveforms. Devices under test are positioned on or near the simulator's optical output source, allowing engineers to evaluate sensor performance, sensitivity, optical resolution, SNR, and waveform fidelity. Because these tests do not require recruiting human subjects or undergoing regulatory approval processes, they dramatically reduce the time, costs, and logistical complexity typically associated with full-scale clinical trials, allowing rapid iteration, troubleshooting, and validation of hardware designs.

In some examples, optical simulators are used to reproduce optical signals originally recorded from real human subjects. In this approach, previously captured clinical signals-potentially obtained from an initial clinical trial-are played back through laboratory-based optical emission systems, effectively enabling repeated “virtual clinical trials” in a controlled environment. This method allows each new device undergoing qualification to virtually experience the same physiological variability and conditions as encountered during pivotal trials, ensuring that device performance matches regulatory-approved clinical benchmarks. Importantly, since these signals derive from real subjects and representative patient cohorts, this embodiment provides highly realistic validation scenarios, bridging the gap between purely synthetic data and actual clinical environments. Devices can thus demonstrate their ability to capture clinically relevant signals across diverse patient populations without incurring the costs or complexity of recruiting new participants.

In some examples, optical simulators are not only used to modulate a beam of light, but also to incorporate physical phantoms designed to emulate the complex optical properties of human tissue. Such phantoms can include layers of diffusive materials, absorbing media, and pulsating mechanical structures that simulate arterial pulsatility within the phantom's volume. By placing candidate devices in direct contact with these phantoms, the qualification process tests more than merely sensor resolution or electronic front-end characteristics; it rigorously assesses the real-world ergonomics, optical coupling efficiency, and photon collection capability from complex biological-like structures. This embodiment represents a significant advancement beyond simple optical modulation, providing realistic feedback on sensor placement, mechanical stability, and how effectively the device captures pulsatile signals transmitted through biological media. It ensures that validated devices will reliably perform in actual physiological settings, substantially increasing confidence in clinical accuracy and regulatory compliance.

In some examples, the qualification process is performed semi-automatically outside of the system 100, using hybrid validation methodologies that involve both laboratory testing and real-world clinical performance assessments. For example, a new sensor technology may first undergo a series of standardized bench tests to confirm its optical, electronic, and signal-processing characteristics, ensuring alignment with previously validated devices. Following successful benchtop evaluation, a post-market surveillance or real-world data collection study may be conducted, where the device is deployed to a group of real-world users to validate its performance under natural usage conditions. This combined approach leverages the efficiency of benchtop testing while ensuring real-world robustness before fully certifying a device for large-scale deployment.

In some examples, the qualification process extends into carefully controlled, limited-scope human subject studies. These in-vivo qualification tests may involve selecting a representative set of subjects characterized by diverse and pre-defined physiological and demographic attributes such as age, sex, skin color, body mass index (BMI), and underlying cardiovascular conditions. During targeted sessions, the candidate device is evaluated under varying natural or induced BP changes, capturing real human variability, motion artifacts, and physiological responses. Such limited-scale human studies, although smaller in scope compared to full clinical trials, provide direct confirmation of device performance under realistic usage scenarios. When combined with laboratory-based optical simulations and phantom studies, this mixed-method approach leverages the advantages of both simulated and real-world validation strategies, offering an optimal balance between rapid laboratory qualification and critical clinical realism.

Combining these methodologies yields substantial advantages for device manufacturers, healthcare providers, and regulatory bodies. First, employing simulated signals or signals recorded from prior clinical trials dramatically reduces qualification timelines, facilitating rapid market introduction of new device models. Simulators can swiftly replicate a variety of conditions that would otherwise require extensive and costly clinical trials. Moreover, repeating identical scenarios across multiple devices using stored clinical datasets ensures consistent and reproducible validation processes, significantly enhancing regulatory acceptance and reliability. Additionally, employing tissue-mimicking phantoms adds further confidence, as qualified devices are verified against realistic human optical interaction scenarios without the complexity of real clinical environments.

In some examples, performing targeted human testing on carefully selected representative cohorts further complements benchtop testing, providing essential real-world performance data to strengthen regulatory submissions and increase user trust. By deliberately structuring such tests to include diverse skin tones, demographic variations, and specific cardiovascular profiles, device manufacturers ensure broad applicability and clinical robustness. Regulatory bodies gain reassurance that devices perform reliably across a diverse patient population without necessitating repeated large-scale pivotal trials. Economically, these combined benchtop methodologies significantly reduce development and regulatory submission costs, lowering barriers to entry for innovative devices. Small manufacturers or new market entrants can rapidly qualify their devices without prohibitive investment in full-scale clinical validation, thus accelerating innovation and market competitiveness. Furthermore, qualified devices that undergo robust benchtop testing combined with limited-scale human validation studies can achieve accelerated regulatory pathways, expediting product approval and market access.

In some examples, regulatory-mandated qualification processes are conducted entirely outside of the system 100. Regulatory agencies such as the FDA (Food and Drug Administration) in the U.S., EMA (European Medicines Agency) in Europe, and PMDA (Pharmaceuticals and Medical Devices Agency) in Japan may require independent, third-party certification of medical-grade measurement devices before they can be integrated into an ecosystem of verified devices. In such examples, the qualification process is conducted externally by accredited regulatory laboratories or notified bodies, following official standards such as ISO 81060-2 for clinical validation of BP monitors. After receiving regulatory approval and passing these external audits, the measurement device may be recognized as a verified device within the system 100, ensuring full legal and compliance adherence.

In some examples, third-party manufacturers can independently perform the qualification process before integrating their devices into the system 100. For example, a smartwatch company interested in offering calibration-free blood pressure estimation via the system 100 may conduct internal validation studies, following the predefined qualification protocol established by the system's provider. This manufacturer-led qualification process ensures that only devices meeting the system's criteria are submitted for approval, reducing the burden on the system's provider while allowing for broader adoption across diverse hardware platforms.

By enabling the qualification process to be conducted outside of the system 100, it can be ensured that measurement devices meet strict technical and regulatory standards while also facilitating greater flexibility in device integration. Whether through clinical trials, benchtop validation, regulatory certification, or manufacturer-led qualification, this approach strengthens the robustness, scalability, and compliance of the system 100, making it adaptable to a wide range of commercial, clinical, and regulatory environments.

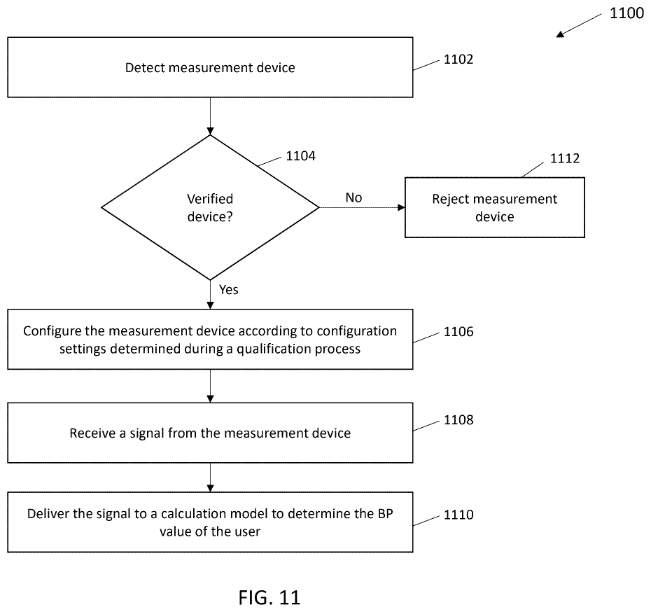

FIG. 11 illustrates a method 1100 for performing a BP determination using a verified measurement device in accordance with aspects described herein. In some examples, the application server 124 of the system 100 is configured to perform the method 1000.

At step 1102, the validation engine 134 detects a measurement device.

At step 1104, the validation engine 134 determines whether the measurement device is a verified device.

At step 1106, in response to a determination that the measurement device is a verified device, the measurement device is configured according to configuration settings determined during a qualification process. In some examples, the measurement device is configured in real-time, or at initialization stage, by applying the stored configuration settings determined during the qualification stage (e.g., the method 1000 of FIG. 10). This ensures that every subsequent physiological signal acquired from the verified measurement device adheres to the predetermined quality and shape criteria. Only after this rigorous configuration does the system 100 accept signals from the verified measurement device and deliver them to the calculation model for analysis.

For wearable devices, configuring the measurement device according to the configuration settings can be implemented in multiple ways to ensure that the data acquisition parameters align with the validated configuration. In some examples, the configuration settings are implemented via automatic over-the-air (OTA) updates, where the wearable device receives a configuration profile directly from the validation engine 134 upon being paired with the client application 112 or backend server (e.g., application server 124). This update may modify sensor parameters such as PPG sampling rate, LED intensity, photodetector gain, or signal processing algorithms to match the pre-approved settings determined during the qualification process. Another approach is to store multiple pre-configured settings in the wearable's firmware and dynamically activate the appropriate configuration based on a lookup against the verified settings in the system. This method ensures that the correct operational parameters are applied without requiring a direct firmware update, which is particularly useful for regulatory-compliant medical devices where firmware changes necessitate re-approval.

In some examples, the system 100 performs a real-time self-check upon initialization to determine whether the device already aligns with the pre-approved settings. For example, measurement device may execute a built-in diagnostic sequence where it transmits a test signal to the validation engine 134 for verification. If the signal characteristics fall within the acceptable tolerance range, no modifications are required, and the device is permitted to proceed with data acquisition. If discrepancies are detected, the validation engine 134 may either attempt to adjust the configuration automatically or alert the user 110 to update their device settings manually through the client application 112.

In some examples, the measurement device is configured via application-level control of the configuration settings. In some examples, the measurement device relies on a connected app (e.g., the client application 112) to modify real-time settings before each measurement session. For instance, a smartwatch running the client application 112 may receive an instruction from the application to adjust LED brightness, exposure duration, or analog front-end (AFE) gain levels depending on the user's environmental conditions (e.g., ambient light interference) or physiological characteristics (e.g., skin tone, perfusion index). The client application 112 then confirms with the validation engine 134 whether the adjusted settings are compliant with the pre-qualified configuration.

In measurement devices that allow for user-adjustable settings, the system 100 may include an interactive calibration step where the user 110 is prompted to select predefined sensor configurations through an interface (e.g., of the client application 112). The system 100 can then validate whether the user's selections meet the qualified criteria, preventing unapproved configurations from being used for BP estimation. This approach may be particularly relevant in regulatory-sensitive environments where manual adjustments must remain within strict compliance boundaries.

In some examples, configuring the measurement device according to the configuration settings involves adapting the built-in camera hardware and software parameters to ensure that optical signals used for PPG-based BP estimation are recorded under controlled and pre-qualified conditions. In some examples, the validation engine 134 is configured to perform an automated camera configuration through an application programming interface (API). In such examples, the validation engine 134 dynamically sets parameters such as frame rate, ISO sensitivity, exposure time, and white balance before initiating a measurement. The client application 112 ensures that these settings match the validated configuration and prevents the user from manually altering them, maintaining compliance with regulatory and clinical validation standards.

In some examples, the validation engine 134 may query the smartphone's camera capabilities to determine whether they align with the pre-approved configuration settings. Many smartphone models vary in camera sensor specifications, including differences in sensor pixel size, optical filters, or rolling shutter characteristics, which could influence the quality of PPG signal extraction. The validation engine 134 may compare the detected camera specifications with a pre-qualified list of supported configurations and either proceed with measurement if the settings are compliant or adjust the camera's parameters accordingly.

For scenarios where smartphones have varying hardware capabilities, one approach involves adaptive signal post-processing rather than modifying real-time camera settings. In such examples, the system 100 (e.g., processing engine 130 or validation engine 134) applies algorithmic corrections (e.g., motion compensation, denoising, or spectral filtering) to ensure that the extracted PPG signals remain consistent with the signals obtained during the qualification process. If a particular smartphone model exhibits known deviations due to its camera's response to light intensity, the system 100 can apply pre-defined transformation functions to align its data with the expected standard.

In some examples, a guided user experience is provided where the client application 112 instructs the user 110 to manually adjust the measurement environment to match validated conditions. For instance, if a given smartphone's flash intensity cannot be adjusted programmatically, the client application 112 may provide instructions such as “Move your finger slightly closer to the camera” or “Ensure that the measurement is taken in a well-lit environment” to compensate for any hardware limitations. Additionally, real-time quality monitoring ensures that non-compliant recordings (e.g., underexposed or oversaturated images) are either discarded or reprocessed before BP estimation.

In some examples, self-adaptive ML models are incorporated by the model engine 126 that dynamically adjust configuration settings based on the real-time performance of the recorded PPG signals. In such examples, the validation engine 134 (or the quality engine 116) continuously monitors signal quality metrics and modifies camera parameters accordingly within pre-defined regulatory constraints. For instance, if the validation engine 134 (or quality engine 116) detects excessive noise due to improper exposure settings, it may iteratively adjust the ISO and shutter speed until an optimal signal is obtained.

In some examples, the system 100 locks specific camera settings to prevent variability in measurements. For example, once a smartphone model is verified as a qualified measurement device, the client application 112 may restrict access to settings that could alter the measurement conditions. This ensures that all users of a particular device model obtain consistent and reproducible blood pressure estimations, regardless of individual user modifications.

By implementing the configuration settings on the measurement device, it can be ensured that all types of devices are configured to appropriately maintain consistency with the system's qualified settings. Whether through automated adjustments, adaptive processing, guided user instructions, or controlled firmware updates, the system 100 ensures that measurement devices operate within the pre-approved parameters necessary for reliable, clinical-grade BP estimation.

At step 1108, the application engine 124 receives a signal from the measurement device. In some examples, the signal is delivered to the processing engine 130 for pre-processing (or conditioning).

At step 1110, the signal is delivered to the BP estimation model of the model engine 126 to determine the BP value of the user 110. The BP estimation model processes the validated signal, determining and outputting an accurate, clinically-compliant BP value for the user 110. This structured verification approach significantly improves the robustness, accuracy, and regulatory compliance of BP estimation from diverse measurement devices.

At step 1112, in response to a determination that the measurement device is unverified, the measurement device is rejected. In some example, the measurement device is considered to be unverified if it is not includes in the list (or repository) of verified devices. If the measurement device is not included in the list of verified devices, the system 100 prevents its use for BP estimation. In some examples, the system 100 displays a clear message indicating that the device is not supported (e.g., via the client application 112). This functionality serves several purposes: ensuring regulatory compliance, maintaining high accuracy standards, and driving commercial adoption of verified devices or applications. In some examples, a message is sent to the application server 134 indicating that a user has attempted to (or requested to) use an unverified device. In such examples, the owner of the BP estimation model and/or the device manufacturer may be alerted that there is a need or demand to qualify the unverified device.

In some examples, when an unverified measurement device (e.g., a smartwatch, smart ring, or fitness tracker) is used to acquire PPG signals, the system validation engine 134 detects that the device has not undergone the necessary qualification process. Upon recognizing this, the device itself may display a message on its screen, such as: “This device is not certified for blood pressure monitoring. Please use a verified device for accurate readings.” If the device lacks a screen, a paired smartphone application (e.g., client application 112) may display the warning instead, preventing the user 110 from proceeding with measurements.

In some examples, when the user 110 is collecting signals via a smartphone-based PPG acquisition method (e.g., using the smartphone camera for optical signal recording), the validation engine 134 cross-checks the phone's model, camera specifications, and software version against a database of verified devices. If the smartphone model is not on the list, the client application 112 displays an alert, such as: “This smartphone model has not been validated for blood pressure estimation. Please use a verified device.” In some examples, the message is accompanied by a link directing the user 110 to a list of supported devices or an option to purchase an officially supported model.

Users of unverified devices may be directed toward verified products, driving sales and incentivizing manufacturers to seek qualification for their devices. For example, a user attempting to use an unverified smartwatch or smartphone app may be shown a message guiding them to an official online store where they can purchase a compatible device. This approach encourages users to choose devices that guarantee clinically validated BP readings, enhancing both user trust and brand credibility. In some examples, subscription-based or premium features are applied such that access to BP monitoring is restricted to users with a verified device. In this case, users attempting to access the feature with an unverified device may see an option stating: “Upgrade to a verified device to unlock medical-grade blood pressure monitoring.”

From a regulatory perspective, ensuring that only verified devices interact with the BP estimation model protects the integrity of medical-grade BP estimation. For example, in medical applications where regulatory bodies such as the FDA or CE authorities impose strict accuracy standards, preventing unqualified devices from being used mitigates legal and safety risks. In a healthcare setting, if a doctor attempts to integrate patient data from an unverified device into a clinical system, an alert may be triggered, preventing inaccurate data from entering a patient's medical records.

In enterprise or business-to-business implementations, such as insurance or remote patient monitoring platforms, insurers or healthcare providers may only reimburse users who collect data using verified devices. When an unverified device is detected, the system may display: “Your device is not eligible for insurance coverage. Please use a verified device to qualify for health benefits.” This ensures compliance with reimbursement policies while further incentivizing users to adopt approved hardware.

In some examples, signal the validation engine 143 is configured to validate the signal before delivering it to the BP estimation model (e.g., between steps 1110 and 1112). Although a measurement device may successfully pass rigorous initial qualification testing, the inherent variability in real-world operating conditions, user behavior, environmental factors, and/or physical handling can introduce unforeseen signal quality degradation. Thus, integrating a real-time signal quality validation step ensures that the BP values generated by the BP estimation model remain consistently accurate, clinically relevant, and regulatory compliant, even when used by non-expert users.

In some examples, the validation engine 134 continuously evaluates the received signals using predetermined criteria directly linked to parameters established during the initial qualification process (e.g., the method 1000 of FIG. 10). For instance, during qualification, a specific PPG waveform morphology, SNR, or expected spectral characteristics may have been established as optimal. The validation engine 134 may continuously evaluate real-time signals against these qualification-established benchmarks to confirm ongoing compliance. If deviations occur, such as reduced SNR or altered waveform shape due to improper device placement, the system 100 could automatically reject or flag these signals. By ensuring that signals continuously align with those qualified parameters, the system 100 maintains accuracy without repeated access to a clinical “ground truth.”

In some examples, the signal quality validation is based on physiological plausibility and integrity checks independent of any external reference. This approach leverages built-in heuristic evaluations of physiological coherence. For example, a recorded signal may be analyzed in real-time to confirm the presence and temporal alignment of essential physiological features like the systolic peaks, dicrotic notches, or expected frequency components within heart rate bands. If these fundamental features are absent, significantly distorted, or physiologically implausible (e.g., inconsistent pulse intervals indicating arrhythmias or noise), the system 100 may identify such recordings as invalid. This method requires no external validation reference, making it ideal for large-scale deployment.

In some examples, contextual and environmental information is combined for signal quality checks, enhancing robustness through comprehensive validation approaches. Wearable devices may incorporate motion sensors (e.g., accelerometers, gyroscopes) or ambient sensors (e.g., light sensors, temperature sensors) to infer environmental conditions and user behavior. If excessive movement, ambient light interference, or significant temperature deviations are detected, the system 100 may flag the recorded signals. For example, a smartwatch detecting erratic motion can trigger a quality validation alert, prompting the user 110 to remain still or retake the measurement under controlled conditions. Similarly, smartphones recording signals using the camera may implement real-time monitoring of ambient light conditions or user finger stability, discarding measurements if ambient light or finger placement deviates substantially from qualification-approved conditions.

In some examples, AI/ML models are trained during the qualification process to detect anomalies or atypical signal patterns. A ML algorithm trained on extensive libraries of qualified signals can assess real-time incoming signals, rapidly identifying patterns indicative of poor signal quality, interference, or user misalignment. Such algorithms may automatically assign quality scores to each recorded measurement, only allowing data surpassing defined thresholds to pass to the BP estimation model. This approach offers scalable and adaptive validation suited to both medical-grade and consumer health deployments, with performance continuously refined through ongoing data collection.

In addition to automated validation, the validation engine 134 may support user-driven or interactive validation checks. Before finalizing the measurement, the user 110 may receive real-time visual or textual feedback through the client application 112 instructing them to correct sensor placement, remain still, or alter environmental conditions. If the system 100 detects a quality concern, such as insufficient finger contact on a smartphone camera or partial loss of sensor skin contact, it provides immediate feedback, prompting corrective action. This user-centric embodiment significantly reduces false readings, enhancing overall user experience and reliability of health insights delivered.

It should be appreciated that multiple layers of validation (e.g., initial hardware-level integrity checks, physiological plausibility evaluations, machine learning-driven anomaly detection, and contextual condition verification) may be combined to comprehensively ensure signal integrity. Such layered validation schemes adapt dynamically to diverse use cases, environmental conditions, and user behaviors, providing enhanced flexibility and robustness for large-scale deployments in diverse populations and varied regulatory regions.

The use of real-time validation may also adapt dynamically to different regulatory contexts. For example, systems deployed in regions with stricter regulatory accuracy requirements may enforce tighter validation criteria, discarding signals at lower noise thresholds or stricter physiological plausibility standards. Likewise, deployments for consumer wellness contexts might apply slightly relaxed criteria, balancing accuracy against user convenience and measurement success rates. This adaptive approach allows seamless scaling of the system 100 across regulatory territories, clinical contexts, and consumer environments without compromising regulatory compliance or accuracy requirements.

In some examples, continuous signal quality validation enables ongoing monitoring and adaptive device management strategies. For example, continuous detection of reduced signal quality across a large user base could trigger device-specific alerts or firmware updates, addressing widespread hardware issues or algorithmic adjustments proactively. This aggregated data analysis can provide valuable real-world performance insights, informing continuous device qualification refinements, improving user instructions, or guiding targeted training for specific user groups.

In some examples, the system 100 identifies personal characteristics of the user 110 and delivers these characteristics to the BP estimation model alongside the physiological signal to enhance the accuracy of BP estimation. The BP estimation model may use additional information beyond the physiological signal to contextualize and refine BP predictions, particularly in calibration-free approaches where no individualized baseline is established. These personal characteristics may include, but are not limited to, age, weight, height, biological sex, ethnicity, skin tone, body mass index (BMI), wrist circumference, vascular compliance metrics, location, time zone, sleep cycle information, medication intake status, smoking habits, hydration levels, caffeine consumption, and genetic predisposition to hypertension. In some examples, these characteristics are manually provided by the user 110 through the client application 112 during initial system setup or periodically updated through an in-app profile section. The system 100 may prompt the user 110 to input relevant data and store it securely for future use. This implementation ensures user control over personal information while maintaining transparency in the data-sharing process.

In some examples, the system 100 automatically fetches certain characteristics from the device 102a, 102b. For example, a smartphone-based implementation may retrieve height, weight, and age from the user's Health Profile stored on the smartphone's native health data repository, such as Apple Health on iOS devices or Google Fit on Android devices. These platforms aggregate health-related data from multiple apps and connected devices, ensuring that the system 100 has access to the most up-to-date and consistent personal health information.

In some examples, the system 100 integrates with third-party health platforms, including cloud-based electronic health records (EHRs) maintained by healthcare providers. Through secure authentication protocols and APIs, the user engine 128 may query EHR databases to obtain clinically verified personal characteristics of the user 110, such as biometric measurements recorded during previous medical visits, current medication regimens that may influence blood pressure, and historical cardiovascular risk assessments. This approach may be particularly relevant for regulated medical applications, where integrating clinical-grade data can enhance diagnostic accuracy.

In some examples, wearable devices (e.g., user device 102b) continuously monitor certain physiological characteristics that may act as proxies for relevant personal attributes. For example, a smartwatch with a built-in body composition sensor may provide real-time estimations of BMI, hydration levels, and body fat percentage, which can be transmitted to the BP estimation model along with the primary physiological signal. Similarly, sleep trackers may provide information on circadian rhythms, helping the model adjust for expected diurnal variations in BP.

The system 100 may also utilize geolocation data to infer contextual personal characteristics. For instance, altitude information obtained from GPS or barometric sensors may help adjust BP predictions in users living at high elevations, where baseline arterial pressure differs from sea-level populations. Additionally, real-time weather data can indicate external temperature conditions, allowing the model to adjust for expected vasoconstriction or vasodilation effects.

In privacy-sensitive applications, the system 100 may implement federated learning techniques or edge computing methods that process and anonymize personal characteristics locally on the user's device before delivering only essential parameters to the model. This ensures data security and regulatory compliance, particularly in jurisdictions with strict data protection laws such as GDPR in Europe or HIPAA in the United States.

Regardless of the data acquisition method, the system 100 can ensure that the identified personal characteristics are correctly formatted and structured before being transmitted to the BP estimation model. The data may be standardized into predefined input categories, normalized against population-wide reference datasets, or weighted based on their statistical influence on BP estimation. By incorporating a diverse range of personal characteristics, the system enhances both the generalizability and accuracy of its calibration-free BP estimation, optimizing performance across varied user demographics and real-world conditions.

In some examples, the device qualification process enables a centralized-party to maintain a regulatory-approved, calibration-free BP estimation model operating within a backend infrastructure, capable of generating medically reliable BP values from PPG signals from various measurement devices. To integrate third-party measurement devices, the centralized-party may conduct a comprehensive qualification procedure, involving benchtop testing and rigorous validation. During this qualification procedure, the centralized-party may carefully evaluate the waveform shape and quality of PPG signals captured by third-party wearables, ensuring they consistently meet predefined technical specifications. Configuration settings, such as light intensity, pulse duration, wavelength, sampling rates, and analog front-end parameters, are precisely determined and stored for each qualified third-party device.

Upon successful qualification, the centralized-party incorporates the verified third-party devices into its system, allowing their users direct access to the centralized-party's calibration-free BP estimation models through a cloud-based API service. In some examples, the centralized-party distributes an SDK to third-party manufacturers, enabling seamless integration and configuration of their wearables for communication with the backend API service. Users of qualified devices may then seamlessly transmit their PPG signals to the centralized-party's computational model via the provided SDK. The centralized model processes the incoming validated signals and returns regulatory-approved, clinically validated BP values back to the user.

In some examples, the centralized API-driven service enables the broad-scale delivery of medically approved, calibration-free BP monitoring to a much larger user base than would be possible through the use of a single party's own measurement devices. As described above, third-party manufacturers gain immediate access to a robust, medically compliant BP estimation capability without the prohibitive costs, complexity, or delays involved in running dedicated clinical trials or gaining individual regulatory approvals.

In some examples, all processing operations, including signal acquisition, signal validation, feature extraction, and BP computation, occur entirely on the measurement device itself, such as a smartwatch or a wristband. This configuration may be ideal for a fully autonomous medical-grade device where data security and offline usability are crucial. By running the BP estimation model locally, users can receive real-time feedback without relying on external servers, reducing latency and ensuring privacy compliance under regulations such as GDPR or HIPAA. This setup benefits regulated medical markets where cloud-based processing may introduce data residency concerns.

In some examples, all blood pressure estimation processes can be performed on the device, but the final BP result is securely transmitted to a cloud service for data logging, regulatory auditing, and health analytics. This ensures compliance with medical recordkeeping regulations while maintaining user privacy, as only processed BP values, not raw sensor signals, are transmitted. This embodiment may be particularly attractive for clinical-grade remote patient monitoring applications.

In some examples, a fully remote cloud processing configuration is used, where the measurement device, such as a smartwatch or smartphone camera, only captures raw physiological signals and transmits them to the application server 124 for all processing steps.

The remote processor validates the signal, applies the BP estimation model, and delivers results back to the user's measurement device. This configuration enables scalability for consumer-grade wellness applications, as updates to the BP estimation algorithms can be deployed centrally without requiring firmware updates to millions of devices. It also allows cross-platform compatibility, where multiple devices from different manufacturers can leverage the same cloud-based model without local computational constraints.

In some examples, a hybrid processing configuration is used to enhance efficiency by having the measurement device perform basic preprocessing, such as noise reduction, signal normalization, and feature extraction, before transmitting the cleaned data to the application server 124 for final blood pressure estimation. This balances computational efficiency and real-time performance by reducing the data burden, minimizing transmission costs, and lowering power consumption. It may be particularly useful for low-power wearables, such as fitness bands with limited processing capabilities, while still ensuring high-quality BP computations through powerful server-side AI models.

In some examples, an edge computing configuration is used where intermediary devices such as smartphones, tablets, or dedicated gateways perform the first stage of signal validation before sending the refined data to the application server 124. For instance, a smartwatch may capture PPG signals and transfer them to a paired smartphone that executes initial signal validation, then forwards the data to the application server 124 for BP estimation. This method enhances efficiency by reducing bandwidth consumption and enables low-latency processing when a direct connection to cloud services is limited.

Some regulatory frameworks, such as EU MDR or US FDA medical device approvals, require that processing remains within a specific regulatory jurisdiction, such as the EU or US. As such, compliance can be ensured by processing signals in a localized cloud infrastructure rather than a globally distributed system. In some examples, the system 100 dynamically routes user data to an approved regional server (e.g., application server 124), ensuring that patient data does not leave its designated regulatory territory while still leveraging cloud-based model updates and compliance requirements.

In some examples, on-device processing is combined with optional cloud-assisted model updates. Some regulatory jurisdictions allow adaptive AI/ML models to improve performance over time. In such examples, the BP estimation model may run entirely on the measurement device, but the cloud periodically analyzes aggregated, anonymized data to improve algorithm performance. Updated models may then be pushed back to devices via secure over-the-air updates. This configuration ensures continuously improving performance while maintaining regulatory compliance for locked-down medical software.

In some examples, a distributed processing model enables multiple IoT devices to contribute different data streams for health monitoring. For example, a smart scale, smartwatch, and smartphone may all provide data such as weight, PPG, and motion tracking, with distributed processing determining BP values using all available signals. This configuration may be useful for longitudinal health monitoring and integrating BP estimation into broader health analytics platforms. In some examples, real-time processing on mobile apps is combined with optional server-based validation. In such examples, the BP estimation model runs within a smartphone app (e.g., client application 112), allowing immediate BP estimation without requiring an internet connection. However, if a questionable signal is detected, the smartphone can upload the raw data to a cloud-based validation service (e.g., the validation engine 134), which applies advanced algorithms to verify accuracy and return a revised BP estimate. This hybrid approach provides instant feedback for users while maintaining the ability to leverage high-performance cloud-based computation when needed.

By offering flexible architectures for processing BP estimation and device qualification, the system 100 ensures it can be widely adopted across various industries, including wearable technology, telemedicine, fitness tracking, and clinical monitoring, while maintaining the necessary regulatory and performance standards for each use case.

### Model Training

A model training framework is used to develop and validate the BP estimation model for non-invasive, calibration-free BP estimation. In some examples, the BP estimation model is trained on a first dataset of physiological signals and refined using a second dataset annotated with clinically recognized reference BP values obtained from a first cohort. The refined model is subsequently validated against a second cohort to ensure compliance with specific accuracy criteria. This structured and rigorous approach ensures clinical reliability, generalizability across diverse populations, and robust performance in real-world conditions.

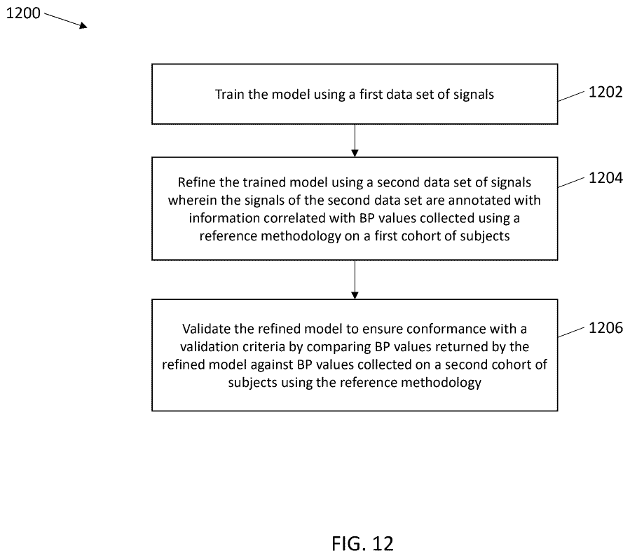

FIG. 12 is a method 1200 for training a BP estimation model in accordance with aspects described herein. In some examples, the method 1200 is performed, at least in part, by the model engine 126 of the application server 124. In some examples, the method 1200 corresponds to a structured and scalable methodology for developing a computational model capable of non-invasive, calibration-free BP estimation. Unlike some training approaches that rely exclusively on small-scale, high-quality datasets collected in controlled clinical settings, the training approach of method 1200 introduces a multi-stage process that balances the need for vast amounts of diverse training data with the requirement for strict clinical accuracy.

By employing a two-phase training strategy—first training on a large, weakly labeled (or unlabeled) dataset and then refining the model using a smaller, highly controlled dataset—the method 1200 enables the creation of a robust and generalizable BP estimation model. This methodology addresses a longstanding challenge in the field, where the collection of large-scale, high-quality BP datasets has been prohibitively expensive, time-consuming, and logistically unfeasible. By leveraging real-world data collection at scale in combination with high-quality fine-tuning, the method 1200 ensures that the trained model can accurately estimate BP across a broad population while meeting the stringent accuracy standards required for regulatory approval.

At step 1202, the model engine 126 trains the BP estimation model using a first data set of signals. In some examples, the first data set of signals are used in a first phase (or stage) of the training process. The first phase of the training process involves developing a foundational model (e.g., the BP estimation model) trained on a vast dataset of physiological signals, such as PPG waveforms, recorded in real-world, unconstrained conditions. These datasets may be gathered from a large number of users who use non-invasive measurement devices to monitor their health. Unlike some training approaches that rely exclusively on small, highly curated datasets, this first phase allows the BP estimation model to develop a broad understanding of the relationship between PPG signal characteristics and BP fluctuations across a diverse population. However, because this dataset is collected in uncontrolled environments, it may contain inherent inaccuracies, variations in signal quality, and inconsistencies in reference BP values. In some examples, the reference BP values in the first dataset are obtained from oscillometric cuffs, which, while commonly used in consumer and clinical settings, are not considered the gold standard in medical research. Consequently, while the BP estimation model benefits from learning from a large dataset, further refinement is provided to achieve the level of accuracy necessary for medical-grade BP estimation.

In some examples, the first dataset is collected using scalable methodologies that require at most a minimal level of clinical supervision, ensuring rapid, cost-effective, and diverse data accumulation. This approach enables the dataset to scale to millions of data points across a wide range of real-world use cases, making the BP estimation model robust, widely applicable, and commercially viable. The requirement for minimal supervision ensures that the BP estimation model is trained on realistic data, while still maintaining enough control to ensure high-quality annotations and reliability.

Minimal clinical supervision refers to flexible oversight models that range from completely unsupervised user-driven data collection to lightly monitored clinical studies and healthcare-driven campaigns. Unlike fully controlled clinical trials that require the physical presence of trained professionals for every measurement, minimal supervision allows for decentralized, scalable, and cost-efficient data acquisition while still ensuring sufficient control to maintain dataset reliability. Depending on the use case, different levels of supervision may be implemented, each with its advantages and trade-offs. Below, different levels of clinical supervision are described that can be applied in varying scenarios, enabling rapid, large-scale data collection without sacrificing data integrity.

Scenario 1: No Supervision—Direct-to-Consumer, Fully Unsupervised Data Collection. In this scenario, users autonomously record PPG and BP signals using commercially available devices sold over-the-counter (OTC) or through consumer electronics distribution channels. No healthcare professional is involved in the data collection process. The data collection system is embedded into consumer devices (e.g., user devices 102a, 102b) such as smartwatches, rings, fitness bands, smartphones, and wearable BP monitors. The user is responsible for performing their own measurements, and data is automatically uploaded to cloud servers for aggregation (e.g., application server 124). For example, a wristband may be used to automatically collect PPG and BP data and transmits it to the dataset (e.g., stored in model data database 136a). This scenario is advantageous because it scales to millions of users with no extra operational costs, provides real-world, naturalistic data from diverse populations in everyday conditions, and encourages continuous long-term monitoring, increasing dataset size and diversity. In some examples, data validation mechanisms are used to filter low-quality or biased data points.

Scenario 2: Basic Supervision—Decentralized Clinical Investigations with Remote Oversight. In this scenario, users still perform measurements at home, but in the context of an ongoing clinical investigation where a clinical investigator remotely monitors and validates the collected data. Participants self-record data using wearable or smartphone-based systems. A clinical investigator supervises the protocol remotely, ensuring consistency in data collection across participants. The investigator may not be present at each recording but will review data logs, metadata, and consistency reports to detect anomalies or ensure compliance. For example, a study with 10,000 participants using their smartphones to record PPG and BP signals daily, with researchers ensuring proper adherence through periodic validation calls and data integrity checks. This scenario is advantageous because it maintains scalability while adding some degree of validation and reduces costs compared to traditional in-clinic studies while preserving better control over dataset integrity. In some examples, active engagement strategies (e.g., reminders, alerts, rewards, etc.) are used to maintain data consistency. In some examples, a structured methodology is used to handle missing or low-quality data.

Scenario 3: Healthcare Campaigns—On-Site Data Collection with Healthcare Professionals. In this scenario, large-scale campaigns with trained healthcare professionals collect data from subjects at various locations, such as pharmacies, clinics, public events, or community outreach sites. Medical personnel actively collect BP and PPG data using clinical devices. These professionals may operate outside hospital settings, conducting population-wide screenings in workplaces, schools, malls, or even in mobile health vans on the street. Data is collected following a standardized protocol but allows for scalability by eliminating the need for a fully controlled hospital setting. For example, a campaign to collect 50,000 BP readings from wearable devices at pharmacies and public health kiosks, where pharmacists ensure correct usage but do not conduct full medical supervision. This scenario is advantageous because it provides a balance between high-quality data and scalability, ensures that device usage is correct and that signals meet baseline quality criteria, and enables data collection from diverse demographics, including those who do not own BP monitors at home.

Scenario 4: Fully Supervised Clinical Trials with Controlled Conditions. In this scenario, data is collected in a fully controlled environment where subjects undergo BP and PPG recordings under strict supervision of trained healthcare professionals. Subjects are enrolled in formal clinical trials, typically conducted in hospitals, research labs, or regulatory evaluation centers. BP readings are taken using gold-standard methods, such as double-auscultatory BP measurement with two trained professionals using a stethoscope. Participants are monitored in controlled conditions (e.g., sitting in a defined position for 5 minutes before measurement). For example, a 500-patient study where BP measurements are taken under ISO 81060-2 conditions, with every step documented and reviewed by clinicians. This scenario is advantageous because it produces the highest-quality reference dataset and provides data that meets regulatory and medical validation requirements.

Scenario 5: Hybrid Approaches—Combining Supervision Levels for Optimal Scalability. In this scenario, the system 100 allows for any mixture of the above approaches to balance scalability, cost, and data quality. Examples of hybrid methodologies include: mass consumer data collection (i.e., Scenario 1) with selective clinical validation (i.e., Scenario 2) to improve dataset reliability, pharmacy-based campaigns (i.e., Scenario 3) with a subset of users participating in controlled clinical trials (i.e., Scenario 4) to ensure high-quality validation data, and remote clinical trials (i.e., Scenario 2) combined with on-site testing at healthcare facilities (i.e., Scenario 3) to create a broad, representative dataset.

In some examples, the first dataset includes pulsatility signals from a variety of devices and sensors. In some examples, the sensors vary in both sensor type and body location. Such signal flexibility ensures that the foundational BP estimation model benefits from a broad and diverse dataset, enhancing generalizability across devices, users, and physiological conditions.

Below are multiple example datasets demonstrating how different sources of pulsatility signals can be leveraged in constructing the first dataset. In some examples, the datasets vary based on sensor and/or device type. In some examples, the datasets vary based on body location. It should be appreciated that the following examples are not intended to be limiting and may be used as standalone datasets or sub-datasets that are combined to produce a complete dataset.

Example 1: Single-Sensor Dataset. The first dataset consists of signals from thousands of users wearing a first device type having a PPG sensor located at the wrist. This dataset provides consistent wrist-based pulsatility signals, collected across diverse user demographics and environmental conditions.

Example 2: Dual-Sensor Dataset. The first dataset is expanded to include wrist PPG signals from the first device type and video-based PPG signals from a second device type. The second device type utilizes a smartphone camera to record pulsatility signals when the user places their fingertip over the camera lens. of signals from thousands of users wearing a device having a PPG sensor located at the wrist. This multi-source dataset enhances the robustness of the model by incorporating optical signals collected under different lighting conditions and body positions.

Example 3: Multi-Sensor Dataset. The first dataset consists of signals from the first device type (i.e., wrist PPG), second device type (i.e., video PPG), and a third-party wrist-worn PPG sensor. This dataset allows the system to learn patterns across different hardware platforms, ensuring model adaptability for multiple device ecosystems.

Example 4: Multi-Wavelength Sensor Dataset. The first dataset includes PPG signals obtained from sensors using different wavelengths (e.g., green PPG, red PPG, and infrared PPG). The inclusion of different wavelengths improves signal quality by accounting for varying skin tones, perfusion levels, and motion artifacts.

Example 5: Multi-Modality Dataset. The first dataset integrates PPG signals from wrist, fingertip, and smartphone cameras, combined with ECG data from wearables (e.g., smartwatches with ECG capabilities) for cross-validation of pulsatility waveforms. The system learns the correlation between PPG waveforms and ECG-derived heart activity, improving BP estimation accuracy.

Example 6: Wearable Ring Dataset. The dataset includes signals from PPG sensors embedded in wearable ring devices (e.g., Oura Rings, smart rings, etc.). Rings provide a different vascular measurement site than the wrist, offering higher signal stability and lower motion artifacts.

Example 7: Ear-Worn Device Dataset. The dataset is expanded to include signals from earbud-integrated PPG sensors (e.g., hearables, medical-grade earpiece PPG sensors, etc.). The ear provides a highly stable site for pulsatility detection, as it is less susceptible to motion artifacts compared to wrist-based sensors.

Example 8: Headbands or Smart Glasses Dataset. The dataset includes PPG signals recorded from the temple region using smart glasses or headbands. This dataset adds another vascular measurement location, useful for cross-verification of signals collected from other body sites.

Example 9: Clinical Pulse Oximeters Dataset. The dataset incorporates medical-grade pulse oximeters attached to the fingertip, toe, or ear. These signals serve as high-quality reference data to improve model accuracy and detect signal discrepancies present in consumer-grade wearables.

Example 10: Chest-Worn Devices Dataset. The dataset includes signals that are collected from chest-based PPG sensors integrated into smart patches, chest straps, or smart clothing. The chest offers a centralized vascular site, making these signals valuable for high-fidelity physiological monitoring.

Example 11: Wrist PPG Only (Homogeneous Dataset). All signals are collected from wrist-based wearables, ensuring a consistent dataset that minimizes variability due to sensor placement.

Example 12: Fingertip PPG (Smartphone & Medical Pulse Oximeter). The dataset consists of signals from fingertip PPG recordings, including smartphone-based PPG and hospital-grade pulse oximeters.

Example 13: Combination of Wrist and Fingertip PPG. The dataset incorporates both wrist-based and fingertip-based PPG, allowing the model to understand vascular waveform transformations across different locations.

Example 14: PPG from Multiple Body Sites Simultaneously. The dataset includes synchronized recordings from multiple body locations, such as wrist, ear, and fingertip, to better capture individual variations in vascular responses.

Example 15: PPG from Armband-Based Devices. PPG signals collected from upper-arm armbands are included, offering another physiological site that differs from wrist-based measurements.

Example 16: PPG from Smart Footwear or Toe-Mounted Sensors. The dataset incorporates toe-based PPG measurements, collected using smart socks or pulse oximeters attached to the toe, providing signals from peripheral circulation.

Example 17: PPG from Smart Clothing. Smart textiles embedded with optical sensors record pulsatility signals from larger vascular beds (e.g., chest or thigh). This dataset contributes to expanding signal variability and improving accuracy across different body locations.

Example 18: PPG from Smart Helmets for Athletes. The dataset includes PPG signals from helmets designed for athletes, where forehead-based vascular monitoring provides an alternative site for pulsatility signal extraction.

Example 19: PPG from Non-Contact Sensors (Radar or Optical Reflection-Based). The first dataset includes signals from non-contact sensors that use radar or long-distance optical PPG to measure blood flow patterns remotely (e.g., automotive or hospital-based systems for passive monitoring).

Example 20: PPG from Emergency Medical Sensors. The dataset incorporates PPG readings from emergency response settings, where sensors integrated into ambulance patient monitors record pulsatility signals under varying stress conditions.

The range of these examples demonstrates how the first dataset can integrate diverse pulsatility signals collected from different sensor/device types and body locations. This ensures that the foundational BP estimation model is not restricted to a single data source, but instead benefits from multi-source training, leading to a more adaptable, generalizable, and clinically robust model. By leveraging wearables, smartphones, clinical sensors, and novel body locations, the BP estimation model can improve its accuracy, applicability, and resilience across a wide range of real-world use cases.

To enhance the robustness and generalizability of the BP estimation model, the first dataset may be augmented using synthetic data generated by either a digital or an analog simulator. This augmentation approach extends beyond collecting data from real users, allowing the BP estimation model to learn from a broader range of physiological conditions, including both healthy and pathological cases. Additionally, randomized augmentation techniques may be applied to existing datasets to introduce variations and ensure the model is trained on diverse signal patterns.

An analog simulator is a physical device that mimics real-world PPG signals by generating optical signals that are fed directly into a sensor, such as a PPG photodiode in a wearable device. This approach allows controlled and repeatable testing without requiring human subjects. In some examples, the analog simulator is an electromechanical optical signal generator. An electromechanical optical signal generator is a mechanical system with artificial tissue layers that replicates the light absorption and reflection properties of human skin. These generators can be used to produce pulsatility signals that resemble real PPG waveforms. The generator may be configured to generate pulses of varying amplitudes and frequencies, simulating the cardiovascular dynamics of different individuals, including hypotensive and hypertensive cases. In some examples, the analog simulator is a programmable LED-based optical simulator. A programmable LED-based optical simulator may be composed of infrared, red, and green LEDs that modulate light output in synchronization with an artificial heart rate signal. In some examples, a sensor detects these optical variations as if they were generated by real blood flow. In some examples, the analog simulator is configured to provide the playback of pre-recorded clinical signals. The analog simulator may reproduce real PPG waveforms recorded from clinical trials, ensuring that known, high-quality datasets can be fed into the system without requiring new human participants. In some examples, the analog simulator is configured to provide augmented analog signals with physiological variability. The analog simulator can apply micro-modulations to the optical signals, introducing synthetic motion artifacts, changes in perfusion levels, or skin tone variations to test the system's resilience to real-world scenarios.

A digital simulator generates fully synthetic pulsatility signals in a virtual environment without requiring any physical sensor interaction. In some examples, synthetic signals can be created using computational cardiovascular models. Such computational models simulate the cardiovascular system, including arterial compliance, blood viscosity, and pulse propagation to generate synthetic PPG signals. These models can be used to generate signals for different pathophysiological states (e.g., hypertensive patients, arrhythmias, poor peripheral circulation). The generated signals can be stored as digital waveforms, processed as if they were real recordings, and fed into the AI/ML pipeline. In some examples, synthetic signals are created via the reconstruction of synthetic PPG signals from clinical or noisy real-world data. The simulator extracts fundamental waveform characteristics from pre-recorded real-world signals and generates similar but distinct waveforms through computational modeling. This approach enables augmentation with slightly modified versions of real PPG data, expanding the training dataset without requiring new user acquisitions.

Once real, simulated, or synthetic signals have been gathered, additional augmentation techniques may be applied to increase dataset diversity. For example, the model engine 126 may add random noise to the dataset. In some examples, introducing Gaussian noise, white noise, or realistic sensor noise allows for simulation of measurement imperfections. The BP estimation model learns to distinguish between real pulsatile signals and noise contamination, improving robustness. In some examples, the model engine 126 is configured to introduce physiological variability into the dataset. In some examples, signal morphology is modified to reflect age, sex, and vascular health variations. Adjusting pulse amplitude, shape, and frequency allows for simulation of different physiological states. In some examples, the model engine 126 is configured to apply motion artifacts and recovery strategies. For example, the model engine 126 may superimpose synthetic motion artifacts derived from real-world movement patterns. In some examples, the model engine 126 is configured to test whether the BP estimation model can filter out and reconstruct clean pulsatility waveforms in real-world use cases. In some examples, the model engine 126 is configured to resample and temporally warp signals in the dataset. For example, signal durations may be altered to mimic variations in heart rate and respiration-driven changes in PPG signals. In some examples, the model engine 126 is configured to simulate environmental lighting conditions. For example, the model engine 126 may adjust signal intensity to reflect changes in ambient light conditions for PPG sensors embedded in measurement devices.

To complement the augmentation of pulsatility signals, the BP reference values used in model training may be artificially expanded. In some examples, the model engine 126 is configured to reproduce reference BP values for simulated signals. When synthetic signals are generated from a computational cardiovascular model, the associated BP values may also be simulated based on physiological models. This ensures that the BP estimation model can learn from an extended range of BP variations, including extreme hypertension and hypotension scenarios. In some examples, the model engine 126 is configured to randomize BP values for robust model training. Instead of training the BP estimation model with fixed BP values, slight variations may be introduced to account for measurement errors in real-world BP devices. This allows the BP estimation model to tolerate uncertainties in manual auscultatory readings and oscillometric BP variations. In some examples, the model engine 126 is configured to generate BP values based on statistical distributions. If a dataset contains insufficient coverage of certain BP ranges, additional synthetic BP values may be generated based on the statistical properties of the existing dataset. This ensures that the model is not biased towards normal BP values but also learns from extreme cases.

The ability to augment the first dataset with synthetic, simulated, and randomized signals significantly improves the adaptability and generalizability of the BP estimation model. By leveraging both analog and digital simulators, the BP estimation model can train on a broad range of pulsatility signals, covering conditions that might be rare or difficult to capture in a real-world dataset. Additionally, applying signal and BP augmentation techniques ensures that the BP estimation model remains robust against real-world measurement inconsistencies, making it well-suited for large-scale deployment in medical and consumer settings.

At step 1104, the model engine 126 refined the trained BP estimation model using a second data set of signals. In some examples, the second data set of signals are annotated with information correlated with BP values collected using a reference methodology on a first cohort of subjects. In some examples, the second data set of signals are used in a second phase (or stage) of the training process. The second phase may be referred to as a “fine-tuning step.” In some examples, the second data set of signals is a smaller dataset that has been collected under strict clinical conditions. This dataset may be obtained in highly controlled experimental settings, following but not being limited to the conditions outlined in ISO 81060-2, which establishes rigorous standards for BP measurement validation. Such conditions require that the subject remains seated and relaxed for several minutes in a precise position before measurement. The standard also prescribes the use of a double-auscultatory reference method, in which two trained medical professionals independently listen to Korotkoff sounds via a stethoscope to determine systolic and diastolic BP.

This methodology eliminates the potential biases and inaccuracies associated with automated oscillometric cuffs and ensures that each measurement is performed according to the highest regulatory and scientific standards. By training on the second dataset, the BP estimation model is able to correct for inaccuracies present in the foundational version of the model and align its estimations with the reference values required for regulatory compliance. The structured two-phase training process, enables the development of an accurate, generalizable, and scalable BP estimation system that can function without requiring calibration.

At step 1106, the model engine 126 validates the refined BP estimation model to ensure conformance with a validation criteria. In some examples, the validation process includes comparing BP values retuned by the refined model against BP values collected on a second cohort of subjects using the reference methodology. The validation process ensures that the refined BP estimation model meets predefined accuracy criteria before deployment. In some examples, the validation process explicitly compares the model's output to BP values obtained from a second cohort of subjects under controlled clinical conditions. In some examples, the second cohort of subjects differs from the first cohort of subjects used in step 1104. The validation process ensures that the model's predictions conform to strict performance benchmarks. The validation process not only ensures compliance with medical device regulations but also guarantees that the BP estimation model remains reliable across different populations, device types, and physiological variations.

In some examples, the validation engine 134 applies validation criteria that ensures the trained BP estimation model meets established accuracy standards for non-invasive BP estimation. Specifically, the validation criteria may comprise a bias of less than 5 mmHg and a precision within either 8 mmHg or 10 mmHg, aligning with widely recognized regulatory requirements for BP monitors. These validation criteria ensures that the model's predictions remain clinically reliable and suitable for deployment in both medical and consumer applications.

Several regulatory agencies and standardization bodies have defined validation protocols that outline the acceptable accuracy thresholds that BP monitoring systems must meet before they can be approved for clinical or consumer use. Such validation standards and protocols may include, for example: ISO 81060-2: 2018—Non-Invasive Sphygmomanometers for Automated BP Measurement, AAMI/ESH/ISO 81060-2: 2022—Updated Standard for BP Measurement Validation, IEEE 1708-2014—Standard for Wearable Cuffless BP Monitors, ESH-IP 2010—European Society of Hypertension International Protocol, BHS Protocol—British Hypertension Society Standard, FDA 510(k) Clearance—US Medical Device Validation for BP Monitors, Health Canada BP Device Validation Criteria, and PMDA Approval—Japanese Blood Pressure Device Validation.

In some examples, the validation engine 134 is configured to comply with any number of validation protocols, allowing for adaptability based on the target market and regulatory requirements. The configuring the validation engine 134 may include selecting an appropriate regulatory framework based on the intended market (e.g., FDA 510(k) for the U.S., ESH-IP for Europe, PMDA for Japan), training and validating on diverse cohorts that meet the demographic and statistical requirements of the chosen regulatory standard, ensuring the trained model achieves bias and precision requirements, following the established performance thresholds, and testing across different scenarios, including resting, ambulatory, postural variations, and stress conditions, to meet real-world validation protocols.

As described above, the first dataset may be weakly labeled and the second dataset may be fully labeled (or annotated). In some examples, the first dataset is annotated with BP values obtained from various sources. These BP annotations provide a reference for training, validating, and refining the BP estimation model, ensuring that the pulsatility signals collected from different users are correlated with clinically relevant BP measurements. The annotations may be derived from multiple methodologies, including automated oscillometric devices, manual measurements, health data integrations, and clinical trials.

Below are multiple examples of how BP reference values can be obtained and integrated into the first dataset. It should be appreciated that the following examples are not intended to be limiting and may be used as standalone schemes or partial schemes that are combined to produce a complete scheme.

Example 1: Automated Oscillometric BP Measurements Integrated with PPG Systems. In some examples, a PPG-based wearable device includes an integrated oscillometric BP measurement system. For example, a device may combine a wrist-worn PPG sensor with an oscillometric cuff, enabling seamless simultaneous PPG and BP data collection without requiring user intervention. The BP values may be automatically stored alongside the corresponding PPG waveforms, ensuring high-quality data annotation.

Example 2: Automated BP Cuffs Used Independently by the User. Users may own an automated BP cuff that is not integrated into the system 100. The system 100 may allow the user to manually input BP readings whenever they perform a cuff measurement while wearing their PPG-enabled device. The BP values may be automatically stored alongside the corresponding PPG waveforms.

Example 3: BP Measurements Performed in Pharmacies. Many pharmacies offer free BP checks using clinically validated oscillometric monitors. The system 100 may allow users to input pharmacy-based BP readings manually or via a barcode scan of the pharmacy receipt. The BP values may be automatically stored alongside corresponding PPG waveforms.

Example 4: BP Measurements Taken During Routine Doctor Visits. Users may receive BP checks during annual physical exams, specialist visits, or emergency care visits. The system 100 may allow for manual entry or automated data transfer from electronic health records (EHRs) when integrated with a medical provider's platform. The BP values may be automatically stored alongside corresponding PPG waveforms.

Example 5: BP Readings from Home Health Visits and Nurses. Home healthcare professionals, such as nurses or caregivers, may take BP readings using auscultatory or oscillometric methods. These values can be uploaded via a home health monitoring app or caregiver portal. The BP values may be automatically stored alongside corresponding PPG waveforms.

Example 6: Manual BP Readings Performed in Clinical Trials. Clinical trials often use double-auscultatory BP readings (e.g., two professionals using a stethoscope for validation). The system 100 can annotate PPG signals with high-accuracy reference values obtained in such controlled conditions.

Example 7: Automated BP Readings from Wearables with Cuff-Based Inflation. Some smartwatches and wearables include inflatable micro-cuffs for BP estimation (e.g., Omron HeartGuide). The system 100 can collect these readings and align them with PPG signals recorded at the same time.

Example 8: BP Measurements from Invasive Arterial Line Monitoring. In hospital settings, arterial catheters provide continuous real-time intra-arterial BP monitoring. If available, this gold-standard BP data can be annotated with simultaneous PPG waveforms from patients in intensive care units (ICUs).

Example 9: Continuous BP Monitoring from Tonometric Devices. Some non-invasive medical-grade systems, such as applanation tonometry, continuously measure BP at the radial or carotid artery. These values can be synchronized with PPG data to enhance accuracy.

Example 10: BP Data from Health Kit Integrations. The system 100 may integrate with Apple HealthKit, Google Fit, Samsung Health, or third-party medical apps to retrieve BP readings automatically.

Example 11: BP Readings from Public or Workplace BP Kiosks. Some workplaces and shopping centers have BP kiosks, where users can check their BP and receive a printed or digital report. In some examples, users can scan a QR code or manually enter values into the system 100 for annotation.

Example 12: BP Readings from Remote Patient Monitoring (RPM) Programs.

Some users participate in RPM programs where BP data is automatically sent to their doctor. The system 100 can connect to these programs via API integrations, allowing real-time annotations.

Example 13: BP Readings from Elderly or Assisted Living Facilities. Many elderly care homes take scheduled BP readings for residents. These can be recorded and linked to wearable-based PPG recordings for additional data insights.

Example 14: BP Readings from Blood Donation Centers. Blood donors often have BP taken before and after donation. The system 100 may allow integration with blood bank databases to annotate PPG signals with donation-day BP values.

Example 15: BP Readings from Sports Science and Athletic Testing Labs.

Athletes and fitness enthusiasts may undergo stress testing and physiological evaluations that include BP checks. The system 100 may allow these professional BP readings to be used for training the BP estimation model.

Example 16: BP Readings from Emergency Medical Services (EMS) Data.

Emergency responders frequently take BP measurements on-site or in ambulances. If a user is involved in emergency care, their BP readings may be retrospectively linked to their PPG data.

Example 17: BP Readings from Personal Health Journals and Logs. Some users track BP manually in a personal health journal or a spreadsheet. The system 100 may enable entry of these logs, maintaining user-based tracking over time.

Example 18: BP Readings Captured During Surgery or Anesthesia. BP is continuously monitored during surgical procedures and under general anesthesia. These high-frequency readings can be aligned with PPG signals recorded from surgical monitors.

Example 19: BP Readings from Controlled Environmental Studies. Research studies on sleep, stress, temperature exposure, and exercise physiology often record BP under unique conditions. In some examples, these datasets provide specialized annotations that expand the model's exposure to edge cases.

Example 20: BP Readings from Smart Home Devices. Smart home health devices may passively measure BP using smart mattresses, non-contact radar sensors, or pressure-sensitive flooring. These BP values may be linked to PPG readings from a smartwatch or other device.

By implementing these diverse annotation methods, the system 100 ensures that its first dataset is not limited to a single method of BP measurement. Instead, it incorporates oscillometric, auscultatory, invasive, and automated readings, covering clinical, consumer, and real-world BP data sources. This broad annotation approach improves the model's adaptability, allowing it to be calibrated for various environments and user populations, ensuring higher accuracy and regulatory compliance.

As described above, the first dataset may be a heterogenous dataset including BP values collected from the first cohort using various sources. In some examples, the second dataset is a homogenous dataset collected from the second cohort using a single (or) reference source. In some examples, the reference source used for the second dataset corresponds to a reference methodology used in a clinical setting (e.g., a clinical trial). In some examples, the reference source used for the second dataset corresponds to one or more of the collection/annotation methods described above with respect to first dataset.

In some examples, the system 100 (e.g., the model engine 126) is configured to ensure each signal (or measurement) meets a predetermined quality standard (or criteria) prior to being added to the first or second datasets. The quality standard may be applied to prevent the introduction of noise, errors, or inconsistencies into the dataset(s). In some examples, the predetermined quality standard corresponds to one or more quality thresholds that are applied to the signals (or measurements). Below are multiple examples demonstrating how quality validation can be implemented using various signal processing and information processing techniques. It should be appreciated that the following examples are not intended to be limiting and may be used as standalone techniques or techniques that are combined to produce a complete standard.

Technique 1: Signal-to-Noise Ratio (SNR) Analysis. The system 100 may compute the SNR by comparing the power of a pulsatility signal to the background noise level. If the SNR falls below a predefined threshold, the signal may be rejected or flagged for further processing. This method ensures that captured signals are sufficiently strong relative to external noise sources, such as ambient light fluctuations (e.g., in PPG sensors) or electromagnetic interference (e.g., in radar-based sensors).

Technique 2: Motion Artifact Detection and Compensation. Wearable devices are often subject to movement, which can introduce distortions in the signal. The system can use accelerometer and gyroscope data to detect excessive motion and apply real-time correction algorithms, such as adaptive filtering or wavelet-based artifact removal. If motion levels exceed a threshold where correction is ineffective, the system can discard the affected signal segment and prompt the user to remain still.

Technique 3: Morphological Analysis of the Pulsatility Waveform. The system 100 may analyze the shape of a captured pulsatility waveform to determine whether it conforms to expected physiological patterns. This can include assessing peak-to-peak intervals, dicrotic notches, or the smoothness of the waveform. If the waveform exhibits excessive irregularities, such as missing peaks or flattened segments, it may be rejected as low quality.

Technique 4: Heart Rate Consistency Check. An estimated heart rate derived from the pulsatility signal may be cross-checked against a secondary heart rate measurement (e.g., from an ECG sensor or an accelerometer-based heart rate monitor). If the heart rate estimation from the pulsatility signal deviates significantly from the expected range, the signal may be flagged as unreliable.

Technique 5: Frequency Domain Analysis. The system 100 may perform a Fourier transform or wavelet transform on a pulsatility signal to examine its spectral components. In some examples, a valid signal is expected to exhibit frequency characteristics within the expected physiological range for heart rate (e.g., 0.5 Hz to 3.0 Hz). If excessive power is detected outside this range, it may indicate the presence of noise, baseline drift, or unrelated physiological signals, prompting rejection of the signal.

Technique 6: Pulse Amplitude Variability Assessment. The system 100 may evaluate the variability of pulse amplitudes over a short period. While some degree of natural variation is expected, excessive fluctuation may indicate sensor misplacement, poor contact, or environmental interferences. If the coefficient of variation in pulse amplitude exceeds a predefined threshold, the signal may be classified as low quality.

Technique 7: Baseline Wander Removal and DC Component Analysis. Pulsatility signals often exhibit slow-varying drifts due to movement, respiration, or temperature fluctuations. The system 100 may apply a high-pass filter or adaptive baseline correction algorithm to remove unwanted baseline wander. If the magnitude of the removed baseline component exceeds an acceptable threshold, it suggests poor-quality data, leading to signal rejection.

Technique 8: Multi-Sensor Data Fusion for Signal Validation. If a measurement device includes multiple sensors (e.g., combining PPG and ECG), the system 100 may perform cross-validation between different data streams. For instance, the pulse transit time (PTT) measured between an ECG peak and a PPG peak should be physiologically plausible. If inconsistencies are detected between multiple sensor modalities, the system 100 may identify unreliable segments and exclude them from analysis.

Technique 9: Adaptive Machine Learning-Based Signal Classification. In some examples, the system 100 includes a ML model, such as a convolutional neural network (CNN) or a recurrent neural network (RNN), that is trained on labeled datasets containing both high-quality and low-quality pulsatility signals. The system 100 classifies incoming signals in real time, rejecting those predicted as unreliable based on learned features such as waveform regularity, amplitude stability, and frequency content. This approach allows for adaptive improvement over time as more real-world data is collected.

Technique 10: Dynamic User Feedback for Signal Optimization. To ensure high-quality signal acquisition, the system 100 may incorporate a real-time user feedback mechanism. If the signal does not meet the quality threshold, the system 100 can prompt the user to adjust sensor placement, reduce movement, or improve skin contact. This can be achieved via haptic feedback (e.g., vibrations), visual indicators (e.g., LED signals), or on-screen instructions, ensuring that only properly acquired signals contribute to the dataset.

These examples provide diverse techniques for validating signal quality in real time, ensuring that only high-integrity pulsatility signals are processed for BP estimation. By systematically filtering out poor-quality data, the system maintains the accuracy of individual readings and preserves the integrity of large-scale datasets used for model refinement.

In addition to evaluating signal quality (or integrity), the system 100 may ensure the reliability of acquired signals (and measurements) by assessing physiological conditions and external factors at the time of recording/collection. In some examples, this multi-dimensional validation approach enhances the robustness of the system 100 by contextualizing each BP estimation with additional metadata, ensuring that only clinically meaningful and relevant signals contribute to the dataset(s).

Physiological conditions can affect BP and vascular dynamics, making it useful to contextualize a given pulsatility signal within the user's physiological state. Below are multiple examples demonstrating how contextual validation can be implemented using physiological information. It should be appreciated that the following examples are not intended to be limiting and may be used as standalone techniques or techniques that are combined to produce a complete standard.

Example 1: Day/Night Cycle Detection (Circadian Rhythm Assessment). BP follows a circadian rhythm, with lower values during sleep and higher values in wakefulness. In some examples, the system 100 leverages real-time clock data from the wearable or smartphone to classify the recording as daytime or nighttime. Wearable-based light sensors and ambient light exposure can further refine this classification.

Example 2: Physical Activity Level Assessment via Accelerometer & Gyroscope Data. Movement-based classification can be performed using accelerometer and gyroscope data to determine if the subject is at rest, walking, running, or engaged in high-intensity activity. If excessive movement is detected, the system 100 may either discard the PPG recording or adjust its interpretation.

Example 3: Heart Rate Variability (HRV) for Stress and Relaxation Detection.

HRV analysis provides insights into autonomic nervous system balance (e.g., sympathetic vs. parasympathetic dominance). In some examples, the system 100 extracts HRV metrics from PPG or ECG signals to determine whether the subject is relaxed or stressed. PPG-derived HRV may be validated against respiratory rate and electrodermal activity.

Example 4: Blood Pressure Medication and Treatment Status. Many users monitor BP while undergoing medical treatment. In some examples, the system 100 is configured to integrate with third-party systems (e.g., Apple HealthKit, Google Fit, or medical apps) that allow the system 100 to fetch medication logs and treatment history. For example, if a subject has recently taken antihypertensive drugs, the system 100 can tag the recording accordingly.

Example 5: Respiratory Rate Monitoring via PPG or Additional Sensors. Respiration influences cardiovascular parameters and pulse wave dynamics. In some examples, the system 100 extracts respiratory rate either from PPG waveform oscillations or dedicated respiration sensors in wearables (e.g., bioimpedance sensors). Abnormal respiratory rates (e.g., tachypnea, bradypnea) may indicate stress, illness, or poor measurement conditions.

Example 6: Postural State Assessment (Lying, Sitting, Standing). BP measurements may be dependent on posture. In some examples, the system 100 determines user posture using an embedded accelerometer and gyroscope to classify the position (e.g., as supine, sitting, or standing). If the subject has recently transitioned from a lying to standing position, orthostatic effects may require adjusting signal interpretation.

Example 7: Sleep State Analysis Using Wearable Sleep Tracking Sensors. If a measurement device supports sleep tracking, the system 100 may determine if the subject was asleep or awake at the time of measurement. Wearables with actigraphy-based sleep detection (e.g., motion+HRV) may help tag signals recorded in deep sleep, REM sleep, or wakefulness.

Example 8: Electrodermal Activity (EDA) for Emotional State Classification. Some wearables include electrodermal activity sensors to detect changes in skin conductance caused by emotional stress or excitement. If an EDA spike is detected, the system 100 may infer that the subject is in an emotionally heightened state and label the recording accordingly.

Example 9: Core and Peripheral Body Temperature Measurement. Certain wearables incorporate temperature sensors to monitor thermoregulation, which may influence BP variability. The system 100 may use elevated core temperature (e.g., fever, exercise) to adjust the validity of the BP estimation.

Example 10: Integration with Smartphone Sensors and Digital Biomarkers. In some examples, the system 100 can fetch additional contextual data from the user's smartphone, such as screen usage, notification activity, or app interactions, to infer stress or alertness levels. For example, high smartphone interaction during a BP recording might suggest heightened cognitive activity, impacting vascular tone.

External environmental conditions can affect BP variability and vascular function, making it useful to incorporate external contextual information into signal validation. Below are multiple examples demonstrating how contextual validation can be implemented using environmental information. It should be appreciated that the following examples are not intended to be limiting and may be used as standalone techniques or techniques that are combined to produce a complete standard.

Example 1: Temperature and Humidity Monitoring. Some measurement devices have built-in temperature and humidity sensors, or can retrieve weather data from GPS-based weather APIs. If the ambient temperature is extremely cold or hot, it may influence peripheral vasoconstriction or vasodilation, altering pulse wave characteristics.

Example 2: Atmospheric Pressure and Altitude Data from GPS and Barometers. Some measurement devices are equipped with barometric pressure sensors that can track altitude changes, such as ascending to high elevations where oxygen levels and BP behavior shift. The system 100 may tag recordings made at extreme altitudes for proper contextualization.

Example 3: Geolocation-Based Environmental Factors (Urban vs. Rural Setting). GPS integration can provide insights into whether the subject is in a high-stress urban setting or a quiet, rural environment. Prolonged exposure to noise pollution in cities may correlate with increased cardiovascular stress.

Example 4: Weather Conditions (Rain, Snow, Air Pollution Index). BP measurements can be affected by air pollution, which alters endothelial function and vascular reactivity. In some examples, the system 100 fetches real-time Air Quality Index (AQI) values and classifies the PPG signal accordingly.

Example 5: Time of the Year (Seasonal Variability). Seasonal variations in BP are well-documented, with higher readings in winter and lower in summer. In some examples, the system 100 integrates calendar-based seasonal tags into the signal validation process.

Example 6: Day of the Week (Workday vs. Weekend Influence). BP patterns may differ between weekdays and weekends, often due to work-related stress. In some examples, the system 100 tags PPG recordings based on whether they occur on a workday or a weekend.

Example 7: Circadian Misalignment (Jet Lag, Shift Work Detection). If GPS data suggests recent long-distance travel across time zones, the system 100 may infer potential circadian misalignment, which may impact vascular tone. Similar detection may be applied for night shift workers, where their biological rhythm differs from the general population.

Example 8: Lunar Cycle and Atmospheric Magnetism (Experimental Biomarkers). In some cases, there may be a correlation between lunar phases and cardiovascular rhythms. In some examples, the system 100 integrates moon phase metadata to investigate long-term trends in arterial pulsatility variability.

Example 9: Electromagnetic Field Exposure (Urban Radiation and RF Interference). Devices exposed to high electromagnetic radiation environments (e.g., industrial zones, hospitals with MRI machines) may experience PPG signal interference due to induced noise in photodiodes and analog electronics. The system 100 may detect nearby high-RF zones using a built-in magnetometer or smartphone-based RF scanners.

Example 10: Integration with Smart Home Devices for Environmental Context. If the user's smart home system includes environmental sensors (e.g., smart thermostats, humidity monitors, smart lighting), the system 100 may retrieve additional room conditions (e.g., temperature, CO2 levels) to further qualify the PPG signal.

By integrating physiological conditions and external factors into the validation process, the system 100 ensures that each acquired pulsatility signal is contextualized within a rich metadata framework. This multi-layered validation prevents misleading data from polluting the dataset(s) and allows for more accurate, reliable, and clinically meaningful BP estimations

In some examples, the characteristics of the first cohort associated with the first dataset can be selected to match the distribution of key demographic, physiological, and personal characteristic features of the second cohort associated with the second dataset. This may be advantageous when the second cohort is pre-defined by regulatory requirements or specific clinical trial inclusion criteria. By ensuring alignment, the model is trained on a dataset that adequately represents the population on which it will be validated, reducing bias and increasing generalizability.

The characterization of the first and second cohorts can include, but is not limited to, the following potential subject features: Systolic Blood Pressure (SBP) distribution, Diastolic Blood Pressure (DBP) distribution, Pulse Pressure (PP), Mean Arterial Pressure (MAP), Heart Rate Variability (HRV), age range and distribution, biological sex distribution, ethnicity and genetic ancestry, skin tone (to account for optical sensor variability in PPG recordings), Body Mass Index (BMI) distribution, height range and distribution, weight range and distribution, waist-to-hip ratio, body fat percentage, resting metabolic rate, peripheral vs. central adiposity, handedness (i.e., left vs. right dominance), vascular elasticity and arterial stiffness metrics, lipid profile (e.g., cholesterol levels, triglycerides, LDL/HDL ratios), diabetes prevalence (e.g., type 1, type 2, prediabetes status), physical activity levels (e.g., sedentary, moderate, high activity users), sleep pattern classification (e.g., insomnia, normal, sleep apnea patients), smoking history (e.g., never, current, former smokers), alcohol consumption habits, dietary habits (e.g., high sodium, low-carb, vegetarian, etc.), socioeconomic status (e.g., income, education level, access to healthcare), environmental factors (e.g., air pollution exposure, temperature, humidity), caffeine consumption levels, medication use (e.g., antihypertensive drugs, vasodilators, stimulants, etc.), and stress level classifications (e.g., based on HRV, electrodermal activity, or self-reported measures).

In some examples, the first dataset is curated and sampled to statistically match the characteristics of the second dataset. The rationale is that if the training cohort mirrors the test/validation cohort, the trained BP estimation model will perform optimally in the regulatory-defined evaluation dataset. This may relevant when the second dataset is established by regulatory authorities, imposing strict demographic and physiological distribution requirements, the target deployment population is already known, and ensuring matching distributions minimizes performance deviations in real-world settings, and/or the sensor and acquisition conditions are expected to be similar, ensuring a seamless transition from model training to deployment. To achieve this alignment between the first and second datasets, the system 100 may pre-screen participants to ensure the first dataset aligns with predefined statistical distributions, adjust sampling weights during data collection to avoid over-representation of any subgroup, apply stratified sampling techniques to ensure subgroups are proportionally represented, and/or limit recruitment of over-represented populations and actively seek underrepresented ones.

In some examples, the statistical representation of certain subgroups is modified to influence model convergence. This may be relevant when previous versions of the model exhibited performance biases in specific populations (e.g., underestimating BP in low-BMI individuals, overestimating BP in users with darker skin tones due to PPG absorption effects), the model needs to learn to generalize across underrepresented populations, even if the validation dataset is not fully balanced, and/or regulatory compliance mandates minimum performance thresholds across multiple subgroups, necessitating targeted training on weaker-performing categories. In some examples, the system 100 is configured to oversample underrepresented subgroups to ensure the model learns from their features, downsample overrepresented subgroups to avoid biases that lead to overfitting, and/or introduce synthetic data augmentation for groups where real-world data is limited.

When regulatory bodies define strict demographic quotas for validation datasets, the first dataset may be adapted to meet such standards. This can include ensuring compliance with ISO, FDA, and CE requirements for BP measurement validation, matching regulatory-mandated ratios of ethnic diversity, BMI distribution, and BP classifications (e.g., requiring a minimum percentage of hypertensive subjects for model validation), and/or proactively adjusting the first dataset to ensure it meets or exceeds generalizability and fairness criteria imposed by governing bodies.

Beyond initial dataset collection, the system 100 may implement adaptive sampling strategies for continuous model updates. For example, the system 100 may use dynamic cohort recruitment based on real-world model performance (e.g., identifying weak performance in elderly users and recruiting more data from that subgroup), feedback-driven data collection, where real-world deployment data informs gaps in model accuracy, prompting targeted cohort expansion, and/or iterative model refinements, where dataset distributions are periodically adjusted to ensure performance consistency across evolving regulatory or population demographics.

By implementing cohort engineering strategies, the system 100 ensures that its first dataset is statistically optimized for real-world performance while complying with regulatory constraints. Whether through direct matching, intelligent over/under-sampling, or regulatory-driven structuring, this approach guarantees optimal model generalizability, fairness, and clinical accuracy when evaluated in controlled validation settings.

The method 1200 provides a scalable approach to training AI/ML models for BP estimation, while maintaining high standards of accuracy required for regulatory approval. By combining large-scale, weakly labeled data collection with fine-tuning on small, high-quality datasets, the method 1200 produces a trained BP estimation model tailored for non-invasive BP monitoring. The structured training methodology ensures that the final model achieves both scalability and clinical-grade accuracy, enabling the widespread deployment of calibration-free BP estimation in consumer health devices, medical wearables, and telemedicine applications. The training methodology not only represents a fundamental innovation in the training of physiological signal models but also provides a blueprint for the development of future medical AI systems that require both large-scale adaptability and strict regulatory compliance.

While the above embodiments described the BP estimation model as a “calibration-free” model, it should be appreciated that the BP estimation model may be used with various calibration methodologies. In some examples, different calibration methodologies influence how the model processes pulsatility signals and returns BP values. For example, the BP estimation model may operate without calibration, or using different calibration techniques, such as calibrations using physiological data or direct BP data from the user 110. In some examples, the BP estimation model operates using different normalization techniques, such as normalization using morphological data. The choice of calibration or normalization strategy may impact accuracy, usability, regulatory compliance, and scalability.

Below are multiple examples of calibration strategies that may be implemented using the BP estimation model. It should be appreciated that the following examples are not intended to be limiting and may be used as standalone strategies or partial strategies that are combined to produce a complete strategies.

Strategy 1: Non-Calibrated System—Direct BP Estimation Without Any Further Calibration. Under this strategy, the system 100 functions as a fully calibration-free BP monitor, meaning that users do not need to provide any additional input beyond wearing the device or positioning the sensor correctly. The BP estimation model estimates BP using only the pulsatility signal, leveraging a large-scale, pre-trained dataset that accounts for inter-individual variability. This strategy may be advantageous because (i) there is no user burden, allowing for seamless operation without requiring calibration, (ii) it is highly scalable, as users do not need additional equipment or manual intervention, and (iii) it is ideal for consumer electronics, including smartphone-based and wearable-based BP monitoring solutions. One example implementation is a smartphone-based BP estimation, where users place their fingertip on the camera, and the system predicts BP without requiring calibration. Another example implementation is a fully automated PPG-based smartwatch or ring that continuously monitor BP without requiring external cuff measurements or user input.

Strategy 2: Normalization Using Personal Characteristics of the User. Under this strategy, the BP estimation model returns the BP value as the output following normalization using personal characteristics of the user 110. This strategy does not require an external BP monitor, but instead adjusts the model's outputs based on user-specific characteristics, such as wrist circumference, finger size, weight, height, age, and other biometric parameters. The personal characteristic features serve as a correction factor, allowing the BP estimation model to account for inter-user variations. This strategy may be advantageous because (i) it eliminates the need for a cuff-based calibration step, making it more user-friendly than traditional BP monitors and (ii) it allows for scalability in devices where an external BP calibration is impractical. In other words, this strategy is highly scalable due to the fact that there is no need for users to purchase, acquire, or access a cuff device for initialization/calibration. One example implementation is a next-generation wrist-worn BP monitor that does not require a BP cuff for calibration. Instead, it asks the user 110 for personal inputs (e.g., height, weight, wrist size, and skin tone) during setup, refining the BP prediction model accordingly. Another example implementation is a smartphone camera-based PPG BP monitor that similarly adjusts BP estimates based on user-reported biometric data. Another example implementation is a smart ring or earbud that personalizes BP readings by considering finger circumference, ear canal size, and vascular compliance metrics extracted from optical or electrical impedance sensors.

Strategy 3: Calibration Using Physiological Data from the User. Under this strategy, the BP estimation model returns the BP value as the output following calibration using physiological data from the user 110. This strategy does not require an external BP monitor or manual user input but instead relies on physiological indicators obtained from the user's own cardiovascular system. Examples of these indicators include: Pulse Transit Time (PTT) (e.g., measured from the time difference between ECG R-wave peaks and PPG pulse arrival, Heart Rate Variability (HRV) (e.g., used to infer autonomic nervous system balance and vascular tone), and Arterial Stiffness Estimation (e.g., derived from multiple PPG wavelengths or tonometry sensors). This strategy may be advantageous because (i) it is a fully self-contained calibration without needing user intervention and (ii) it enables continuous adaptation, as physiological calibration can dynamically adjust over time. One example implementation is an ECG/PPG hybrid device, such as a smartwatch that estimates BP using PTT and automatically calibrates based on beat-to-beat measurements. Another example implementation is a wearable that uses respiratory rate, arterial compliance, or hydration levels to fine-tune BP predictions without requiring external BP input.

Strategy 4: Calibration Using Blood Pressure Data from the User. Under this strategy, the model returns the BP value as the output following calibration using BP data from the user 110. The system 100 requires the user to provide a reference BP reading from an external BP device, such as an oscillometric cuff. This reference value is used to fine-tune the BP estimation model for improved accuracy. This strategy may be advantageous because (i) it provides higher accuracy by aligning model predictions with real BP readings and (ii) it allows for dynamic recalibration, enabling users to update the system 100 if vascular properties change over time. One example implementation is a wrist-worn PPG-based BP monitor that requires an initial calibration using an integrated oscillometric cuff. The model adjusts itself based on the user's real BP values to improve accuracy. Another example implementation is a smartwatch that requires the user to manually calibrate the watch using an external BP cuff and repeat the process periodically. Another example implementation is a wearable integrating with home BP cuffs, where users input their BP readings manually, and the system updates its predictions accordingly.

Strategy 5: Hybrid and Adaptive Calibration Strategies. Under this strategy, multiple calibration or normalization strategies may be combined to balance accuracy, usability, and scalability. Examples of hybrid calibration approaches include: periodic cuff calibration with personal characteristic normalization adjustment, a device that initially calibrates with a BP cuff, but then applies personal characteristic data-based corrections to normalize and reduce the need for repeated cuff calibrations, a device that reduces the need for recurrent cuff use by incorporating personal characteristic normalization adjustments from wrist size and skin tone, physiological calibration with on-demand user input, a system that automatically calibrates using physiological signals but allows users to input occasional BP readings for fine-tuning, a smartwatch that uses PPG and ECG-derived PTT but allows the user to enter manual BP values periodically to maintain accuracy, no calibration for general users but optional calibration for advanced users, a system that functions entirely calibration-free by default but offers an optional calibration mode for users who prefer higher accuracy with manual input, and a smartphone-based BP estimator that operates without calibration but allows users to input BP manually for improved precision.

### Hardware and Software Implementations

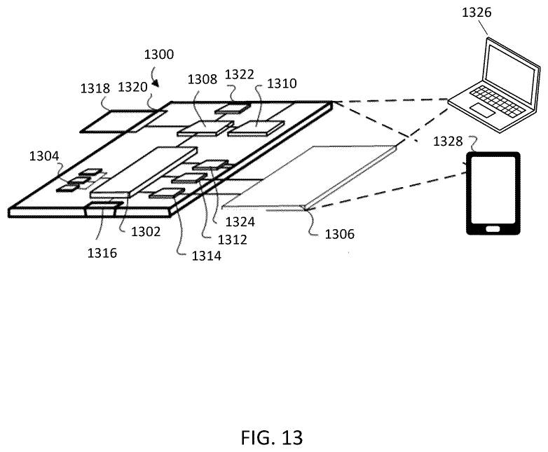

FIG. 13 shows an example of a generic computing device 1300, which may be used with some of the techniques described in this disclosure (e.g., as user device 102a, 102b or application server 124). Computing device 1300 includes a processor 1302, memory 1304, an input/output device such as a display 1306, a communication interface 1308, and a transceiver 1310, among other components. The device 1300 may also be provided with a storage device, such as a micro-drive or other device, to provide additional storage. Each of the components 1300, 1302, 1304, 1306, 1308, and 1310, are interconnected using various buses, and several of the components may be mounted on a common motherboard or in other manners as appropriate.

The processor 1302 can execute instructions within the computing device 1300, including instructions stored in the memory 1304. The processor 1302 may be implemented as a chipset of chips that include separate and multiple analog and digital processors. The processor 1302 may provide, for example, for coordination of the other components of the device 1300, such as control of user interfaces, applications run by device 1300, and wireless communication by device 1300.

Processor 1302 may communicate with a user through control interface 1312 and display interface 1314 coupled to a display 1306. The display 1306 may be, for example, a TFT LCD (Thin-Film-Transistor Liquid Crystal Display) or an OLED (Organic Light Emitting Diode) display, or other appropriate display technology. The display interface 1314 may comprise appropriate circuitry for driving the display 1306 to present graphical and other information to a user. The control interface 1312 may receive commands from a user and convert them for submission to the processor 1302. In addition, an external interface 1316 may be provided in communication with processor 1302, so as to enable near area communication of device 1300 with other devices. External interface 1316 may provide, for example, for wired communication in some implementations, or for wireless communication in other implementations, and multiple interfaces may also be used.

The memory 1304 stores information within the computing device 1300. The memory 1304 can be implemented as one or more of a computer-readable medium or media, a volatile memory unit or units, or a non-volatile memory unit or units. Expansion memory 1318 may also be provided and connected to device 1300 through expansion interface 1320, which may include, for example, a SIMM (Single In Line Memory Module) card interface. Such expansion memory 1318 may provide extra storage space for device 1300, or may also store applications or other information for device 1300. Specifically, expansion memory 1318 may include instructions to carry out or supplement the processes described above, and may include secure information also. Thus, for example, expansion memory 1318 may be provided as a security module for device 1300, and may be programmed with instructions that permit secure use of device 1300. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer-or machine-readable medium, such as the memory 1304, expansion memory 1318, memory on processor 1302, or a propagated signal that may be received, for example, over transceiver 1310 or external interface 1316.

Device 1300 may communicate wirelessly through communication interface 1308, which may include digital signal processing circuitry where necessary. Communication interface 1308 may in some cases be a cellular modem. Communication interface 1308 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1310. In addition, short-range communication may occur, such as using a Bluetooth, WiFi, or other such transceiver (not shown). In addition, GPS (Global Positioning System) receiver module 1322 may provide additional navigation-and location-related wireless data to device 1300, which may be used as appropriate by applications running on device 1300.

Device 1300 may also communicate audibly using audio codec 1324, which may receive spoken information from a user and convert it to usable digital information. Audio codec 1324 may likewise generate audible sound for a user, such as through a speaker, e.g., in a handset of device 1300. Such sound may include sound from voice telephone calls, may include recorded sound (e.g., voice messages, music files, etc.) and may also include sound generated by applications operating on device 1300. In some examples, the device 1300 includes a microphone to collect audio (e.g., speech) from a user. Likewise, the device 1300 may include an input to receive a connection from an external microphone.

The computing device 1300 may be implemented in a number of different forms, as shown in FIG. 13. For example, it may be implemented as a computer (e.g., laptop) 1326. It may also be implemented as part of a smartphone 1328, smart watch, tablet, personal digital assistant, or other similar mobile device.

Some implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing.

The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation.

Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1. A system for determining a blood pressure value of a user comprising:
a processor programmed to perform operations comprising:
receiving an indication of a type of a sensor;
receiving a signal from the sensor, the sensor adapted to non-invasively receive the signal from the user;
validating that at least one of the type of the sensor or the received signal satisfies one or more predetermined criteria;
delivering the type of the sensor and the signal as inputs to a model, the model configured to analyze the signal based on the type of the sensor and, without relying on a user-specific calibration measurement, return a blood pressure value as an output; and
delivering the output blood pressure value to the user.

2. The system of claim 1, wherein the sensor comprises a video acquisition system embedded within a smartphone, a tablet, or any handheld electronic device.

3. The system of claim 1, wherein the sensor comprises an optical sensor housed in a wearable device, a smartwatch, a wristband, or a finger-worn ring.

4. The system of claim 1, wherein the signal comprises a pulsatility signal from the arteries of the user.

5. The system of claim 1, wherein validating the sensor comprises verifying that the sensor generates signals that comply with the one or more predetermined criteria.

6. The system of claim 1, wherein validating the received signal comprises evaluating signal integrity based on one or more of signal-to-noise ratio, waveform stability, artifact detection, or physiological plausibility.

7. The system of claim 1, wherein validating the received signal comprises confirming compliance of the received signal with regulatory standards or guidelines applicable to the territory in which the blood pressure value is delivered.

8. The system of claim 1 further comprising:
delivering at least one personal characteristic of the user as an input to the model,
wherein the model is configured to analyze the signal and the at least one personal characteristic and, without any further calibration, return the blood pressure value based on the signal and the at least one personal characteristic.

9. The system of claim 1, wherein delivering the output blood pressure value comprises delivering the blood pressure value in a user-accessible format.

10. A system for determining a blood pressure value of a user comprising:
a processor in communication with a calculation model, wherein the calculation model is configured to, without relying on a user-specific calibration measurement, determine the blood pressure value of the user based on signals collected from the user, wherein the processor is programmed to perform operations comprising:
receiving an indication of a type of a measurement device;
identifying whether the measurement device is a verified device, verified devices defined as measurement devices that have completed a qualification process, wherein the qualification process comprises:
analyzing a shape of a signal received from the measurement device;
determining configuration settings for the measurement device such that the shape of the signal will have predetermined characteristics;
storing the configuration settings; and
qualifying the measurement device as a verified device and
if the measurement device is a verified device, further performing operations comprising:
configuring the measurement device according to configuration settings determined during the qualification process;
receiving a signal from the measurement device; and
delivering the type of the measurement device and the signal as inputs to the calculation model to determine the blood pressure value of the user based on the type of the measurement device.

11. The system of claim 10, wherein the signals comprise PPG signals.

12. The system of claim 10, wherein the measurement device comprises a smartphone or a wearable device.

13. The system of claim 10, wherein the qualification process comprises benchtop testing using at least one of simulated data and real subject data.

14. The system of claim 10, wherein analyzing the shape of the signal comprises evaluating one or more morphological, statistical, physiological, or model-derived characteristics of the signal, wherein the characteristics include at least one of amplitude metrics, temporal features, waveform slopes, baseline stability, distortion or noise metrics, physiological landmark presence, inter-feature relationships, quality indicators generated by the calculation model, or correlation measures with reference signals.

15. The system of claim 10, wherein the configuration settings comprise at least one of optical signal acquisition parameters, analog front-end processing parameters, digital signal processing parameters, data encoding parameters, or environmental adaptation parameters, including but not limited to light intensity, light exposure time, wavelength selection, illumination mode, emitter-detector spatial configuration, sampling rate, gain control, filtering characteristics, resolution, dynamic range, signal-to-noise ratio optimization, compression settings, frame rate, white balance, autofocus mode, exposure compensation, stabilization settings, ambient light correction, temperature compensation, or data transmission format, wherein such configuration settings are set to optimize signal quality, maintain measurement consistency, or ensure regulatory compliance across different device types and operating conditions.

16. The system of claim 10, wherein configuring the measurement device according to the configuration settings comprises at least one of: (i) modifying settings of the measurement device or (ii) determining that the settings of the measurement device already align with the configuration settings.

17. The system of claim 10, wherein the operations further comprise validating a quality of the signal received from the measurement device before delivering the signal to the calculation model.

18. The system of claim 17, wherein validating the quality of the signal comprises evaluating at least one of signal integrity characteristics, physiological plausibility criteria, contextual conditions of the user, environmental conditions during measurement, or compliance with predetermined regulatory criteria.

19. The system of claim 10, wherein the operations further comprise delivering the blood pressure value to the user.

20. The system of claim 10, wherein, if the measurement device is not included in the list of verified devices, further performing operations comprising:
causing the system to display a message indicating that the measurement device is not supported for interaction with the calculation model.

21. A system for determining a blood pressure value of a user comprising:
a processor programmed to perform operations comprising:
receiving an indication of a type of a sensor;
receiving a signal from the sensor, the sensor being adapted to receive the signal from the user;
delivering the type of the sensor and the signal as inputs to a model that returns, without relying on a user-specific calibration measurement, a blood pressure value as an output, the blood pressure value being determined from an analysis of the signal based on the type of the sensor, wherein the model is developed according to a process comprising:
training the model using a first data set of signals;
refining the trained model using a second data set of signals wherein the signals of the second data set are annotated with information correlated with blood pressure values collected using a reference methodology on a first cohort of subjects; and
validating the refined model to ensure conformance with a validation criteria by comparing blood pressure values returned by the refined model against blood pressure values collected on a second cohort of subjects using the reference methodology.

22. The system of claim 21, wherein the sensor is embedded within a smartphone or a wearable device.

23. The system of claim 21, wherein the operations further comprise validating a quality of the signal received from the sensor.

24. The system of claim 23, wherein validating the quality of the signal comprises evaluating at least two of signal integrity, physiological conditions and external factors.

25. The system of claim 21, wherein the first data set comprises pulsatility signals collected by first data set sensors, wherein the first data set sensors (i) are of the same type as the sensor, (ii) are of a different type as the sensor, or (iii) comprise of multiple sensor types.

26. The system of claim 21, wherein at least some of the signals in the first data set are annotated with information correlated with blood pressure values collected using at least one of (i) the reference methodology, (ii) a methodology different than the reference methodology, or (iii) multiple methodologies.

27. The system of claim 21, wherein the first data set is collected using scalable data collection methodologies that require at most a minimal level of clinical supervision.

28. The system of claim 21, wherein the signals of the second data set are annotated with information correlated with blood pressure values collected using multiple reference methodologies.

29. The system of claim 21, wherein the reference methodology comprises collecting measurements using at least one of an auscultatory blood pressure monitor, an invasive blood pressure monitor, a blood pressure monitor required for regulatory compliance, or a blood pressure monitor recognized by a regulatory body.

30. The system of claim 21, wherein the operations further comprise presenting the blood pressure value to the user.
